(19) United States
(12) Patent Application Publication
(10) Pub. No.: US 2026/0335557 A1
(43) Pub. Date: Oct. 29, 2026

(54) SYSTEM AND METHOD FOR INJECTION

(71) Applicant: CREDENCE MEDSYSTEMS, INC., Menlo Park, CA (US)

(72) Inventors: Alan E. Shluzas, San Carlos, CA (US); Everett Matthew Notaro, Santa Clara, CA (US)

(73) Assignee: CREDENCE MEDSYSTEMS, INC., Menlo Park, CA (US)

(21) Appl. No.: 19/540,044

(22) Filed: Feb. 13, 2026

Related U.S. Application Data
(60) Provisional application No. 63/758,909, filed on Feb. 14, 2025.
(60) Provisional application No. 63/868,131, filed on Aug. 21, 2025.

(51) Int. Cl. A61M 5/31

(52) U.S. Cl. CPC A61M 5/3146

(57) ABSTRACT

A system for injecting includes a syringe body having proximal and distal ends, a syringe interior, and a syringe flange at the proximal end thereof. The system also includes an injectable fluid disposed in the syringe interior. The system further includes a stopper member disposed in the syringe interior. Moreover, the system includes a plunger member having a distal end configured to be disposed adjacent to the stopper member. In addition, the system includes a finger flange removably coupled to the syringe flange. The system also includes a shroud movable relative to the finger flange. The plunger member is coupled to the shroud.

### BACKGROUND

The present application claims priority to U.S. Provisional Patent Application Ser. No. 63/758,909, filed Feb. 14, 2025 under attorney docket number CM.30049.00, and entitled “SYSTEM AND METHOD FOR INJECTION” and U.S. Provisional Patent Application Ser. No. 63/868, 131, filed Aug. 21, 2025 under attorney docket number CM. 30052.00, and entitled “SYSTEM AND METHOD FOR INJECTION”. This application includes subject matter similar to the subject matter described in the following co-owned U.S. patent applications: (1) U.S. Utility patent application Ser. No. 16/683, 126, filed on Nov. 13, 2019 under attorney docket number CM. 20022-1.00, and entitled “SYSTEM AND METHOD FOR MICRODOSE INJECTION”; (2) U.S. Utility patent application Ser. No. 14/696,342, filed Apr. 24, 2015 under attorney docket number CM.20003.00, and entitled “SYSTEM AND METHOD FOR SAFETY SYRINGE”; (3) U.S. Utility patent application Ser. No. 14/543,787, filed Nov. 17, 2014 under attorney docket number CM. 20002.00, and entitled “SYSTEM AND METHOD FOR DRUG DELIVERY WITH A SAFETY SYRINGE”; (4) U.S. Utility patent application Ser. No. 14/321,706, filed Jul. 1, 2014 under attorney docket number CM.20001.00, and entitled “SAFETY SYRINGE”; (5) U.S. Utility patent application Ser. No. 15/801,239, filed Nov. 1, 2017 under attorney docket number CM. 20011.00, and entitled “SYSTEM AND METHOD FOR SAFETY SYRINGE”; (6) U.S. Utility patent application Ser. No. 15/801,259, filed Nov. 1, 2017 under attorney docket number CM.20012.00, and entitled “SYSTEM AND METHOD FOR SAFETY SYRINGE”; (7) U.S. Utility patent application Ser. No. 15/801,304, filed Nov. 1, 2017 under attorney docket number CM. 20015.00, and entitled “SYSTEM AND METHOD FOR SAFETY SYRINGE; (8) U.S. Utility patent application Ser. No. 15/985,354, filed May 21, 2018 under attorney docket number CM.20016.00, and entitled “SYSTEM AND METHOD FOR COLLECTING INJECTION INFORMATION”; (9) U.S. Utility Patent Application Ser. No. 62/542,230, filed Aug. 7, 2017 under attorney docket number CM. 00.30013, and entitled “CARTRIDGE SAFETY INJECTION SYSTEM AND METHODS”; (10) U.S. Utility patent application Ser. No. 15/801,239, filed Nov. 1, 2017 under attorney docket number CM.20011.00, and entitled “SYSTEM AND METHOD FOR SAFETY SYRINGE”; (11) U.S. Utility patent application Ser. No. 15/801,259, filed Nov. 1, 2017 under attorney docket number CM. 20012.00, and entitled “SYSTEM AND METHOD FOR SAFETY SYRINGE”; (12) U.S. Utility patent application Ser. No. 15/801,281, filed Nov. 1, 2017 under attorney docket number CM.20013.00, and entitled “CARTRIDGE SAFETY INJECTION SYSTEM AND METHODS”; (13) U.S. Utility patent application Ser. No. 15/801,304, filed Nov. 1, 2017 under attorney docket number CM.20015.00, and entitled “SYSTEM AND METHOD FOR SAFETY SYRINGE”; (14) U.S. Utility patent application Ser. No. 16/011,453, filed Jun. 18, 2018 under attorney docket number CM.20014.00, and entitled “SYSTEM AND METHOD FOR SAFETY SYRINGE”; (15) U.S. Utility patent application Ser. No. 15/985,354, filed May 21, 2018 under attorney docket number CM.20016.00, and entitled “SYSTEM AND METHOD FOR COLLECTING INJECTION INFORMATION”; (16) U.S. Utility patent application Ser. No. 16/683,157, filed Nov. 13, 2019 under attorney docket number CM. 20022-2.00, and entitled “SYSTEM AND METHOD FOR MULTIPLE SITE INJECTION”; (17) U.S. Utility patent application Ser. No. 17/351,006, filed Jun. 17, 2021 under attorney docket number CM.20029.00, and entitled “SYSTEM AND METHOD FOR MICRODOSE INJECTION”; (18) U.S. Provisional Patent Application Ser. No. 63/767,424, filed Mar. 5, 2025 under attorney docket number CM.30050.00, and entitled “SYSTEM AND METHOD FOR INJECTION”; and (19) U.S. Provisional Patent Application Ser. No. 63/841,870, filed Jul. 10, 2025 under attorney docket number CM. 30051.00, and entitled “SYSTEM AND METHOD FOR INJECTION.” The contents of the applications identified herein are fully incorporated herein by reference as though set forth in full.

### FIELD OF THE INVENTION

The present invention relates generally to injection systems, devices, and processes for facilitating various levels of control over fluid infusion, and more particularly to systems and methods related to syringes for delivery microliter range doses of fluids in healthcare environments.

Millions of syringes 2, such as that depicted in FIG. 1A, are consumed in healthcare environments every day. A typical syringe 2 includes a tubular body 4, a plunger 6, and an injection needle 8. As shown in FIG. 1B, such a syringe 2 may be utilized not only to inject fluid into a patient, but also to withdraw or expel fluid out of or into a container such as a medicine bottle, vial, bag, or other drug containment system 10. Indeed, due to regulatory constraints in some countries such as the United States as well as sterility maintenance concerns, upon use of a medicine bottle 10 with a syringe 2 as shown in a particular patient's environment, such medicine bottle may only be utilized with a single patient and then must be disposed of—causing significant medical waste from bottle and remaining medicine disposal, and even contributing to periodic shortages of certain critical drugs.

Referring to FIG. 2A, three Luer-type syringes 12 are depicted, each having a Luer fitting geometry 14 disposed distally, so that they may be coupled with other devices having similar mating geometry, such as the Luer manifold assembly 16 depicted in FIG. 2B. The Luer manifold assembly of FIG. 2B may be used to administer liquid drugs to the patient intravenously with or without the use of an intravenous infusion bag. The Luer fittings 14 of the syringes of FIG. 2A may be termed the “male” Luer fittings, while those of FIG. 2B 18 may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting 14 which are configured to engage a flange on the female fitting 18 and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during the loading to provide a Luer coupling. The use of needle injection configurations, on the other hand, carries with it the risk of a sharp needle contacting or poking a person or structure that is not desired. For this reason, so called “safety syringes” have been developed.

One embodiment of a safety syringe 20 is shown in FIG. 3, wherein a tubular shield member 22 is spring biased to cover the needle 8 when released from a locked position relative to the syringe body 4. Another embodiment of a safety syringe 24 is shown in FIGS. 4A-4B. With such a configuration, after full insertion of the plunger 6 relative to the syringe body 4, the retractable needle 26 is configured to retract 28, 26 back to a safe position within the tubular body 4, as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for pre-filled syringe assemblies such as those depicted in FIGS. 5A and 5B, which generally include a syringe body, or “drug enclosure containment delivery system”, 34, a plunger tip, plug, or stopper 36, and a distal seal or cap 35 which may be fitted over a Luer type interface (FIG. 5A shows the cap 35 in place; FIG. 5B has the cap removed to illustrate the Luer interface 14. Liquid medicine may reside in the volume, or medicine reservoir, 40 between the distal seal 35 and the distal end 37 of the stopper member 36. The stopper member 36 may include a standard butyl rubber material and may be coated, such as with a biocompatible lubricious coating (e.g., polytetrafluoroethylene (“PTFE”)), to facilitate preferred sealing and relative motion characteristics against the associated syringe body 34 structure and material. The proximal end of the syringe body 34 in FIG. 5B includes a conventional integral syringe flange 38), which is formed integral to the material of the syringe body 34. The flange 38 is configured to extend radially from the syringe body 34 and may be configured to be a full circumference, or a partial circumference around the syringe body 34. A partial flange is known as a “clipped flange” while the other is known as a “full flange.” The flange is used to grasp the syringe with the fingers to provide support for pushing on the plunger to give the injection. The syringe body 34 preferably includes a translucent material such as a glass or polymer. To form a contained volume within the medicine chamber or reservoir 40, and to assist with expulsion of the associated fluid through the needle, a stopper member 36 may be positioned within the syringe body 34. The syringe body may define a substantially cylindrical shape (i.e., so that a plunger tip 36 having a circular cross sectional shape may establish a seal against the syringe body), or be configured to have other cross sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus certain suppliers have moved to more “vertical” solutions, such as that 41 featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Moreover, injection systems have reduced accuracy and precision as the volume of injectable fluid is reduced into the microliter range. In particular, removing air from the syringe body (“de-bubbling”) before injection is difficult to perform accurately and precisely for such injection systems.

There is a need for injection systems which address shortcomings of currently-available configurations. In particular, there is a need for injection systems that perform (e.g., de-bubble and inject) accurately in the microliter range. It is also desirable that such syringe assemblies may utilize the existing and relatively well-controlled supply chain of conventionally delivered pre-filled syringe bodies and other off-the-shelf components, and the corresponding assembly machinery and personnel.

### SUMMARY

Embodiments are directed to injection systems. In particular, the embodiments are directed to microliter range injection systems that include at least some off-the-shelf syringe components.

In one embodiment, a system for injecting includes a syringe body having proximal and distal ends, a syringe interior, and a syringe flange at the proximal end thereof. The system also includes an injectable fluid disposed in the syringe interior. The system further includes a stopper member disposed in the syringe interior. Moreover, the system includes a plunger member having a distal end configured to be disposed adjacent to the stopper member. In addition, the system includes a finger flange removably coupled to the syringe flange. The system also includes a shroud movable relative to the finger flange. The plunger member is coupled to the shroud.

In one or more embodiments, the plunger member is rotatably and translationally coupled to the shroud. The shroud may be rotatable relative to the finger flange. The distal end of the plunger member is coupled to the stopper member.

In one or more embodiments, the finger flange includes a proximally extending member having an external thread defined thereon. The shroud may define an inwardly extending member disposed in the external thread and configured to travel along the external thread. The proximally extending member may define a distally extending external longitudinal slot at a distal end of the external thread. The proximally extending member may define a bump at the distal end of the external thread. The bump may be configured to cooperate with the inwardly extending member to generate a tactile and/or audible indicator. The proximally extending member may define a bump adjacent a proximal end of the external thread. The bump may be configured to cooperate with the inwardly extending member to generate a tactile and/or audible indicator. The proximally extending member may define a distally extending internal longitudinal slot. The plunger member may define an elongate member outwardly extending from an exterior surface thereof. The elongate member of the plunger member may be configured to travel along the distally extending internal longitudinal slot of the proximally extending member.

In one or more embodiments, the proximally extending member defines a deflectable detent adjacent a proximal end of the external thread. The deflectable detent is configured to cooperate with the inwardly extending member to control movement of the inwardly extending member between the external thread and the distally extending external longitudinal slot. The deflectable detent may be configured to deflect radially and/or longitudinally/axially. The deflectable detent may be configured to deflect to allow the inwardly extending member to move from the external thread to the distally extending external longitudinal slot. The deflectable detent may be configured to elastically deform with application of a deflecting force to allow the inwardly extending member to move from the external thread to the distally extending external longitudinal slot, and return to an original position after the deflecting force is removed therefrom. The deflectable detent may be biased to return to an original/neutral position after a deflecting force is removed therefrom. The deflectable detent returning to the original/neutral position may prevent the inwardly extending member from moving from the distally extending external longitudinal slot to the external thread. The deflectable detent may be self-energizing to return to an original/neutral position after a deflecting force is removed therefrom. The deflectable detent may be configured to cooperate with the inwardly extending member to generate a tactile and/or audible indicator.

In one or more embodiments, the finger flange defines a flange proximally facing surface. The finger flange may include a flange elongate member proximally extending from the flange proximally facing surface. The flange elongate member may be disposed partially along an outer surface of the proximally extending member of the finger flange. The flange elongate member may define a flange elongate member proximally facing surface. The shroud may define a shroud distally facing surface configured to interfere with the flange elongate member proximally facing surface to limit distal movement of the shroud to a first longitudinal position on the finger flange when the shroud is in a first rotational state relative to the finger flange. The shroud may define a slot extending proximally from the distally facing surface and configured to receive the flange elongate member to allow distal movement of the shroud distally beyond the first longitudinal position on the finger flange when the shroud is in a second rotational state relative to the finger flange. The shroud distally facing surface may be configured to interfere with the flange proximally facing surface to limit distal movement of the shroud to a second longitudinal position on the finger flange when the shroud is in the second rotational state relative to the finger flange. The second longitudinal position may be distal of the first longitudinal position.

In one or more embodiments, the system has a transport configuration in which the plunger member is in a proximal most position, a primed/injection ready configuration in which the plunger member is in an intermediate position, and an injected configuration in which the plunger member is in a distal most position. The slot defined by the shroud may be configured to align with the flange elongate member on the finger flange when the system is in the primed/injection ready configuration. The shroud may define an external indicia of rotation direction.

In one or more embodiments, the finger flange includes a first external indicia of a first system transition between the transport configuration and the primed/injection ready configuration, and a second external indicia of a second system transition between the primed/injection ready configuration and the injected configuration. The shroud may not cover the first external indicia or the second external indicia when the system is in the transport configuration. The shroud may cover the first external indicia and may not cover the second external indicia when the system is in the primed/injection ready configuration. The shroud may cover the first external indicia and the second external indicia when the system is in the injected configuration. The first external indicia may have a first color, the second external indicia may have a second color, and the first color may be different from the second color. The first external indicia may have a first pattern, the second external indicia may have a second pattern, and the first pattern may be different from the second pattern. The first external indicia may have a first texture, the second external indicia may have a second texture, and the first texture may be different from the second texture.

In one or more embodiments, the finger flange defines a flange proximally facing surface, and the shroud defines a shroud distally facing surface configured to interfere with the flange proximally facing surface to limit distal movement of the shroud relative to the finger flange.

In one or more embodiments, plunger member includes a pair of radially outwardly extending members adjacent the thumbpad. The plunger member may define a pair of grooves adjacent a thumbpad at a proximal end thereof. The shroud may define an opening at a proximal end thereof and a pair of latches adjacent the opening configured to cooperate with the pair of grooves to couple to the plunger member to the shroud.

In another embodiment, a method for injecting a fluid includes providing a syringe assembly. The syringe assembly includes a syringe body having proximal and distal ends, a syringe interior, and a syringe flange at the proximal end thereof. The system also includes an injectable fluid disposed in the syringe interior. The system further includes a stopper member disposed in the syringe interior. Moreover, the system includes a plunger member having a thumbpad at a proximal end thereof, and a distal end configured to be disposed adjacent to the stopper member. In addition, the system includes a finger flange removably coupled to the syringe flange. The system also includes a shroud movable relative to the finger flange. The plunger member is coupled to the shroud. The method also includes rotating the shroud relative to the finger flange to the syringe body to a rotation end location to advance the shroud, the plunger member, and the stopper member relative to the finger flange. The method further includes applying a distally directed force to a proximal end of the shroud advance the shroud to a distal end location expel a portion of the injectable fluid from the syringe interior through the distal end of the syringe body.

In one or more embodiments, the syringe assembly has a transport configuration in which the plunger member is in a proximal most position, a primed/injection ready configuration in which the plunger member is in an intermediate position, and an injected configuration in which the plunger member is in the distal end position. Rotating the shroud relative to the finger flange to the rotation end location may transform the syringe assembly from the transport configuration to the primed/injection ready configuration. Applying the distally directed force to the thumbpad to advance the plunger member to the distal end position may transform the syringe assembly from the primed/injection ready configuration to the injected configuration.

In one or more embodiments, wherein the finger flange comprises a proximally extending member having an external thread defined thereon. The finger flange may define a flange proximally facing surface. The finger flange may include a flange elongate member proximally extending from the flange proximally facing surface. The flange elongate member may be disposed partially along an outer surface of the proximally extending member of the finger flange. The flange elongate member may define a flange elongate member proximally facing surface. The shroud may define a shroud distally facing surface configured to interfere with the flange elongate member proximally facing surface to limit distal movement of the shroud to a first longitudinal position on the finger flange when the shroud is in a first rotational state relative to the finger flange. The shroud may define a slot extending proximally from the distally facing surface and configured to receive the flange elongate member to allow distal movement of the shroud distally beyond the first longitudinal position on the finger flange when the shroud is in a second rotational state relative to the finger flange. The shroud distally facing surface may be configured to interfere with the flange proximally facing surface to limit distal movement of the shroud to a second longitudinal position on the finger flange when the shroud is in the second rotational state relative to the finger flange. The second longitudinal position may be distal of the first longitudinal position. Transforming the system to the primed/injection ready configuration may align the slot on the shroud with the flange elongate member on the finger flange. Transforming the system to the injected configuration may move the shroud distally facing surface into contact with the flange proximally facing surface.

In one or more embodiments, the finger flange defines a flange proximally facing surface,. The shroud may define a shroud distally facing surface,. Transforming the system to the injected configuration may move the shroud distally facing surface into contact with the flange proximally facing surface to interfere with the flange proximally facing surface to limit distal movement of the shroud relative to the finger flange.

In one or more embodiments, the finger flange includes a first external indicia of a first system transition between the transport configuration and the primed/injection ready configuration, and a second external indicia of a second system transition between the primed/injection ready configuration and the injected configuration. The shroud may not cover the first external indicia or the second external indicia when the system is in the transport configuration. Transforming the system from the transport configuration to the primed/injection ready configuration may move the shroud distally relative to the finger flange such that the shroud covers the first external indicia and does not cover the second external indicia. Transforming the system from the primed/injection ready configuration to the injected configuration may move the shroud distally relative to the finger flange such that the shroud covers the first external indicia and the second external indicia. The first external indicia may have a first color, the second external indicia may have a second color, and the first color may be different from the second color. The first external indicia may have a first pattern, the second external indicia may have a second pattern, and the first pattern may be different from the second pattern. The first external indicia may have a first texture, the second external indicia may have a second texture, and the first texture may be different from the second texture.

In one or more embodiments, the finger flange includes a proximally extending member having an external thread defined thereon. The shroud defines an inwardly extending member disposed in the external thread and configured to travel along the external thread. Rotating the shroud relative to the finger flange moves the inwardly extending member from a proximal end of the external thread to a distal end of the external thread. The proximally extending member may define a distally extending external longitudinal slot at the distal end of the external thread. Applying the distally directed force to the thumbpad may move the inwardly extending member longitudinally from a proximal end of the external longitudinal slot into the external longitudinal slot. The proximally extending member may define a proximal bump at the proximal end of the external thread and a radial bump at the distal end of the external thread. Rotating the shroud relative to the finger flange may move the inwardly extending member first past the proximal bump then past the radial bump. Moving the inwardly extending member past each of the proximal bump and the radial bump may generate a tactile and/or audible indicator.

In still another embodiment, a system for injecting includes a syringe body having proximal and distal ends, a syringe interior, and a syringe flange at the proximal end thereof. The system also includes an injectable fluid disposed in the syringe interior. The system further includes a stopper member disposed in the syringe interior. Moreover, the system includes a plunger member having a distal end configured to be disposed adjacent to the stopper member. In addition, the system includes a finger flange removably coupled to the syringe flange. The system also includes a shroud coupled to the finger flange. The plunger member is coupled to the shroud.

In one or more embodiments, the plunger member is rotatably and translationally coupled to the shroud. The shroud may be rotatably coupled to the finger flange. The distal end of the plunger member is coupled to the stopper member.

In one or more embodiments, the finger flange includes a proximally extending member having an external thread defined thereon. The shroud may define an inwardly extending member disposed in the external thread and configured to travel along the external thread. The proximally extending member may define a distally extending external longitudinal slot at a distal end of the external thread. The proximally extending member may define a bump at the distal end of the external thread. The bump may be configured to cooperate with the inwardly extending member to generate a tactile and/or audible indicator. The proximally extending member may define a bump adjacent a proximal end of the external thread. The bump may be configured to cooperate with the inwardly extending member to generate a tactile and/or audible indicator. The proximally extending member may define an inwardly extending arcuate stop and a distally extending internal longitudinal slot. The plunger member may define an elongate member outwardly extending from an exterior surface thereof. The elongate member of the plunger member may be configured to travel along the distally extending internal longitudinal slot of the proximally extending member.

In one or more embodiments, the system has a transport configuration in which the plunger member is in a proximal most position, a primed/injection ready configuration in which the plunger member is in an intermediate position, and an injected configuration in which the plunger member is in a distal most position. The finger flange may define an external indicia of the primed/injection ready configuration, the shroud may define a pointer, and the pointer on the shroud may be configured to align with the external indicia on the finger flange when the system is in the primed/injection ready configuration. The shroud may define an external indicia of rotation direction.

In one or more embodiments, the 15. the plunger member defines a pair of grooves adjacent a thumbpad at a proximal end thereof. The shroud may define an opening at a proximal end thereof and a pair of latches adjacent the opening configured to cooperate with the pair of grooves to couple to the plunger member to the shroud.

In yet another embodiment, a method for injecting a fluid includes providing a syringe assembly. The syringe assembly includes a syringe body having proximal and distal ends, a syringe interior, and a syringe flange at the proximal end thereof. The system also includes an injectable fluid disposed in the syringe interior. The system further includes a stopper member disposed in the syringe interior. Moreover, the system includes a plunger member having a thumbpad at a proximal end thereof, and a distal end configured to be disposed adjacent to the stopper member. In addition, the system includes a finger flange removably coupled to the syringe flange. The system also includes a shroud coupled to the finger flange. The plunger member is coupled to the shroud. The method also includes rotating the shroud relative to the finger flange to the syringe body to a rotation end location to advance the shroud, the plunger member, and the stopper member relative to the finger flange. The method further includes applying a distally directed force to a proximal end of the shroud advance the shroud to a distal end location expel a portion of the injectable fluid from the syringe interior through the distal end of the syringe body.

In one or more embodiments, the syringe assembly has a transport configuration in which the plunger member is in a proximal most position, a primed/injection ready configuration in which the plunger member is in an intermediate position, and an injected configuration in which the plunger member is in the distal end position. Rotating the shroud relative to the finger flange to the rotation end location may transform the syringe assembly from the transport configuration to the primed/injection ready configuration. Applying the distally directed force to the thumbpad to advance the plunger member to the distal end position may transform the syringe assembly from the primed/injection ready configuration to the injected configuration. The finger flange may define an external indicia of the primed/injection ready configuration, the shroud may define a pointer, and transforming the system to the primed/injection ready configuration may align the pointer on the shroud with the external indicia on the finger flange.

In one or more embodiments, the finger flange includes a proximally extending member having an external thread defined thereon. The shroud defines an inwardly extending member disposed in the external thread and configured to travel along the external thread. Rotating the shroud relative to the finger flange moves the inwardly extending member from a proximal end of the external thread to a distal end of the external thread. The proximally extending member may define a distally extending external longitudinal slot at the distal end of the external thread. Applying the distally directed force to the thumbpad may move the inwardly extending member longitudinally from a proximal end of the external longitudinal slot to a distal end of the external longitudinal slot. The proximally extending member may define a proximal bump at the proximal end of the external thread and a radial bump at the distal end of the external thread. Rotating the shroud relative to the finger flange may move the inwardly extending member first past the proximal bump then past the radial bump. Moving the inwardly extending member past each of the proximal bump and the radial bump may generate a tactile and/or audible indicator.

The aforementioned and other embodiments of the invention are described in the Detailed Description which follows.

### BRIEF DESCRIPTION OF THE DRAWINGS

This patent or application file contains at least one drawing executed in color. Copies of this patent or patent application publication with color drawings will be provided by the U.S. Patent and Trademark Office upon request and payment of the necessary fee.

The foregoing and other aspects of embodiments are described in further detail with reference to the accompanying drawings, in which the same elements in different figures are referred to by common reference numerals, wherein:

FIGS. 1A-5C illustrate various aspects of conventional injection syringe configurations.

FIG. 6 is a front view depicting a system for injecting according to some embodiments.

FIG. 7 is an exploded view depicting a system for injecting according to some embodiments.

FIGS. 8, 9, and 10 are perspective, side, and longitudinal cross-sectional views depicting a finger flange for use with systems for injecting according to some embodiments.

FIGS. 11, 12, 13, and 14 are perspective, longitudinal cross-sectional, bottom, and top views depicting a shroud for use with systems for injecting according to some embodiments.

FIGS. 15, 16, and 17 are perspective, side, and longitudinal cross-sectional views depicting a plunger member for use with systems for injecting according to some embodiments.

FIGS. 18, 19, 20, and 21 are perspective views depicting a system for injecting in various configurations according to some embodiments.

FIGS. 22, 23, 24, and 25 are transverse (FIGS. 22 and 23) and longitudinal (FIGS. 24 and 25) cross-sectional views depicting a system for injecting in various configurations according to some embodiments.

FIGS. 26, 27, and 28 are detailed side views depicting a shroud and a finger flange for use with systems for injecting in various configurations according to some embodiments.

FIGS. 29A, 29B, 29C, and 29D are longitudinal cross-sectional views depicting a system for injecting in various configurations according to some embodiments.

FIG. 30 is a front view depicting a system for injecting according to some embodiments.

FIG. 31 is an exploded view depicting a system for injecting according to some embodiments.

FIGS. 32, 33, and 34 are perspective, side, and longitudinal cross-sectional views depicting a finger flange for use with systems for injecting according to some embodiments.

FIGS. 35, 36, 37, and 38 are perspective, longitudinal cross-sectional, bottom, and top views depicting a shroud for use with systems for injecting according to some embodiments.

FIGS. 39, 40, 41, and 42 are perspective views depicting a system for injecting in various configurations according to some embodiments.

FIGS. 43 and 44 are longitudinal cross-sectional views depicting a system for injecting in various configurations according to some embodiments.

FIGS. 45, 46, and 47 are detailed side views depicting a shroud and a finger flange for use with systems for injecting in various configurations according to some embodiments.

FIGS. 48A, 48B, 48C, and 48D are longitudinal cross-sectional views depicting a system for injecting in various configurations according to some embodiments.

FIGS. 49A, 49B, 49C, and 49D are front views depicting a system for injecting in various configurations according to some embodiments.

FIGS. 50A, 50B, 50C, and 50D are front views depicting a system for injecting in various configurations according to some embodiments.

FIGS. 51A, 51B, 51C, and 51D are front views depicting a system for injecting in various configurations according to some embodiments.

FIGS. 52A, 52B, 52C, and 52D are front views depicting a system for injecting in various configurations according to some embodiments.

FIG. 53 is a detailed front view depicting a system for injecting according to some embodiments.

FIG. 54 is a front view depicting a system for injecting according to some embodiments.

FIG. 55 is an exploded view depicting a system for injecting according to some embodiments.

FIGS. 56, 57, and 58 are front, perspective, and longitudinal cross-sectional views depicting a finger flange for use with systems for injecting according to some embodiments.

FIGS. 59A, 59B, and 59C are transverse cross-sectional views depicting a system for injecting in various configurations according to some embodiments.

FIGS. 60A, 60B, 60C, and 60D are longitudinal cross-sectional views depicting a system for injecting in various configurations according to some embodiments.

FIG. 61 is a front view depicting a system for injecting according to some embodiments.

FIG. 62 is an exploded view depicting a system for injecting according to some embodiments.

FIGS. 63, 64, and 65 are front, perspective, and longitudinal cross-sectional views depicting a finger flange for use with systems for injecting according to some embodiments.

FIGS. 66, 67, and 68 are front views depicting a system for injecting in various configurations according to some embodiments.

FIGS. 69A, 69B, 69C, and 69D are longitudinal cross-sectional views depicting a system for injecting in various configurations according to some embodiments.

FIGS. 70A, 70B, 70C, and 70D are side views depicting a system for injecting in various configurations according to some embodiments.

FIGS. 71A, 71B, 71C, and 71D are side views depicting a system for injecting in various configurations according to some embodiments.

FIGS. 72 and 73 are front views depicting systems for injecting according to some embodiments.

FIG. 74 is an exploded view depicting a system for injecting according to some embodiments.

FIGS. 75, 76, and 77 are perspective, side, and longitudinal cross-sectional views depicting a finger flange for use with systems for injecting according to some embodiments.

FIGS. 78, 79, 80, 81, and 82 are perspective, longitudinal cross-sectional (along two different axes), and transverse cross-sectional views depicting a shroud and a plunger member (except for FIG. 82) for use with systems for injecting according to some embodiments.

FIGS. 83, 84, 85, and 86 are perspective views depicting a shroud and a plunger member mounted on a finger flange for use with systems for injecting, such as the system 600 depicted in FIGS. 72, 73, and 74, in various configurations according to some embodiments.

FIGS. 87A and 87B are longitudinal cross-sectional views depicting a shroud and a plunger member mounted on a finger flange for use with systems for injecting in two configurations according to some embodiments.

FIGS. 88A and 88B are transverse cross-sectional views depicting a shroud and a plunger member mounted on a finger flange for use with systems for injecting in two configurations according to some embodiments.

FIGS. 89A and 89B are transverse cross-sectional views depicting a shroud and a plunger member mounted on a finger flange for use with systems for injecting in two configurations according to some embodiments.

FIGS. 90A and 90B are longitudinal cross-sectional views depicting a shroud and a plunger member mounted on a finger flange for use with systems for injecting in two configurations according to some embodiments.

FIGS. 91A and 91B are longitudinal cross-sectional views depicting a shroud and a plunger member mounted on a finger flange for use with systems for injecting in two configurations according to some embodiments.

FIG. 92 is a transverse cross-sectional view depicting a shroud and a plunger member mounted on a finger flange for use with systems for injecting according to some embodiments.

FIGS. 93A and 93B are detailed front views depicting a shroud and a finger flange for use with systems for injecting in two configurations according to some embodiments.

FIG. 94 is an exploded view depicting a system for injecting according to some embodiments.

FIGS. 95 and 96 are partially exploded views depicting a system for injecting according to some embodiments.

FIG. 97 is a longitudinal cross-sectional view depicting a shroud and a plunger member mounted on a finger flange for use with systems for injecting according to some embodiments.

FIGS. 98A and 98B are detailed perspective views depicting a plunger member for use with systems for injecting according to some embodiments.

FIGS. 99, 100, and 101 are perspective, longitudinal cross-sectional, and perspective views depicting a shroud for use with systems for injecting according to some embodiments.

FIGS. 102 and 103 are perspective and longitudinal cross-sectional views depicting a shroud and a plunger member for use with systems for injecting according to some embodiments.

FIG. 104 is a front view depicting a system for injecting according to some embodiments.

FIG. 105 is an exploded view depicting a system for injecting according to some embodiments.

FIGS. 106, 107, and 108 are front, perspective, and cutaway perspective views depicting a finger flange for use with systems for injecting according to some embodiments.

FIGS. 109, 110, 111, 112, and 113 are perspective, longitudinal cross-sectional along a first axis, longitudinal cross-sectional along a second axis, bottom, and top views depicting a shroud for use with systems for injecting according to some embodiments.

FIGS. 114A, 114B, 114C, and 114D are front views depicting a system for injecting in various configurations according to some embodiments.

FIGS. 115A, 115B, 115C, and 115D are front views depicting a system for injecting in various configurations according to some embodiments.

FIGS. 116A, 116B, 116C, and 116D are side views depicting a system for injecting in various configurations according to some embodiments.

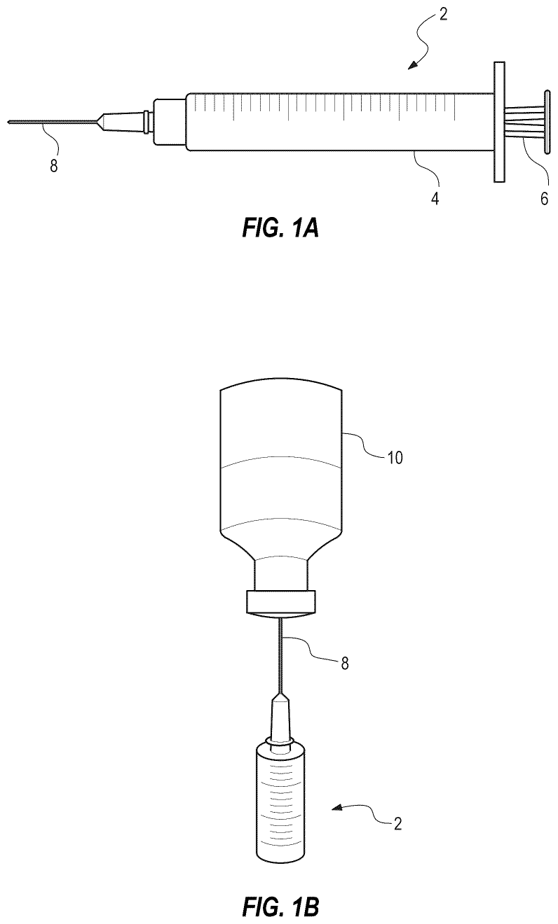

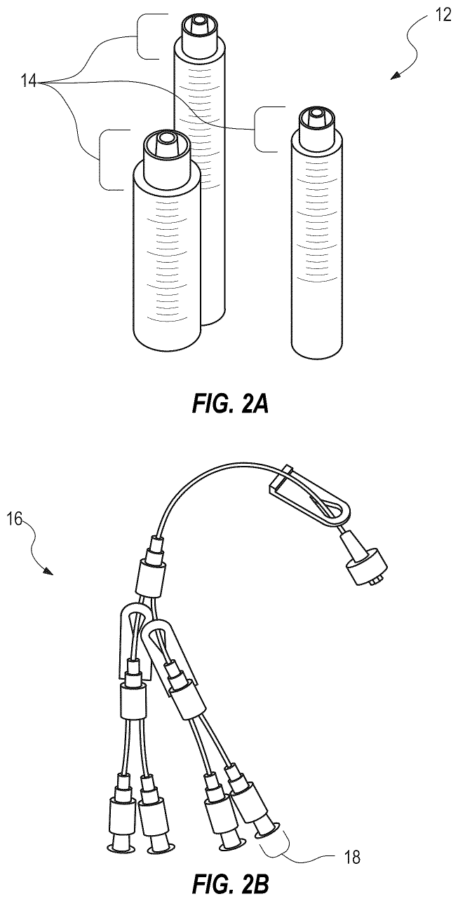

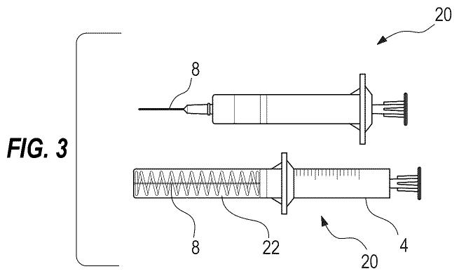

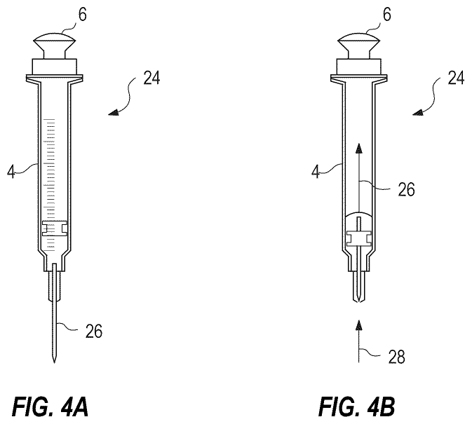

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

### Exemplary Injection Systems

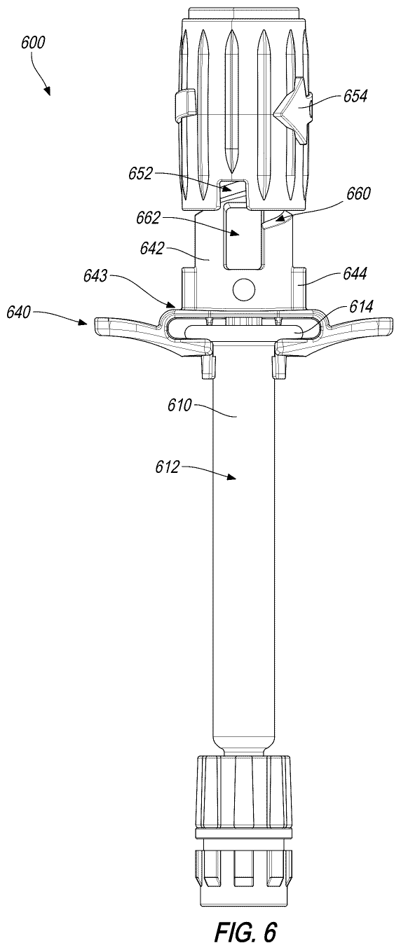

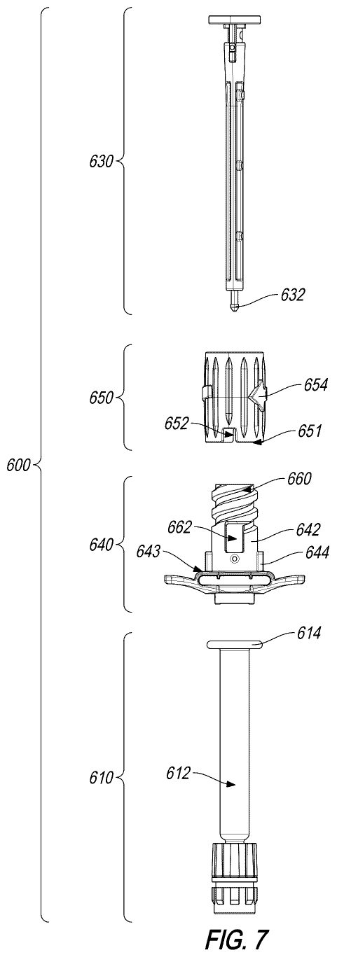

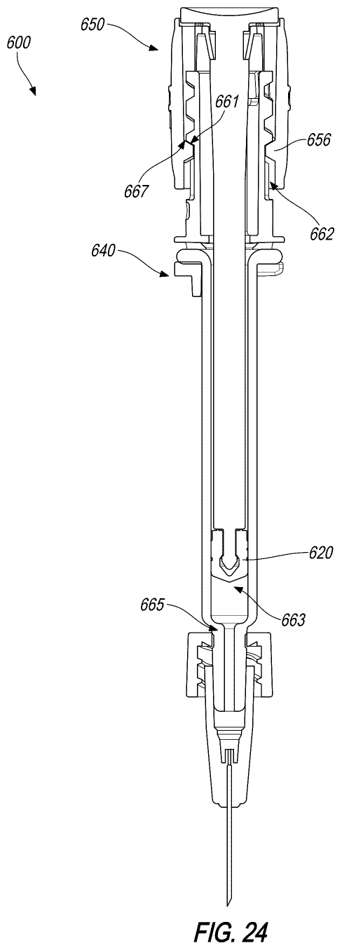

FIG. 6 is a front view depicting a system/syringe assembly 600 for injecting according to some embodiments. FIG. 7 is an exploded view depicting the system/syringe assembly 600 for injecting according to some embodiments. The system 600 includes a syringe body 610 having proximal and distal ends, a syringe interior 612, and a syringe flange 614 at the proximal end thereof. A stopper member 620 is disposed in the syringe interior 612 to form a fluid chamber therein (see FIGS. 24, 25, and 29A). The syringe interior/fluid chamber 612 contains an injectable fluid (not shown) and some air. A plunger member 630 is partially disposed in the syringe interior 612. The plunger member 630 has a distal end 632 disposed adjacent to the stopper member 620. The system 600 also includes a finger flange 640 coupled to the syringe flange 614. The system 600 further includes a shroud 650 coupled to the plunger member 630 and movable relative to the finger flange 640.

The plunger member 630 is rotatably and translationally coupled to the shroud 650. While the distal end 632 of the plunger member 630 of the system depicted in FIG. 7 has an interference type connector for a stopper member (not shown), in other embodiments the distal end of the plunger member may have a screw type connector to couple the plunger member to the stopper member or a flat end that only pushes stopper members distally without the ability to pull stopper members proximally.

The finger flange 640 defines a proximally extending member 642, which has a pair of external threads 660 defined thereon. The external threads 660 are double lead threads with about a 5 mm separation there between. The proximally extending member 642 also defines a pair of distally extending external longitudinal slot 662 at respective distal ends of the external threads 660. The finger flange 640 defines a flange proximally facing surface 643 and a pair of flange elongate members 644 proximally extending from the flange proximally facing surface 643. While a pair of flange elongate members 644 are shown, alternative embodiments may have a single flange elongate member, or 3 or more flange elongate members.

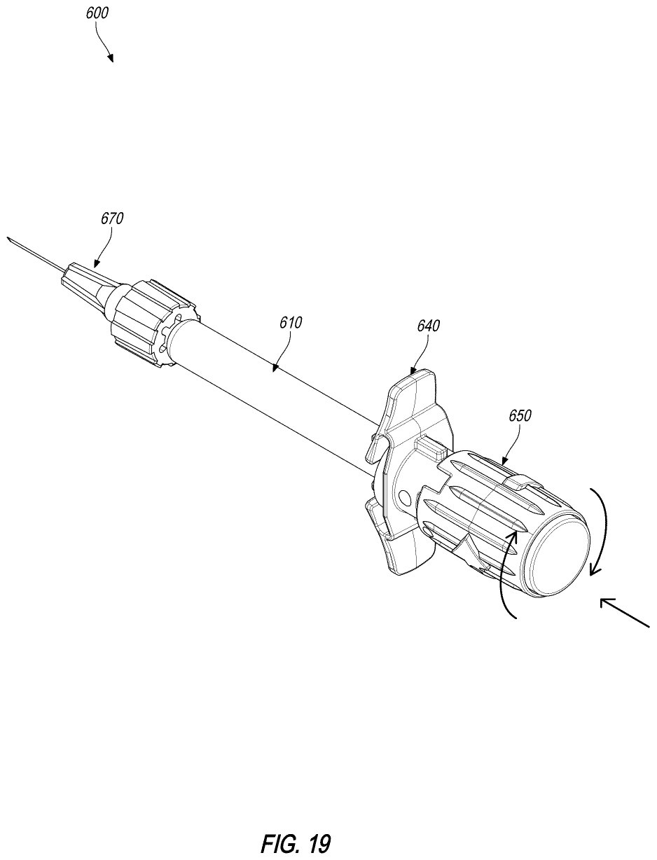

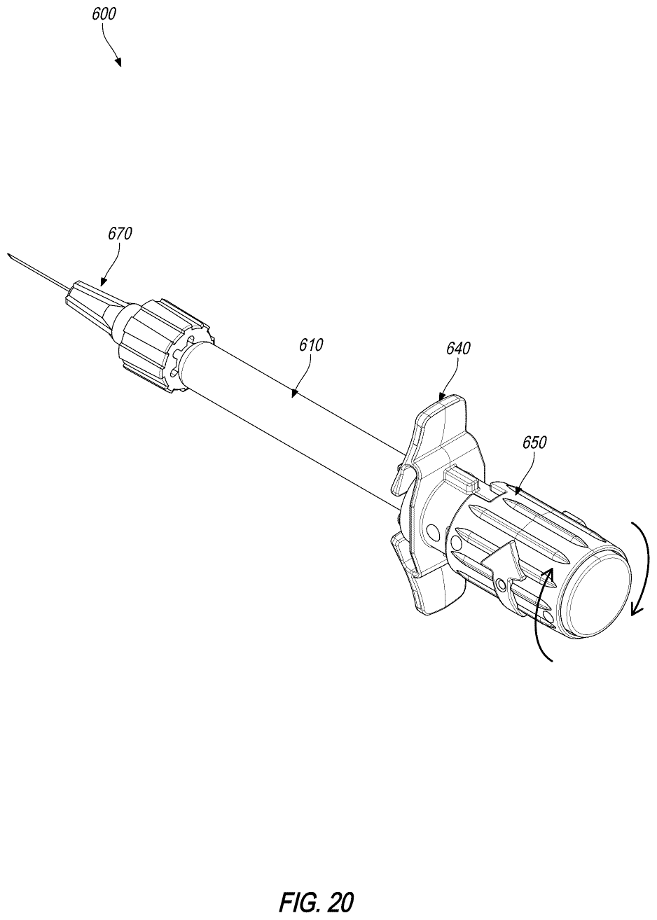

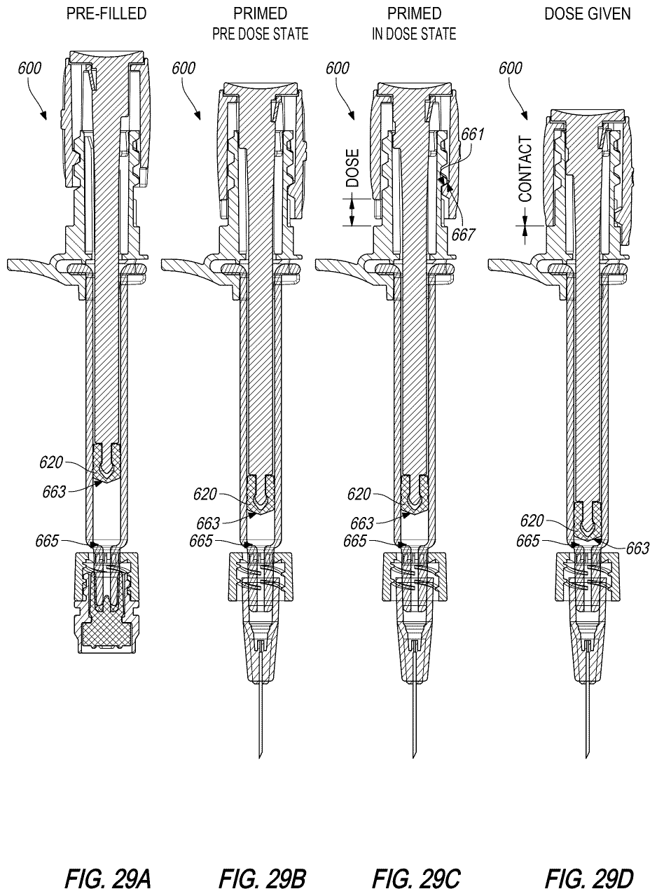

The shroud 650 defines a shroud distally facing surface 651 at a distal end thereof. The shroud distally facing surface 651 remains proximal of a proximally facing surface 645 of the flange elongate member 644 to when the shroud 650 is in a first longitudinal position on the finger flange 640 and a first rotational state relative to the finger flange 640 (see e.g., FIGS. 19 and 29C). The shroud 650 also defines a slot 652 extending proximally from the distally facing surface 651 and configured to receive the flange elongate member 644 to allow distal movement of the shroud 650 distally beyond the first longitudinal position on the finger flange 640 when the shroud 650 is in a second rotational state (see e.g., FIGS. 20 and 29D) relative to the finger flange 640. The slot 652 has a distally facing surface 653. While two slots 652 are shown, alternative embodiments may have one slot or 3 or more slots. The shroud 650 also defines an external indicia of rotation direction 654. The shroud 650 has a knurled external surface to facilitate manual rotation thereof.

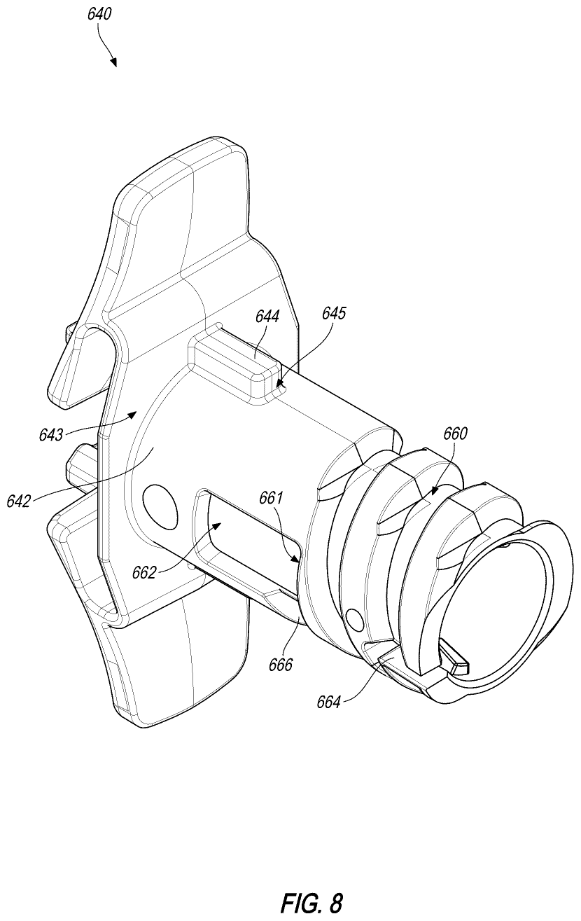

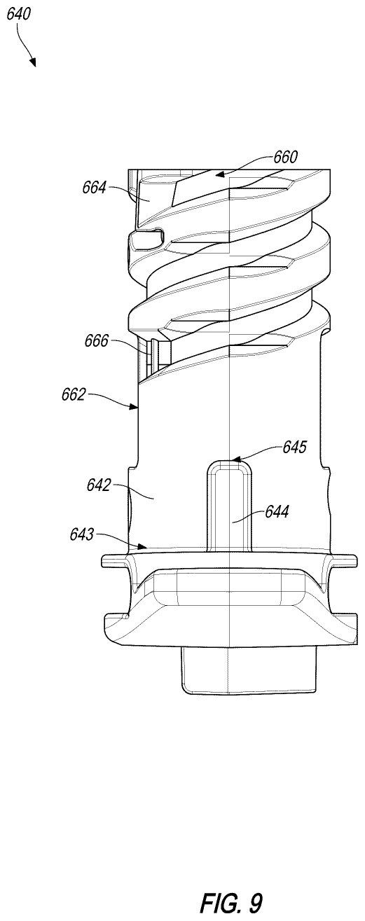

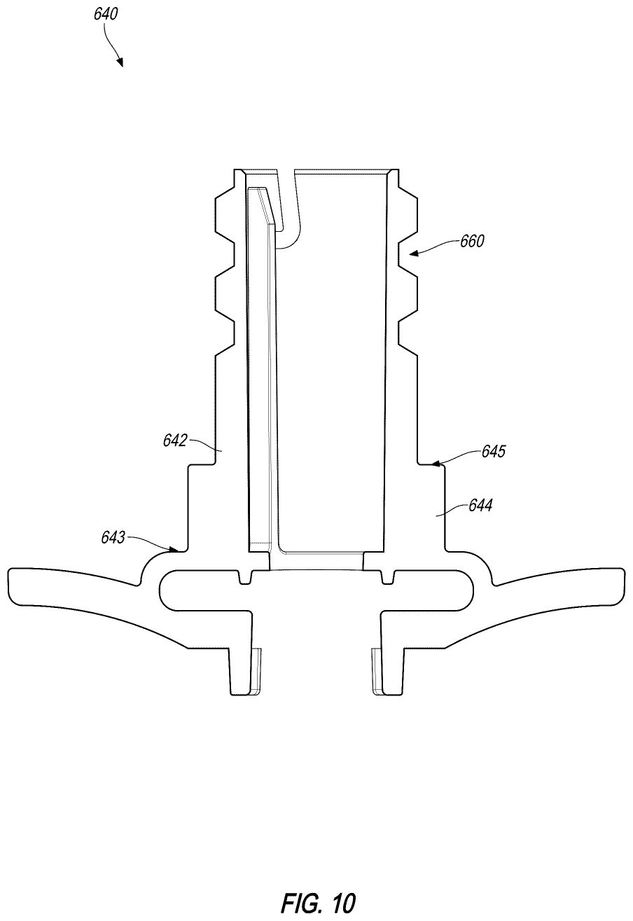

FIGS. 8, 9, and 10 are perspective, side, and longitudinal cross-sectional views depicting a finger flange 640 for use with systems for injecting, such as the system 600 depicted in FIGS. 6 and 7, according to some embodiments. The finger flange 640 defines a flange proximally facing surface 643 and a proximally extending member 642, which has a pair of external threads 660 defined thereon. The external threads 660 are double lead threads with about a 5 mm separation there between. The proximally extending member 642 also defines a pair of flange elongate members 644, each having a flange elongate member proximally facing surface 645. The proximally extending member 642 further defines a pair of distally extending external longitudinal slot 662 at respective distal ends of the external threads 660. Moreover, the proximally extending member 642 defines a proximal bump 664 adjacent both proximal ends of the external threads 660 and a pair of radial bumps 666 adjacent respective distal ends of the external threads 660. The radial bumps 666 separate the distal ends of the external threads 660 from the proximal ends of the longitudinal slots 662. In some embodiments, the proximally extending member may define a pair of proximal bumps adjacent respective proximal ends of the external threads.

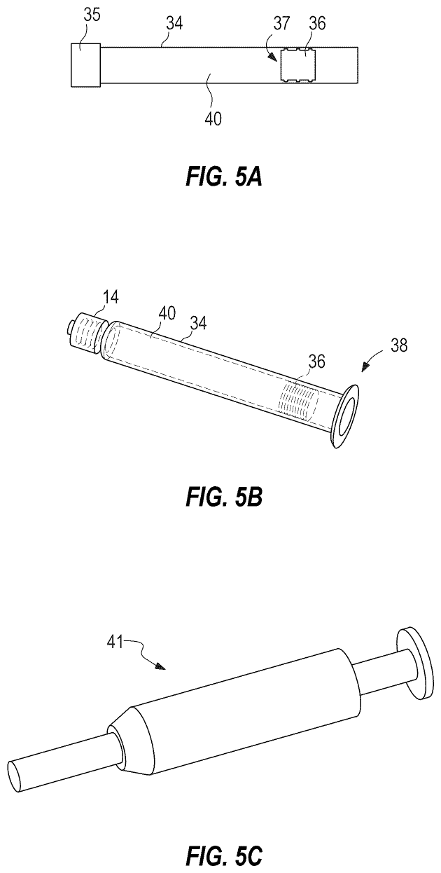

While the threaded connection illustrated in the embodiment herein is a double-lead or 2-start thread, the threads may be a single lead or have 3 or more leads. The thread lead or pitch or distance traveled in one thread revolution may be between 2 and 10 mm. In a preferred embodiment, the thread lead is 8.57 mm. The threaded connection may be configured with a thread lead or pitch that makes the thread self-locking, where the coefficient of friction between the finger flange and the shroud is greater than or equal to the tangent of the lead angle of the thread. Where the lead angle of the thread is the inverse tangent of the lead divided by TT times the thread diameter. In a self-locking thread, a proximally directed force that the stopper member exerts on the plunger member and the intercoupled shroud during use, manufacturing, or shipment will not rotate and/or translate the threaded shroud and intercoupled plunger member in a proximal direction. During terminal sterilization of a filled and closed syringe body, with the finger flange attached, pressure may build up in the medicine chamber due to the temperature increase. This pressure may cause a proximally directed force to be applied to the stopper member, urging the stopper to move proximally. It is desired not to allow the stopper to move more than the distance between the sealing ribs of the stopper member (see e.g., FIG. 5A) to prevent the exposure of potentially non-sterile surfaces of the stopper member and/or syringe body to the injectable fluid. The self-locking threaded connection between the finger flange and the shroud, and the intercoupled plunger member and stopper member prevents the movement of the stopper member proximally until the temperature of the prefilled syringe body is reduced and the pressure in the syringe interior is reduced. Additionally, during air shipment the filled, stoppered, and assembled syringe bodies may experience a drop in atmospheric pressure external to the syringe bodies. With a drop in external pressure, the injectable fluid and any entrained air bubbles in the syringe body may expand and apply a proximally directed force on the stopper member. It is desired not to allow the stopper member to move more than the distance between the sealing ribs of the stopper member to prevent the exposure of potentially non-sterile surfaces of the stopper member and/or syringe body to the injectable fluid. The self-locking threaded connection between the finger flange and shroud and the intercoupled plunger member and stopper member prevents the movement of the stopper member proximally until the external atmospheric pressure is returned to an amount similar to the pressure in the syringe interior.

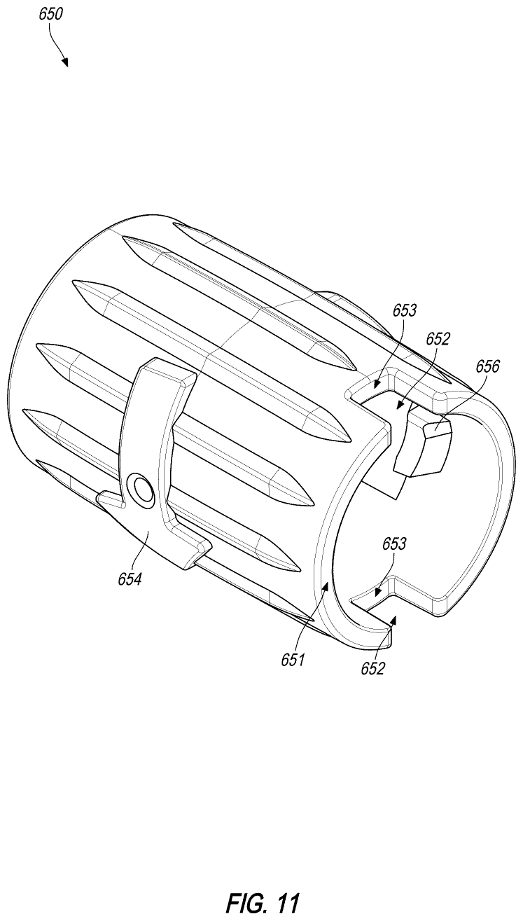

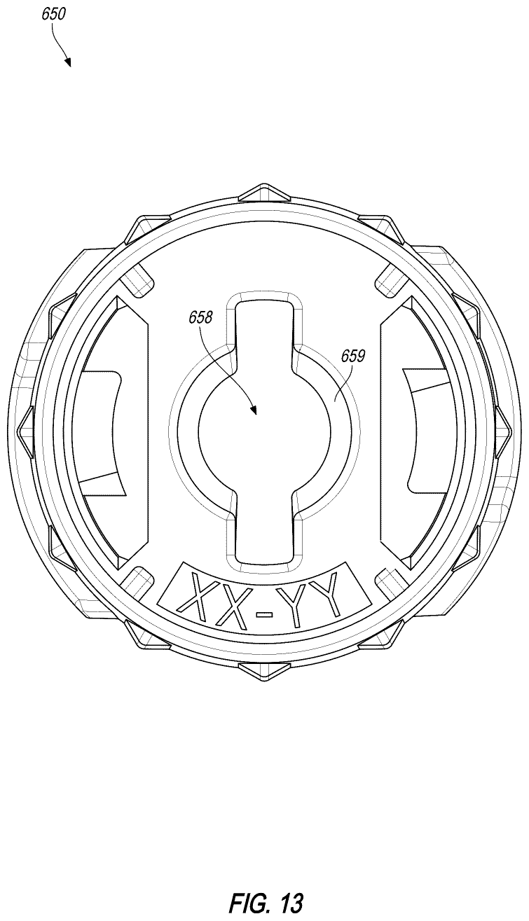

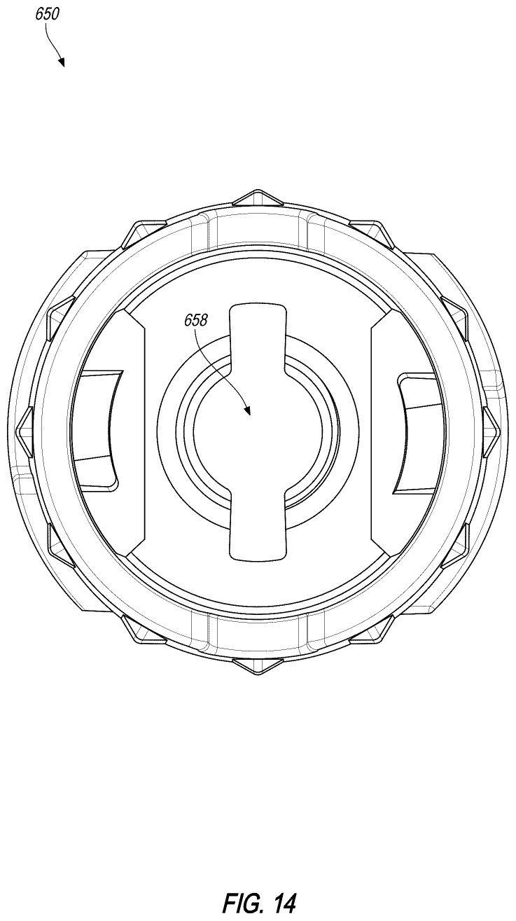

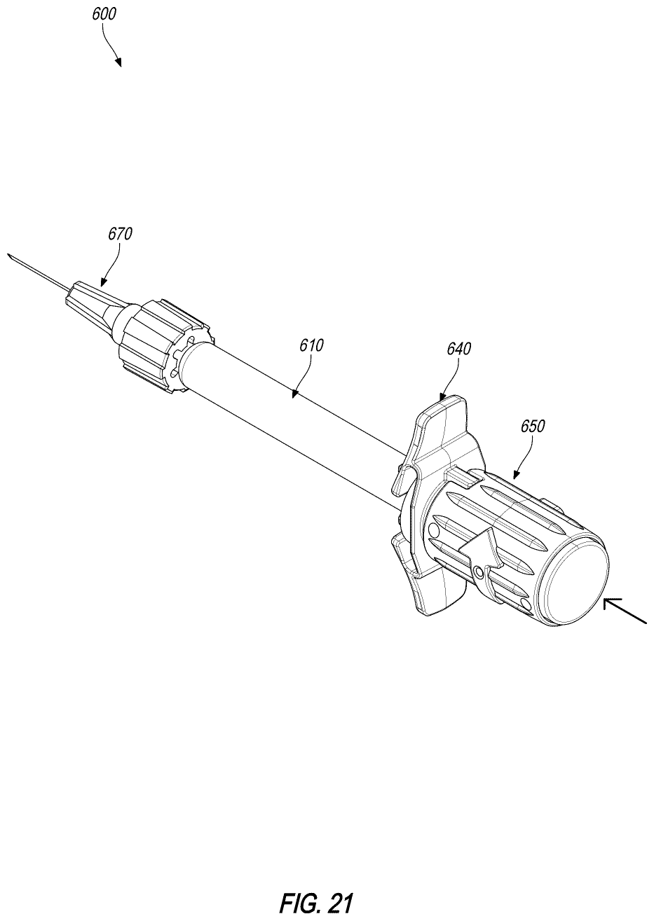

FIGS. 11, 12, 13, and 14 are perspective, longitudinal cross-sectional, bottom, and top views depicting a shroud 650 for use with systems for injecting, such as the system 600 depicted in FIGS. 6 and 7, according to some embodiments. The shroud 650 defines a distally facing surface 651 at a distal end thereof, and a pair of slots 652 extending proximally from the distally facing surface 651. The slots 652 are configured to receive the corresponding pair of flange elongate members 644 to allow distal movement of the shroud 650 relative to the finger flange 640 (see e.g., FIGS. 20 and 21). Each slot 652 has a distally facing surface 653. The shroud 650 also defines a pair of external indicia of rotation direction 654. The shroud 650 has a knurled external surface to facilitate manual rotation thereof. The shroud also defines a pair of inwardly extending members 656 configured to be disposed in respective external threads 660 in the proximally extending member 642 of the finger flange 640. The inwardly extending members 656 are also configured to travel along the external threads 660 with rotation of the shroud 650 relative to the finger flange 640. The inwardly extending members 656 are further configured to travel along the distally extending external longitudinal slots 662 with axial translation of the shroud 650 relative to the finger flange 640.

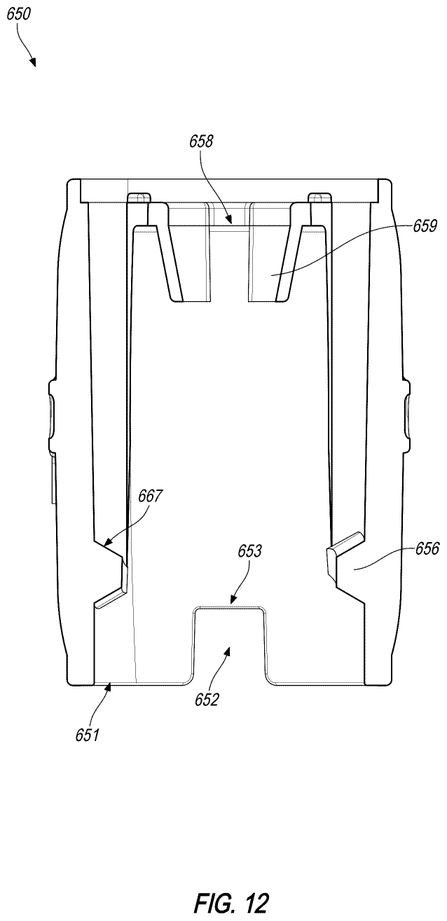

As shown in FIG. 12, the shroud 650 defines an opening 658 at a proximal end thereof. The shroud 650 also defines a pair of elastically deformable latching members 659 adjacent the opening 658. The pair of latching members 659 elastically deform outward to allowed the plunger member 630 to pass therethrough.

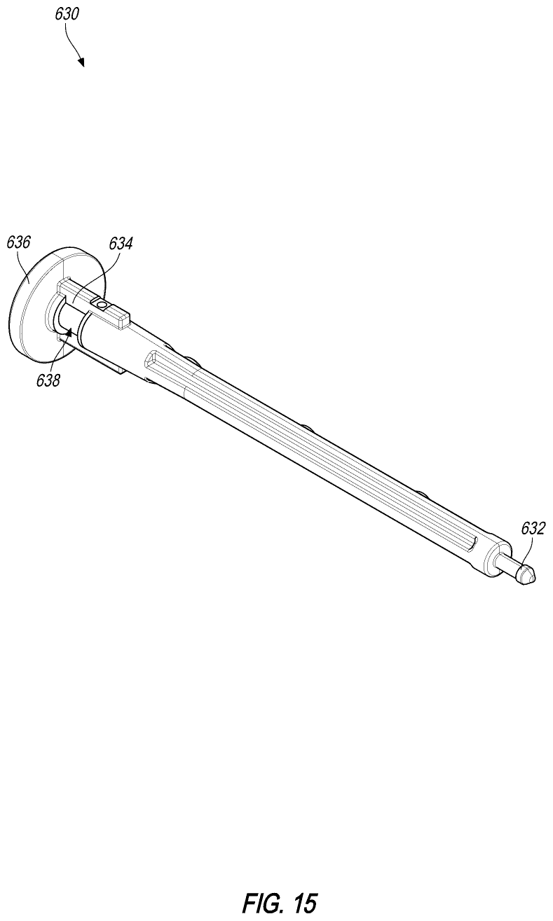

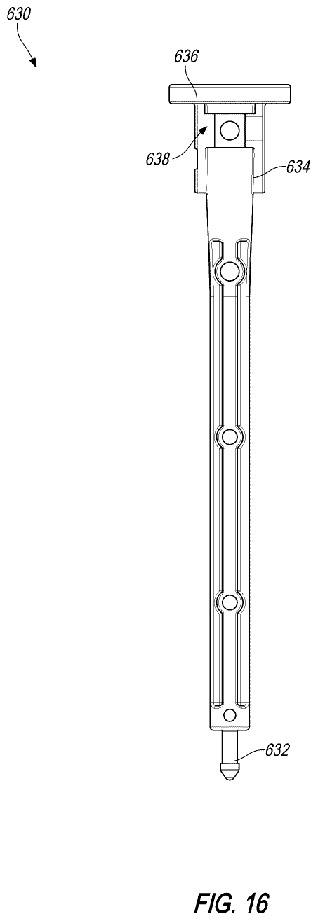

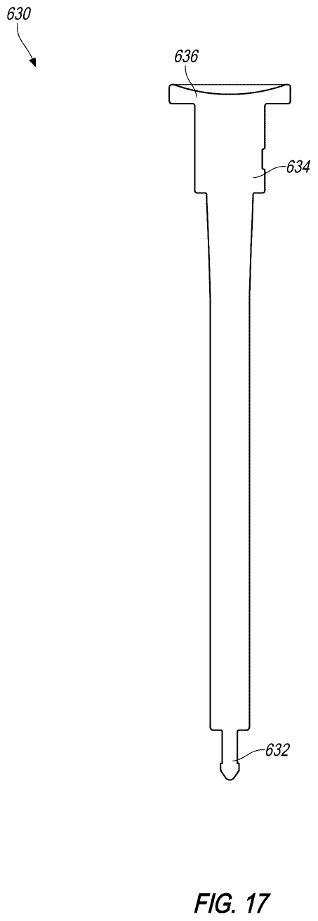

FIGS. 15, 16, and 17 are perspective, side, and longitudinal cross-sectional views depicting a plunger member 630 for use with systems for injecting, such as the system 600 depicted in FIGS. 6 and 7, according to some embodiments. The plunger member 630 defines a pair of grooves 638 adjacent a thumbpad 636. The pair of grooves 638 are configured to couple the plunger member 630 to the shroud 650. The pair of latching members 659 on the shroud 650 are configured to interfere with the pair of grooves 638 to removably couple the plunger member 630 to the shroud 650. The plunger member 630 may be rotatably and translationally coupled to the shroud 650. The plunger member 630 also defines a pair of elongate member 634 outwardly extending from an external surface thereof. The plunger member 630 further defines a distal end interference type connector 632 for a stopper member (not shown). In other embodiments, the distal end of the plunger member may have a screw type connector to couple the plunger member to the stopper member or a flat end that only pushes stopper members distally without the ability to pull stopper members proximally.

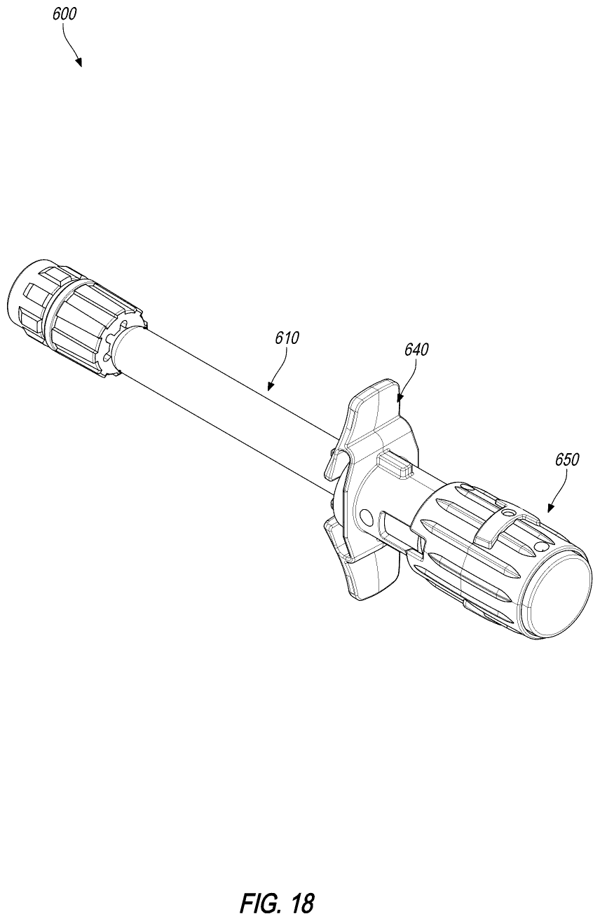

FIGS. 18, 19, 20, and 21 are perspective views depicting a system 600 for injecting in various configurations according to some embodiments. In FIG. 18, the system 600 is in a transport configuration in which the plunger member 630 and the shroud 650 are in a proximal most position. Note that a needle assembly 670 has been coupled to the syringe body 610 between FIGS. 18 and 19. Rotating the shroud 650 and the plunger member 630 coupled thereto relative to the finger flange 640 advances both the shroud 650 and the plunger member 630 and the stopper 620 (see FIGS. 29A to 29D) distally relative to the finger flange 640 transforms the system 600 into an almost primed/injection ready configuration depicted in FIG. 19.

During manufacturing, shipping, and storage prior to use the stopper 620 may experience additional initial break loose friction also known as stiction with the syringe body 610. This stiction results in an increased amount of axial force required to break the stopper 620 free from the syringe body 610 during priming. This stiction may also be increased by the desire to limit silicone oil lubrication on the inside of the syringe body 610 for ophthalmological and or intravitreal injections. The priming mechanism described herein provides a mechanical advantage to aide in breaking loose the stopper during priming. In particular, the shroud projection 656 is configured to engage the helical grooves/thread 660 of the finger flange 640 providing a threaded connection between the shroud 650 and the finger flange 640. This threaded connection provides a mechanical advantage to the user allowing the stopper 620 to be freed from the initial stiction with a break loose force applied tangential to the outside diameter of the shroud 650 by the user to break loose the stopper 620 from the syringe body 610. As the plunger member 630 is advanced distally relative to the finger flange 640 the system 600 is positioned in an upward orientation to allow an advancing stopper member (not shown) to eject air from a syringe interior (not shown).

Further rotation of the shroud 650 and the plunger member 630 coupled thereto relative to the finger flange 640 transforms the system 600 into a primed/injection ready configuration depicted in FIG. 20 by advancing the inwardly extending members 656 over the radial bumps 666. When the inwardly extending members 656 are advanced over the radial bumps 666, the interaction between the inwardly extending member 656 and radial bumps 666 provides a tactile and/or audible indicator that the system 600 has transitioned to the primed/injection ready configuration depicted in FIG. 20. In this configuration a proximally facing surface 667 (see FIGS. 12, 24, and 29C) of the inwardly extending members 656 transitions from following the helical surfaces of the external threads 660 to following a generally circumferential surface 661 to set an axial starting position of the shroud 650 and the intercoupled plunger member 630 and stopper member 620 in the primed/injection ready configuration. In the primed/injection ready configuration, the slots 652 on the shroud 650 are aligned with the proximal facing surfaces 645 of the flange elongate members 644 to indicate to the user that the shroud 650 and plunger member 630 are aligned and in a configuration where the dose can be delivered to the patient.

Distally advancing the shroud 650 and the plunger member 630 coupled thereto relative to the finger flange 640 transforms the system 600 into an injected configuration depicted in FIG. 21. In the injected configuration, the proximally facing surfaces 645 of the flange elongate members 644 are configured to interfere with respective distally facing surfaces 653 of the slots 652 formed in the shroud 650 to limit distal movement of the shroud 650 relative to the finger flange 640. In the injected configuration, the advancing plunger member 630 has advanced the stopper member 620 distally to eject a portion of the injectable fluid in the syringe interior 612. The system 600 is configured to advance the stopper member 620 to nearly the bottom of the syringe body 610 to eject a portion of the injectable fluid that is less than a total amount of the injectable fluid in the syringe interior 612 to accommodate tolerances in various components of the system 600. In an alternative embodiment, the system may be configured with a plunger member which is long enough to push the stopper member all the way to the bottom of the syringe body to expel all of the injectable fluid in the syringe interior.

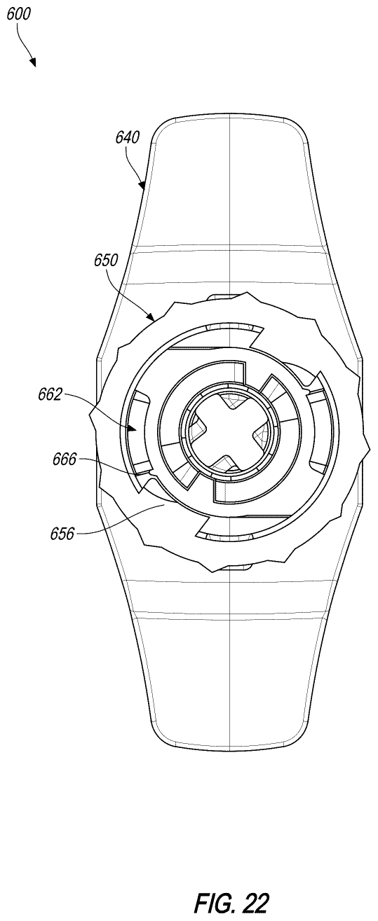

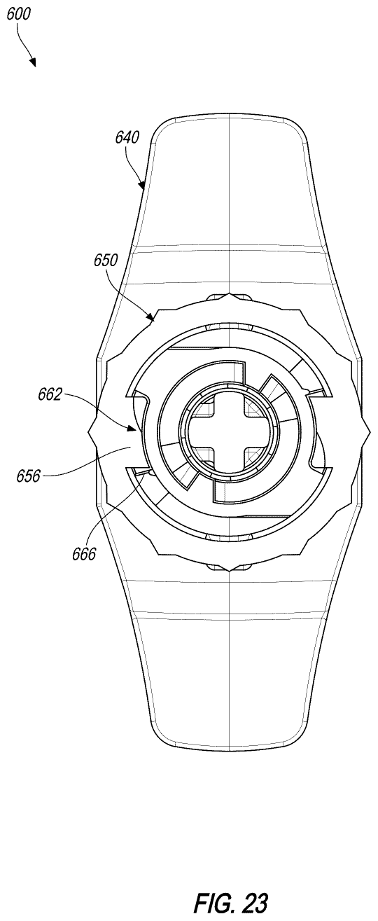

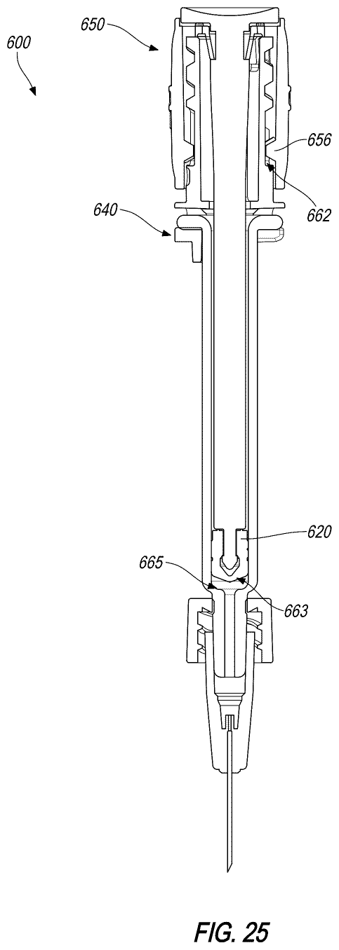

FIGS. 22, 23, 24, and 25 are transverse (FIGS. 22 and 23) and longitudinal (FIGS. 24 and 25) cross-sectional views depicting a system for injecting, such as the system 600 depicted in FIGS. 6 and 7, in various configurations according to some embodiments. In FIG. 22, the system 600 is in the almost primed/injection ready configuration depicted in FIG. 19, with the pair of inwardly extending members 656 of the shroud 650 in contact with, but not past, respective radial bumps 666 of the flinger flange 640. In FIGS. 23 and 24, the system 600 has been transformed, by rotation of the shroud 650 and plunger member 630 relative to the finger flange 640 into the primed/injection ready configuration where the proximally facing surfaces 667 of the inwardly extending members 656 of the shroud 650, and the distal circumferential surface 661 of the finger flange external thread 660 are in contact, to define the dose start position of the shroud 650 and the intercoupled plunger member 630 and stopper member 620. In FIG. 25, distal movement of the shroud 650 and plunger member 630 relative to the finger flange 640, transforms the system into the injected configuration depicted in FIG. 21, with the shroud 650 and the plunger member 630 in the distal most position and the injectable fluid expelled from the syringe body 610 through the needle.

In FIGS. 19 and 22, the pair of inwardly extending members 656 of the shroud 650 are in contact with, but not past, respective radial bumps 666 of the flinger flange 640. The extending members 656 of the shroud 650, the radial bumps 666, and the pair of external threads 660 of the finger flange 640 are configured to limit distal movement of the shroud 650 to a first longitudinal position on the finger flange 640 in the almost primed/injection ready configuration. In FIG. 20, the shroud 650 is rotated to move the pair of inwardly extending members 656 over the radial bumps 666 and into the respective distally extending external longitudinal slots 662. In FIG. 20, as the shroud 650 is rotated until the slots 652 extending proximally from the distally facing surface 651 of the shroud 650 align with respective flange elongate members 644 to transforms the system 600 to the primed/injection ready configuration depicted in FIG. 20. Moving the shroud 650 distally relative to the finger flange 640 until interference between the distally facing surface 653 of the slot 652 formed on the shroud 650 and the proximally facing surface 645 on the flange elongate members 644 transforms the system 600 to the injected configuration depicted in FIG. 21. In the injected configuration depicted in FIGS. 21, 23, and 25, the pair of inwardly extending members 656 are not in contact with respective distal ends of the distally extending external longitudinal slots 662.

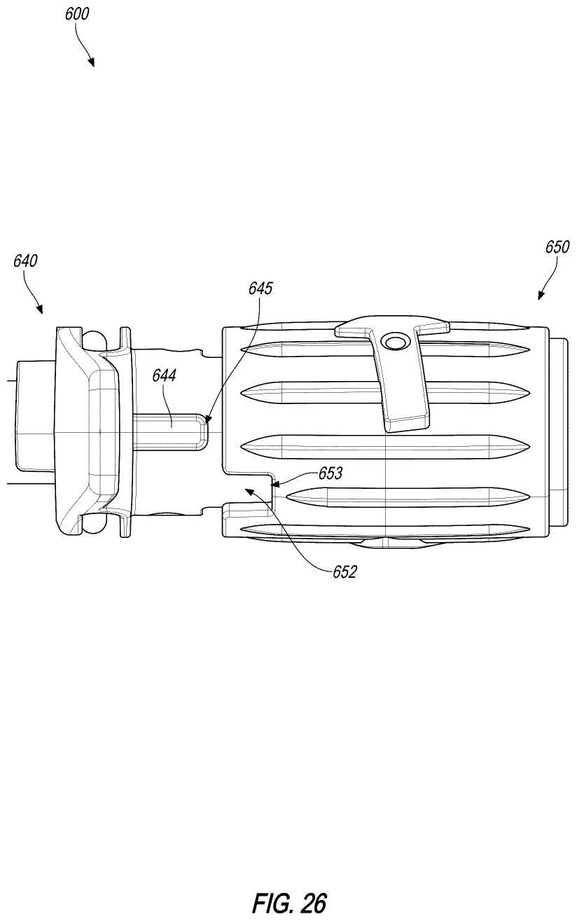

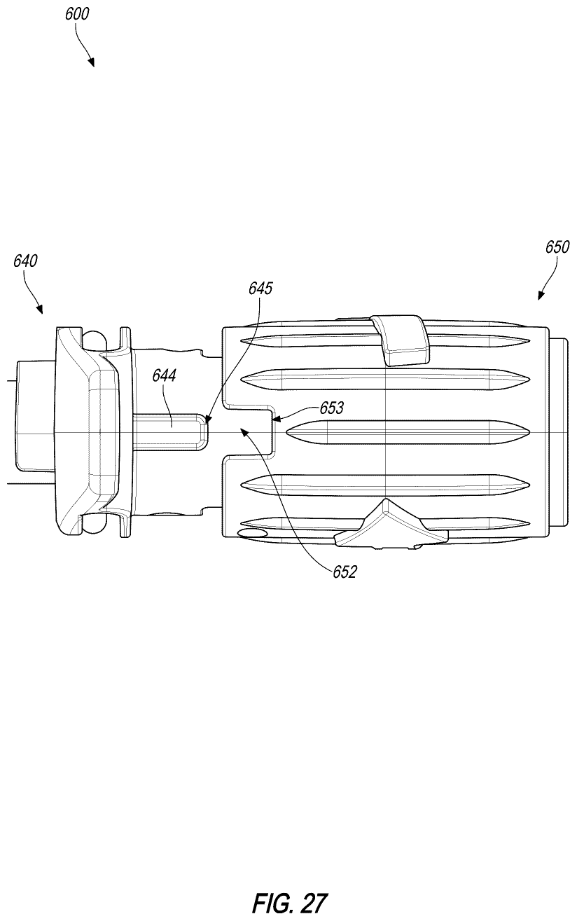

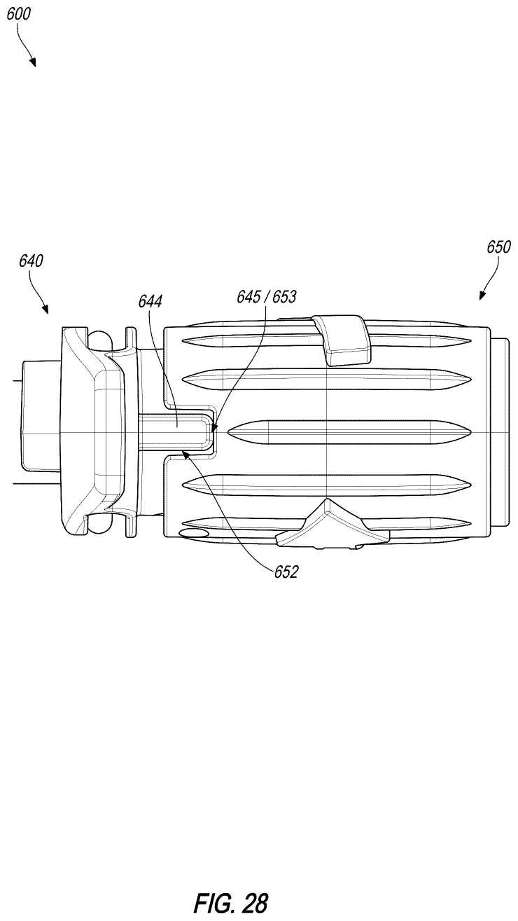

FIGS. 26, 27, and 28 are detailed side views depicting a shroud 650 and a finger flange 650 for use with systems for injecting, such as the system 600 depicted in FIGS. 6 and 7, in various configurations according to some embodiments. In FIG. 26, the system 600 is in the almost primed/injection ready configuration depicted in FIGS. 19 and 22. In FIGS. 19 and 22, the pair of inwardly extending members 656 of the shroud 650 are in contact with, but not past, respective radial bumps 666 of the flinger flange 640. The extending members 656 of the shroud 650, the radial bumps 666, and the pair of external threads 660 of the finger flange 640 are configured to limit distal movement of the shroud 650 to a first longitudinal position on the finger flange 640 in the almost primed/injection ready configuration. In FIGS. 24 and 27, the system 600 is in the primed/injection ready configuration depicted in FIG. 20, with the shroud 650 and the plunger member 630 in an intermediate position. In the primed/injection ready configuration, the slots 652 extending proximally from the distally facing surface 651 of the shroud 650 align with respective flange elongate members 644. In FIG. 28, the system 600 has been transformed, by distal movement of the shroud 650 and the plunger member 630 relative to the finger flange 640, to the injected configuration depicted in FIGS. 21, 23, and 25, with the shroud 650 and the plunger member 630 in a distal most position. In the injected configuration, the distally facing surfaces 653 of the slots 652 formed on the shroud 650 interferes with respective proximally facing surfaces 645 on the flange elongate members 644 to limit further distal movement of the shroud 650 relative to the finger flange 640.

FIGS. 29A, 29B, 29C, and 29D are longitudinal cross-sectional views depicting a system for injecting, such as the system 600 depicted in FIGS. 6 and 7, in various configurations according to some embodiments. In FIG. 29A, the system 600 is in the transport configuration depicted in FIG. 18. In FIG. 29B, the system 600 is in the almost primed/injection ready configuration depicted in FIGS. 19, 22, 24, and 26. In FIG. 29C, the system 600 is in the primed/injection ready configuration depicted in FIGS. 20 and 27. In FIG. 29D, the system 600 is in the injected configuration depicted in FIGS. 21, 23, 25, and 28. The plunger rod length is configured such that when contact is made between the distally facing surfaces 653 of the shroud 650 and the proximally facing surfaces 645 of the finger flange 640, the distal surface 663 of the stopper 620 is spaced from the syringe barrel bottom surface 665. This spacing allows the volume of the delivered dose to be independent of the manufacturing tolerance of the syringe barrel length, allowing enhanced precision and accuracy of the delivered dose volume.

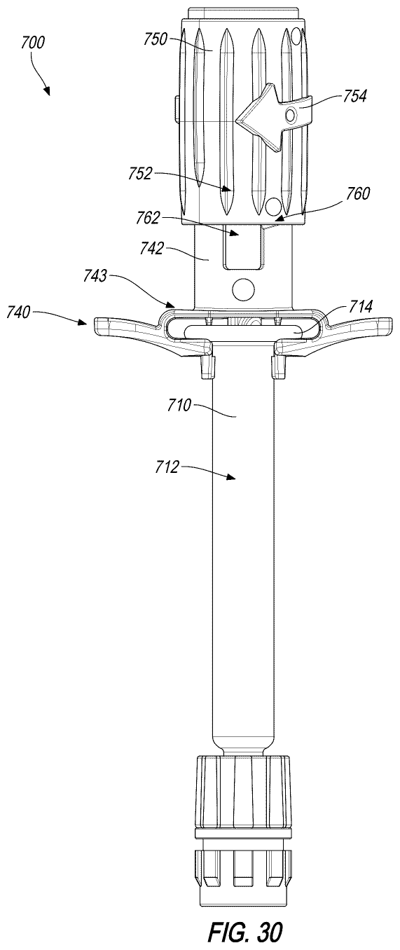

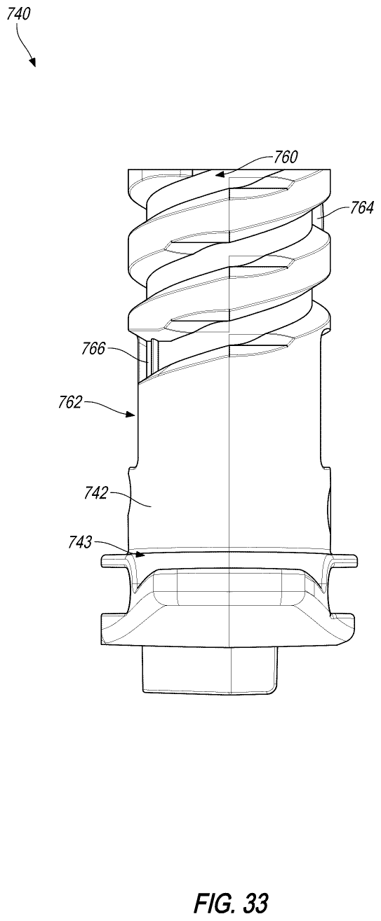

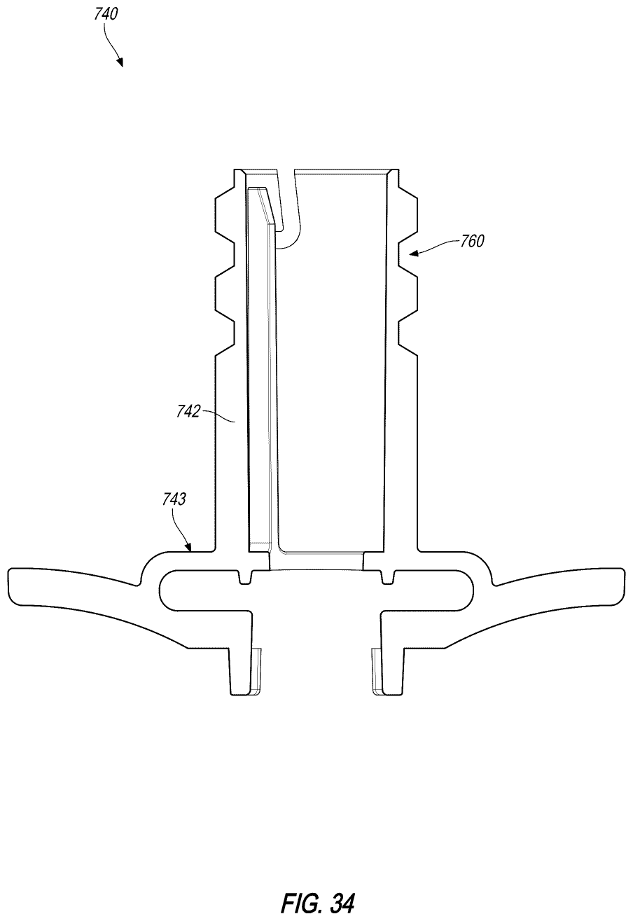

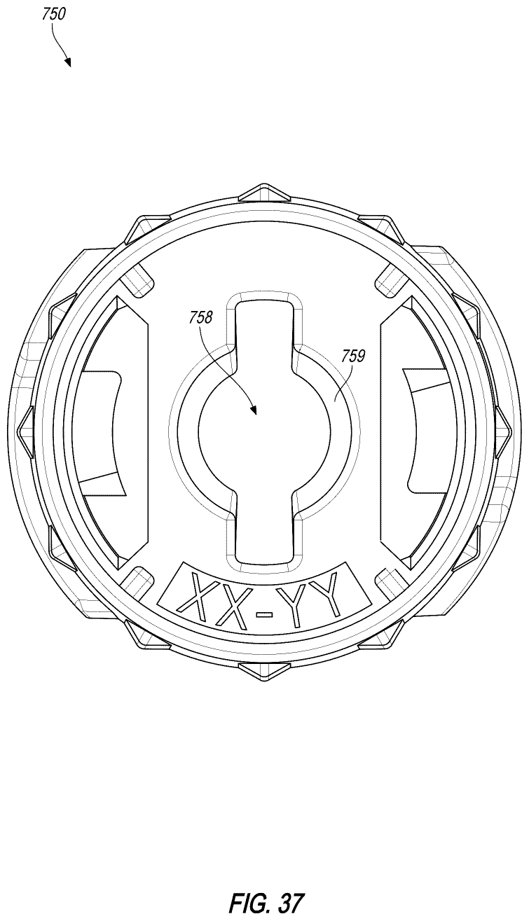

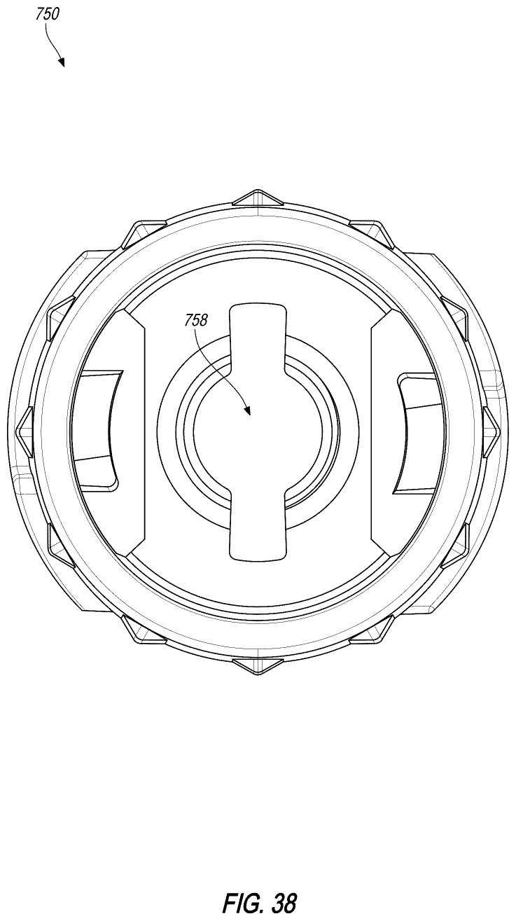

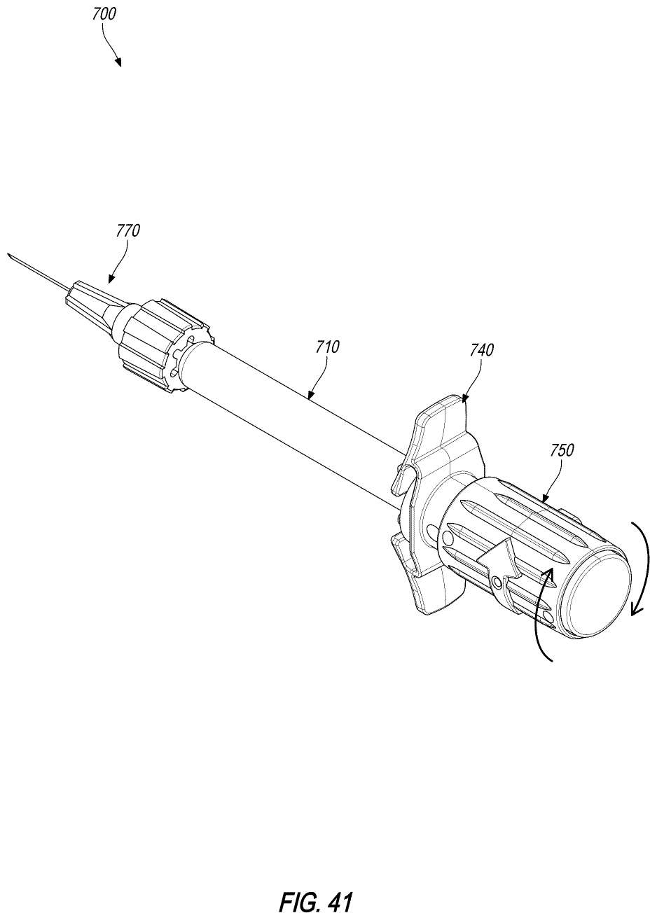

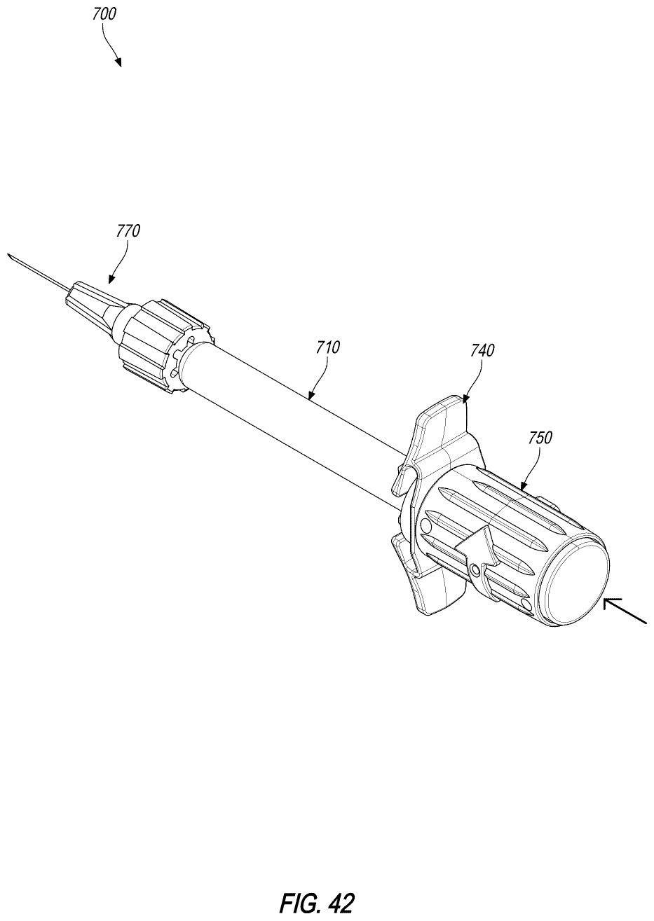

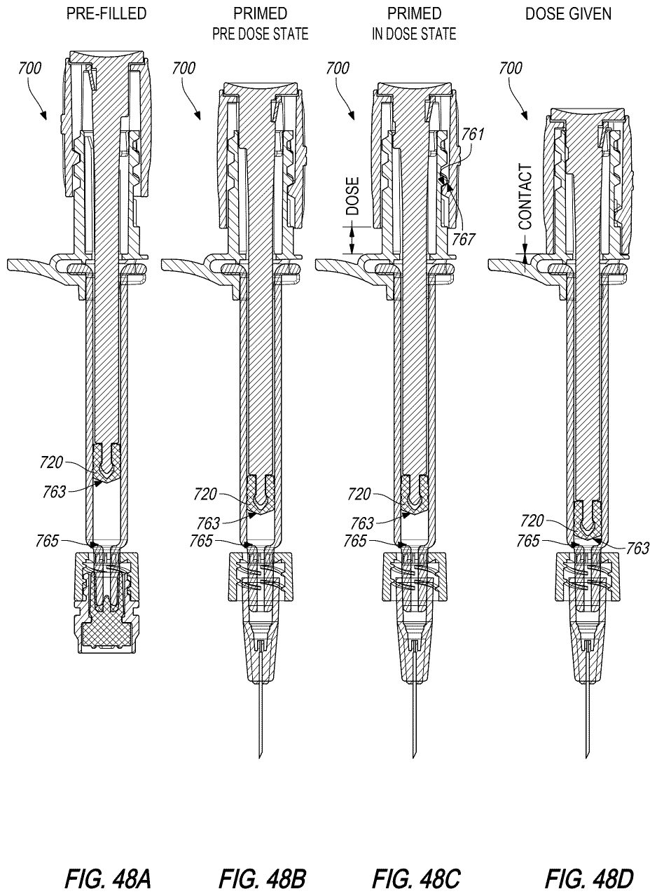

FIGS. 30 to 48D depict various aspects of a system/syringe assembly 700 for injecting according to some embodiments. The system 700 depicted in FIGS. 30 to 48D is similar to the system 600 depicted in FIGS. 6 to 29D, and function in a similar manner to perform an injection. The main difference between the two systems 600, 700 is that the system 700 depicted in FIGS. 30 to 48D does not have the pair of flange elongate members 644 on the flinger flange 640 and the corresponding pair of slots 652 on the shroud 650, which are found in the system 600 depicted in FIGS. 6 to 29D. As such, in the primed/injection ready configuration (see e.g., FIGS. 41 and 48C), distal movement of the shroud 750 relative to the finger flange 740 is limited to a first longitudinal position by interference between a pair of inwardly extending members 756 and corresponding external threads 760 in the proximally extending member 742 of the finger flange 740. In the injected configuration (see e.g., FIGS. 42 and 48D), a shroud distally facing surface 751 is configured to interfere with a flange proximally facing surface 743 to limit distal movement of the shroud 750 relative to the finger flange 740 to a second longitudinal position. In the injected configuration, the pair of inwardly extending members 756 are not in contact with respective distal ends of the distally extending external longitudinal slots 762.

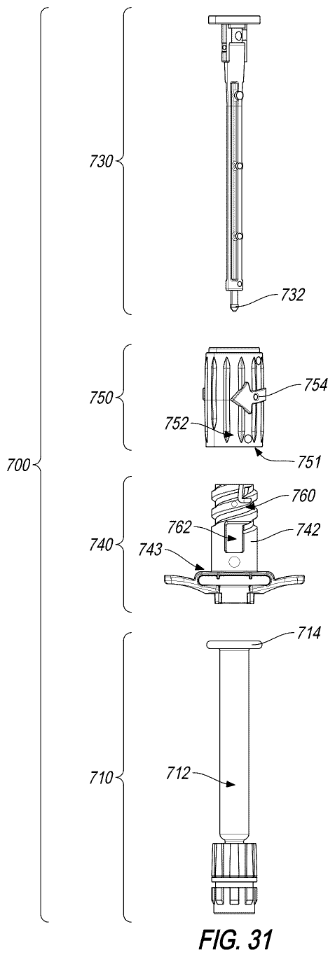

FIG. 30 is a front view depicting a system/syringe assembly 700 for injecting according to some embodiments. FIG. 31 is an exploded view depicting the system/syringe assembly 700 for injecting according to some embodiments. The system 700 includes a syringe body 710 having proximal and distal ends, a syringe interior 712, and a syringe flange 714 at the proximal end thereof. A stopper member 720 is disposed in the syringe interior 712 to form a fluid chamber therein. The syringe interior/fluid chamber 712 contains an injectable fluid (not shown) and some air. A plunger member 730 is partially disposed in the syringe interior 712. The plunger member 730 has a distal end 732 disposed adjacent to the stopper member 720. The system 700 also includes a finger flange 740 coupled to the syringe flange 712. The system 700 further includes a shroud 750 coupled to the plunger member 730 and movable relative to the finger flange 740.

The plunger member 730 is rotatably and translationally coupled to the shroud 750. While the distal end 732 of the plunger member 730 of the system depicted in FIG. 31 has an interference type connector for a stopper member (not shown), in other embodiments the distal end of the plunger member may have a screw type connector to couple the plunger member to the stopper member or a flat end that only pushes stopper members distally without the ability to pull stopper members proximally.

The finger flange 740 defines a proximally extending member 742, which has a pair of external threads 760 defined thereon. The external threads 760 are double lead threads with about a 5 mm separation there between. The proximally extending member 742 also defines a pair of distally extending external longitudinal slot 762 at respective distal ends of the external threads 760. The finger flange 740 defines a flange proximally facing surface 743.

The shroud 750 defines a shroud distally facing surface 751 at a distal end thereof configured to interfere with flange proximally facing surface 743 to limit distal movement of the shroud 750 to a second longitudinal position on the finger flange 740 when the shroud 750 is in a first rotational state (see e.g., FIGS. 42 and 48D) relative to the finger flange 740. The shroud 750 also defines an external indicia of rotation direction 754. The shroud 750 has a knurled external surface to facilitate manual rotation thereof.

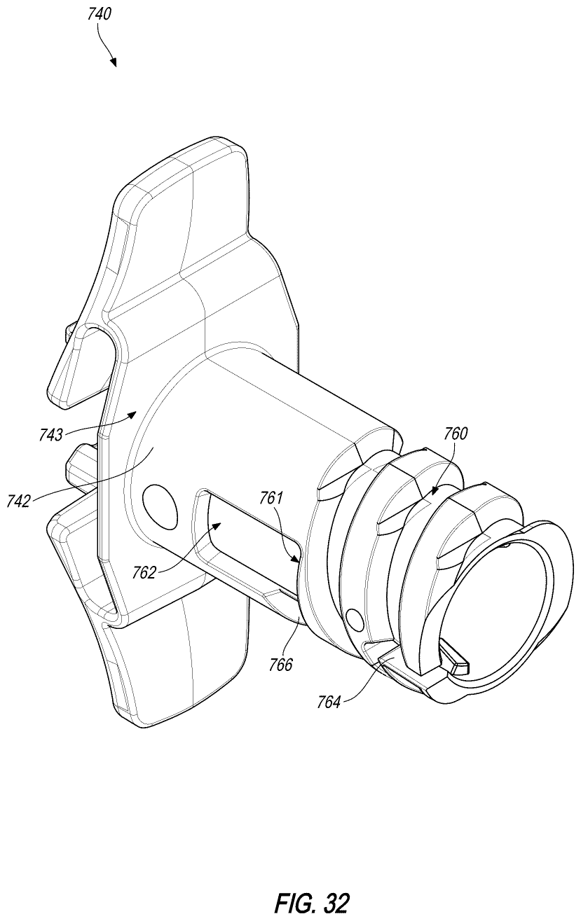

FIGS. 32, 33, and 34 are perspective, side, and longitudinal cross-sectional views depicting a finger flange 740 for use with systems for injecting, such as the system 700 depicted in FIGS. 30 and 31, according to some embodiments. The finger flange 740 defines a flange proximally facing surface 743 and a proximally extending member 742, which has a pair of external threads 760 defined thereon. The external threads 760 are double lead threads with about a 5 mm separation there between. The proximally extending member 742 also defines a pair of distally extending external longitudinal slot 762 at respective distal ends of the external threads 760. The proximally extending member 742 further defines a pair of proximal bumps 764 adjacent respective proximal ends of the external threads 760 and a pair of radial bumps 766 adjacent respective distal ends of the external threads 760. The radial bumps 766 separate the distal ends of the external threads 760 from the proximal ends of the longitudinal slots 762.

While the threaded connection illustrated in the embodiment herein is a double-lead or 2-start thread, the threads may be a single lead or have 3 or more leads. The thread lead or pitch or distance traveled in one thread revolution may be between 2 and 10 mm. In a preferred embodiment, the thread lead is 8.57 mm. The threaded connection may be configured with a thread lead or pitch that makes the thread self-locking, where the coefficient of friction between the finger flange and the shroud is greater than or equal to the tangent of the lead angle of the thread. Where the lead angle of the thread is the inverse tangent of the lead divided by TT times the thread diameter. In a self-locking thread, a proximally directed force that the stopper member exerts on the plunger member and the intercoupled shroud during use, manufacturing, or shipment will not rotate and/or translate the threaded shroud and intercoupled plunger member in a proximal direction. During terminal sterilization of a filled and closed syringe body, with the finger flange attached, pressure may build up in the medicine chamber due to the temperature increase. This pressure may cause a proximally directed force to be applied to the stopper member, urging the stopper to move proximally. It is desired not to allow the stopper to move more than the distance between the sealing ribs of the stopper member (see e.g., FIG. 5A) to prevent the exposure of potentially non-sterile surfaces of the stopper member and/or syringe body to the injectable fluid. The self-locking threaded connection between the finger flange and the shroud, and the intercoupled plunger member and stopper member prevents the movement of the stopper member proximally until the temperature of the prefilled syringe body is reduced and the pressure in the syringe interior is reduced. Additionally, during air shipment the filled, stoppered, and assembled syringe bodies may experience a drop in atmospheric pressure external to the syringe bodies. With a drop in external pressure, the injectable fluid and any entrained air bubbles in the syringe body may expand and apply a proximally directed force on the stopper member. It is desired not to allow the stopper member to move more than the distance between the sealing ribs of the stopper member to prevent the exposure of potentially non-sterile surfaces of the stopper member and/or syringe body to the injectable fluid. The self-locking threaded connection between the finger flange and shroud and the intercoupled plunger member and stopper member prevents the movement of the stopper member proximally until the external atmospheric pressure is returned to an amount similar to the pressure in the syringe interior.

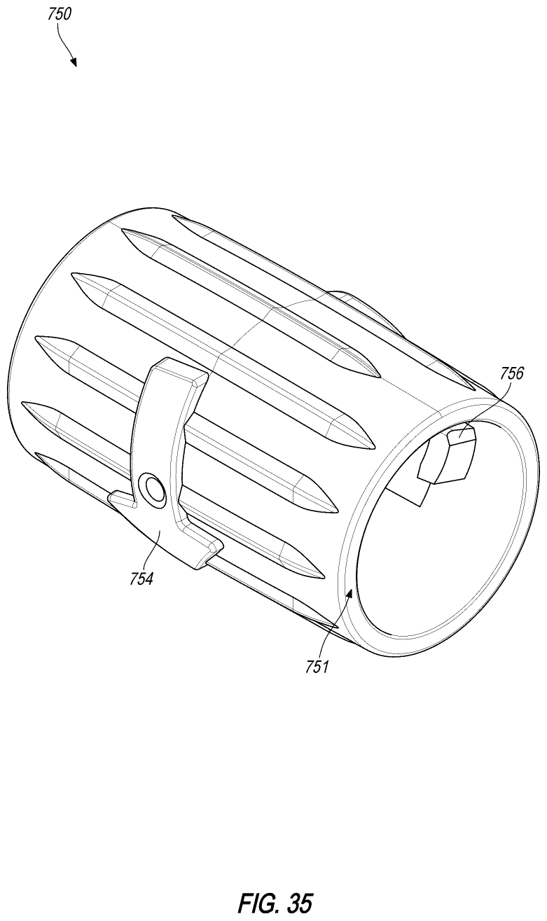

FIGS. 35, 36, 37, and 38 are perspective, longitudinal cross-sectional, bottom, and top views depicting a shroud 750 for use with systems for injecting, such as the system 700 depicted in FIGS. 30 and 31, according to some embodiments. The shroud 750 defines a distally facing surface 751 at a distal end thereof. The shroud 750 also defines a pair of external indicia of rotation direction 754. The shroud 750 has a knurled external surface to facilitate manual rotation thereof. The shroud also defines a pair of inwardly extending members 756 configured to be disposed in respective external threads 760 in the proximally extending member 742 of the finger flange 740. The inwardly extending members 756 are also configured to travel along the external threads 760 with rotation of the shroud 750 relative to the finger flange 740. The inwardly extending members 756 are further configured to travel along the distally extending external longitudinal slots 762 with axial translation of the shroud 750 relative to the finger flange 740. The inwardly extending members 756 are configured to interfere with the radial bumps 766 and external threads 760 to limit distal movement of the shroud 750 to a first longitudinal position on the finger flange 740 when the shroud 750 is in a first rotational state (see e.g., FIGS. 41 and 48C) relative to the finger flange 740.

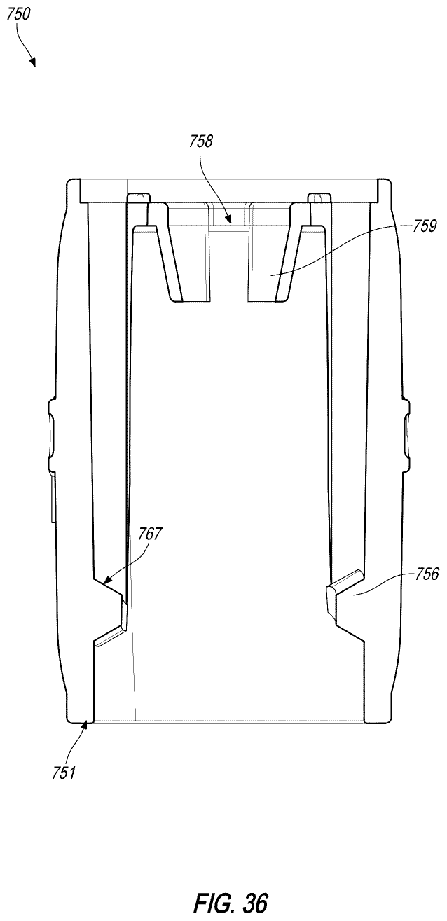

As shown in FIG. 36, the shroud 750 defines an opening 758 at a proximal end thereof. The shroud 750 also defines a pair of elastically deformable latching members 759 adjacent the opening 758. The pair of latching members 759 elastically deform outward to allowed the plunger member 730 to pass therethrough.

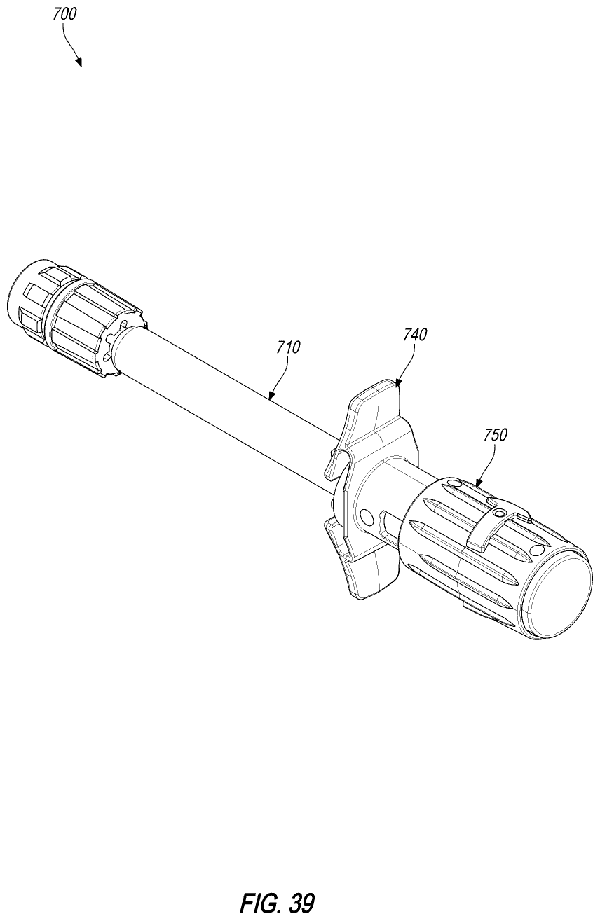

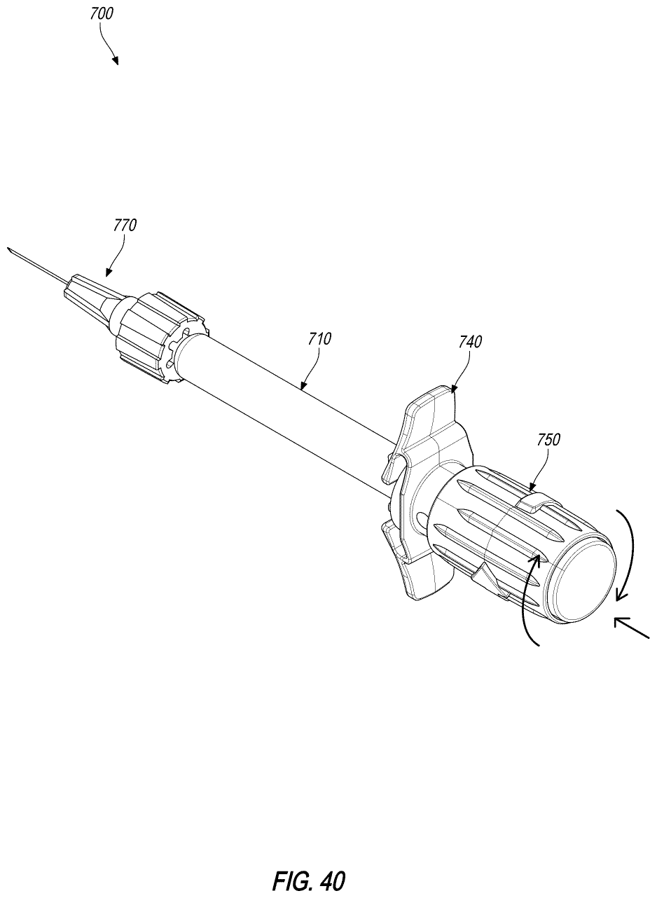

FIGS. 39, 40, 41, and 42 are perspective views depicting a system 700 for injecting in various configurations according to some embodiments. In FIG. 39, the system 700 is in a transport configuration in which the plunger member 730 and the shroud 750 are in a proximal most position. Note that a needle assembly 770 has been coupled to the syringe body 710 between FIGS. 39 and 40. Rotating the shroud 750 and the plunger member 730 coupled thereto relative to the finger flange 740 advances both the shroud 750 in the plunger member 730 and the stopper 720 distally relative to the finger flange 740 transforms the system 700 into an almost primed/injection ready configuration depicted in FIG. 40.

During manufacturing, shipping, and storage prior to use the stopper 720 may experience additional initial break loose friction also known as stiction with the syringe body 710. This stiction results in an increased amount of axial force required to break the stopper 720 free from the syringe body 710 during priming. This stiction may also be increased by the desire to limit silicone oil lubrication on the inside of the syringe body 710 for ophthalmological and or intravitreal injections. The priming mechanism described herein provides a mechanical advantage to aide in breaking loose the stopper during priming. In particular, the shroud projection 756 is configured to engage the helical grooves/threads 760 of the finger flange 740 providing a threaded connection between the shroud 750 and the finger flange 740. This threaded connection provides a mechanical advantage to the user allowing the stopper 720 to be freed from the initial stiction with a break loose force applied tangential to the outside diameter of the shroud 750 by the user to break loose the stopper 720 from the syringe body 710. As the plunger member 730 is advanced distally relative to the finger flange 740 the system 700 is positioned in an upward orientation to allow an advancing stopper member (not shown) to eject air from a syringe interior (not shown).

Further rotation of the shroud 750 and the plunger member 730 coupled thereto relative to the finger flange 740 transforms the system 700 into a primed/injection ready configuration depicted in FIG. 41 by advancing the inwardly extending members 756 over the radial bumps 766. When the inwardly extending members 756 are advanced over the radial bumps 766, the interaction between the inwardly extending member 756 and radial bumps 766 provides a tactile and/or audible indicator that the system 700 has transitioned to the primed/injection ready configuration depicted in FIG. 41. In the primed/injection ready configuration, proximally facing surfaces 767 of the inwardly extending members 756 of the shroud 750 and the circumferential surface 761 of the finger flange external thread 760 are in contact to define the dose start position of the shroud 750 and the intercoupled plunger member 730 and stopper member 720 (see e.g., FIGS. 36, 43, 48C).

Distally advancing the shroud 750 and the plunger member 730 coupled thereto relative to the finger flange 740 transforms the system 700 into an injected configuration depicted in FIG. 42. In the injected configuration, a proximally facing surfaces 743 of the finger flange 740 are configured to interfere with a distally facing surface 751 of the shroud 750 to limit distal movement of the shroud 750 relative to the finger flange 740. In the injected configuration, the advancing plunger member 730 has advanced the stopper member 720 distally to eject a portion of the injectable fluid in the syringe interior 712. The system 700 is configured to eject a portion of the injectable fluid that is less than a total amount of the injectable fluid in the syringe interior 712 to accommodate tolerances in various components of the system 700.

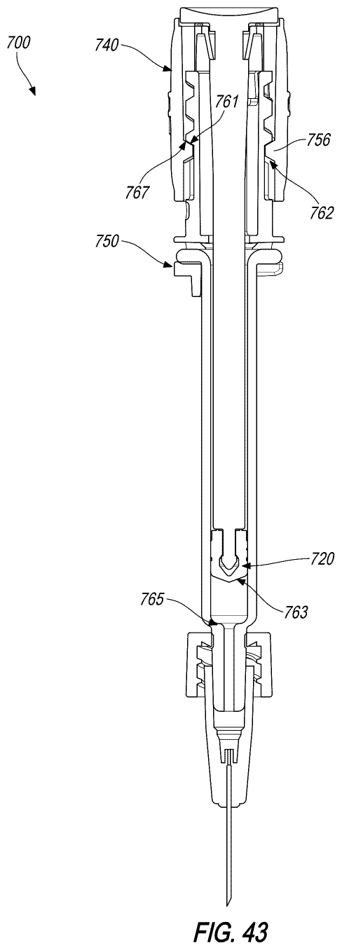

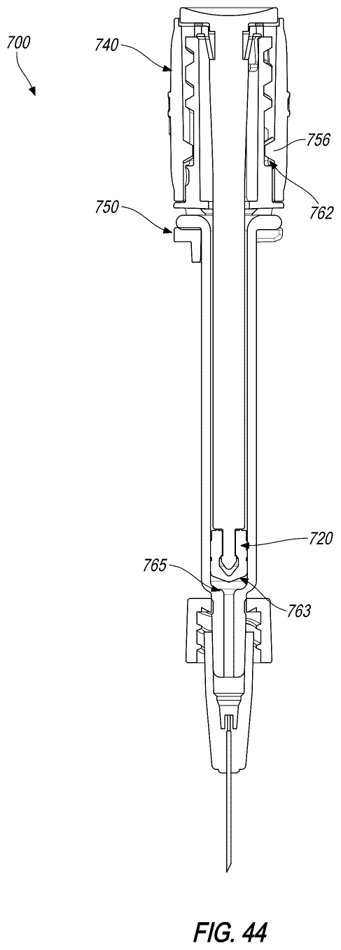

FIGS. 43 and 44 are longitudinal cross-sectional views depicting a system for injecting, such as the system 700 depicted in FIGS. 30 and 31, in various configurations according to some embodiments. In FIG. 43, the system 700 is in the almost primed/injection ready configuration depicted in FIG. 40, with the pair of inwardly extending members 756 of the shroud 750 in contact with, but not past, respective radial bumps 766 of the flinger flange 740. In FIG. 44, the system 700 has been transformed, by rotation of the shroud 750 and plunger member 730 relative to the finger flange 740 and distal movement of the shroud 750 and plunger member 730 relative to the finger flange 740, to the injected configuration depicted in FIG. 42, with the shroud 750 and the plunger member 730 in the distal most position.

In FIGS. 40 and 43, the inwardly extending members 756 interfere with the radial bumps 766 and external threads 760 to limit distal movement of the shroud 750 to a first longitudinal position on the finger flange 740 in the almost primed/injection ready configuration. In FIG. 41, the shroud 750 is rotated to move the pair of inwardly extending members 756 over the radial bumps 766 and into the respective distally extending external longitudinal slots 762, thereby transforming the system 700 to the primed/injection ready configuration depicted. Moving the shroud 750 distally relative to the finger flange 740 until interference between the shroud distally facing surface 751 and the flange proximally facing surface 743 transforms the system 700 to the injected configuration depicted in FIG. 42. In the injected configuration depicted in FIGS. 42 and 44, the pair of inwardly extending members 756 are not in contact with respective distal ends of the distally extending external longitudinal slots 762.

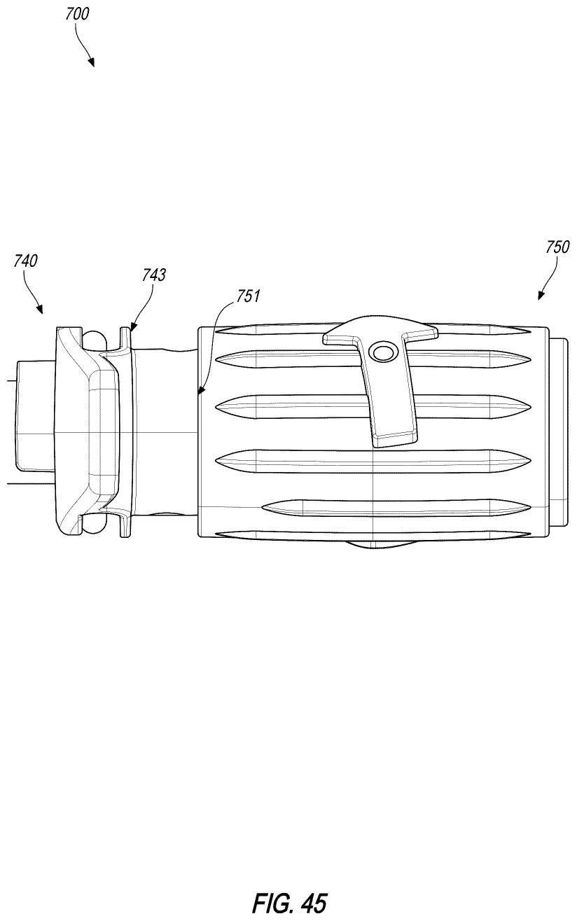

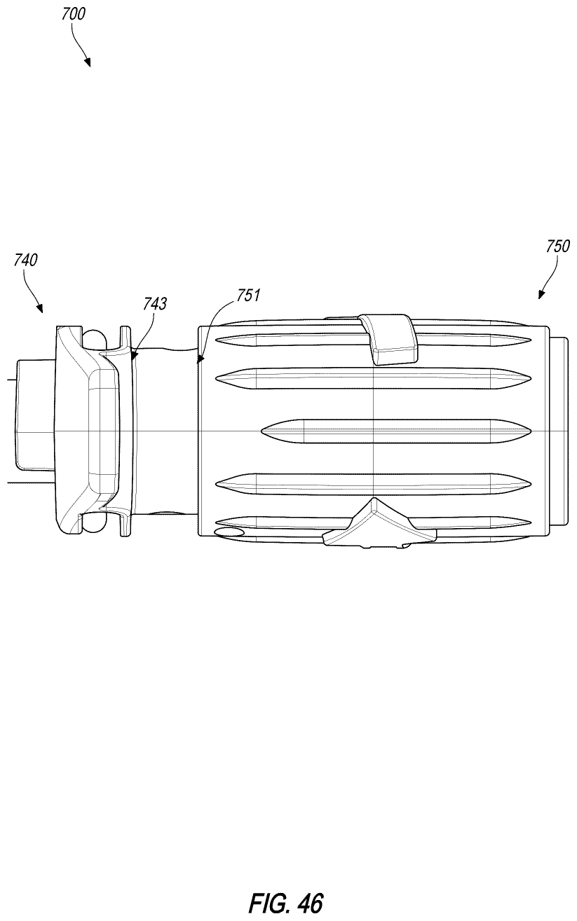

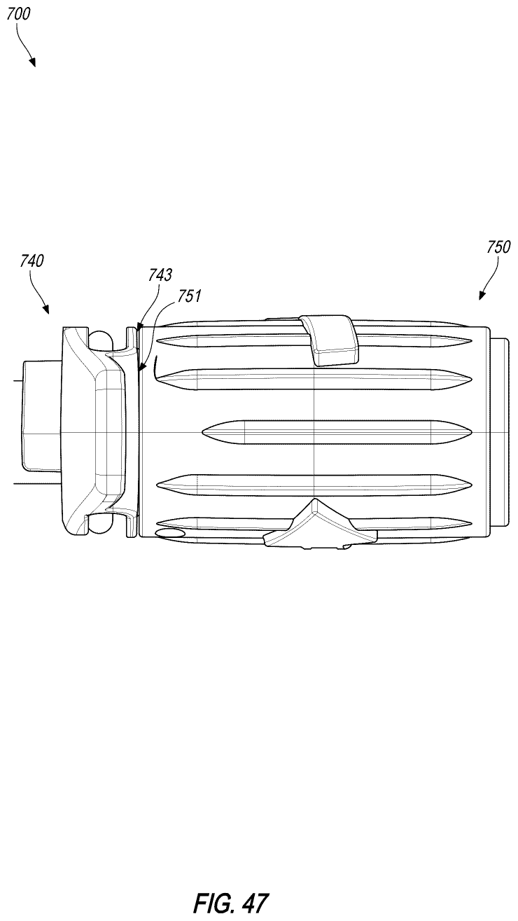

FIGS. 45, 46, and 47 are detailed side views depicting a shroud 750 and a finger flange 750 for use with systems for injecting, such as the system 700 depicted in FIGS. 30 and 31, in various configurations according to some embodiments. In FIG. 45, the system 700 is in the almost primed/injection ready configuration depicted in FIGS. 40 and 43. In the almost primed/injection ready configuration, the inwardly extending members 756 interfere with the radial bumps 766 and external threads 760 to limit distal movement of the shroud 750 to a first longitudinal position on the finger flange 740. In FIG. 46, the system 700 is in the primed/injection ready configuration depicted in FIG. 41, with the shroud 750 and the plunger member 730 in an intermediate position. In the primed/injection ready configuration, the inwardly extending members 756 have moved into the distally extending external longitudinal slots 762 at respective distal ends of the external threads 760. In FIG. 47, the system 700 has been transformed, by distal movement of the shroud 750 and the plunger member 730 relative to the finger flange 740, to the injected configuration depicted in FIGS. 42 and 44, with the shroud 750 and the plunger member 730 in a distal most position. In the injected configuration, the shroud distally facing surface 751 interferes with the flange proximally facing surface 743 to limit further distal movement of the shroud 750 relative to the finger flange 740.

FIGS. 48A, 48B, 48C, and 48D are longitudinal cross-sectional views depicting a system for injecting, such as the system 700 depicted in FIGS. 30 and 31, in various configurations according to some embodiments. In FIG. 48A, the system 700 is in the transport configuration depicted in FIG. 39. In FIG. 48B, the system 700 is in the almost primed/injection ready configuration depicted in FIGS. 40 and 43. In FIG. 48C, the system 700 is in the primed/injection ready configuration depicted in FIG. 41. In FIG. 48D, the system 700 is in the injected configuration depicted in FIGS. 42 and 44. The plunger rod length is configured such that when contact is made between the distally facing surfaces 751 of the shroud 750 and the proximally facing surfaces 743 of the finger flange 740, the distal surface 763 of the stopper 720 is spaced from the syringe barrel bottom surface 765. This spacing allows the volume of the delivered dose to be independent of the manufacturing tolerance of the syringe barrel length, allowing enhanced precision and accuracy of the delivered dose volume.

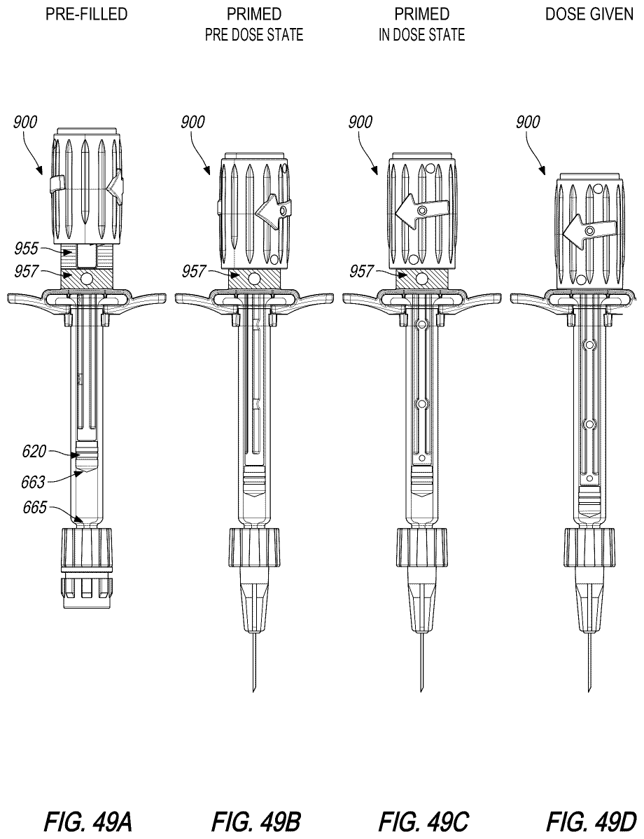

FIGS. 49A, 49B, 49C, and 49D are front views depicting a system 900 for injecting in various configurations according to some embodiments. The system 900 includes a first external indicia 955 of a first system transition between the transport configuration and the primed/injection ready configuration, and a second external indicia 957 of a second system transition between the primed/injection ready configuration and the injected configuration. The first indica is a purple color and the second indicia is a green color.

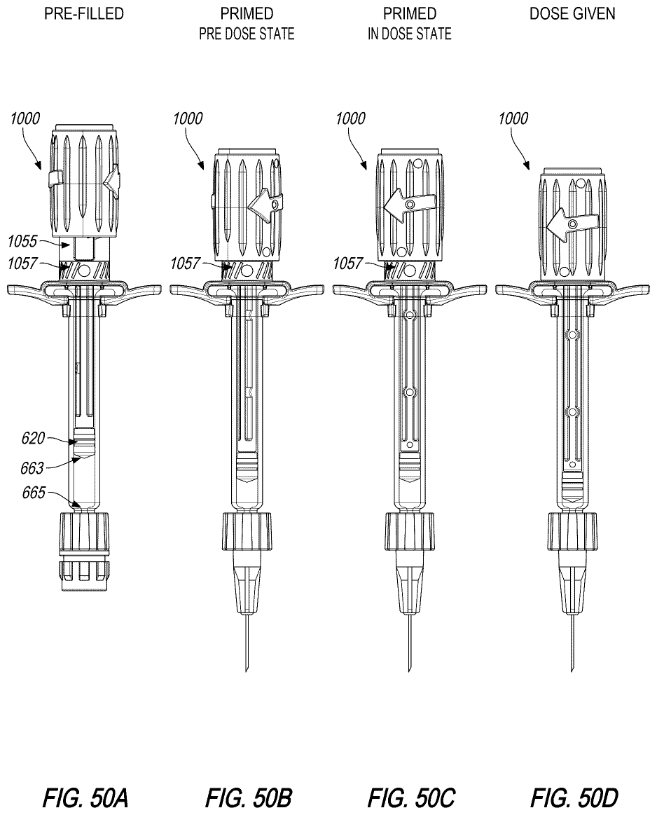

FIGS. 50A, 50B, 50C, and 50D are front views depicting a system 1000 for injecting in various configurations according to some embodiments. The system 1000 includes a first external indicia 1055 of a first system transition between the transport configuration and the primed/injection ready configuration, and a second external indicia 1057 of a second system transition between the primed/injection ready configuration and the injected configuration. The first indica is a blank pattern and the second indicia is a stippled pattern.

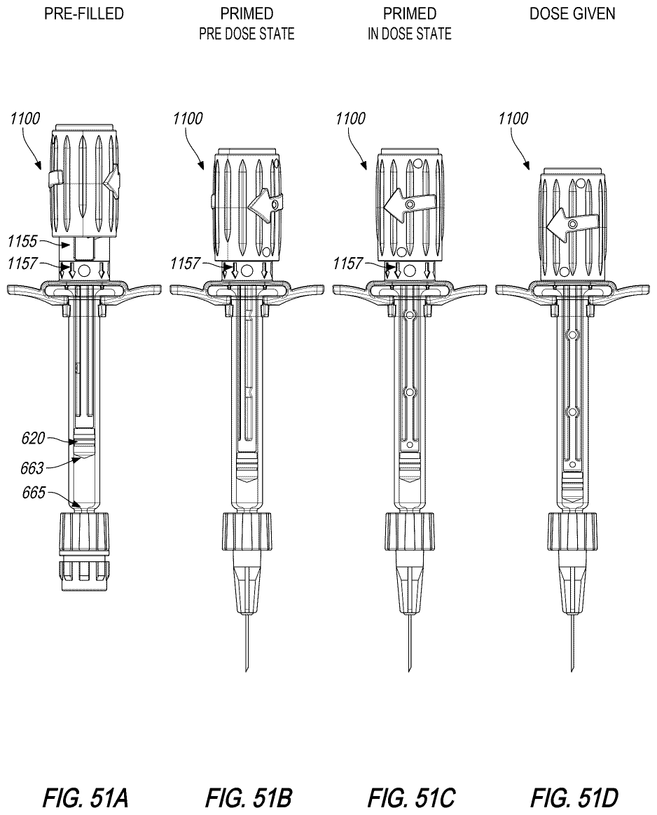

FIGS. 51A, 51B, 51C, and 51D are front views depicting a system 1100 for injecting in various configurations according to some embodiments. The system 1000 includes a first external indicia 1155 of a first system transition between the transport configuration and the primed/injection ready configuration, and a second external indicia 1157 of a second system transition between the primed/injection ready configuration and the injected configuration. The first indica is a blank pattern and the second indicia is a pattern with arrows.

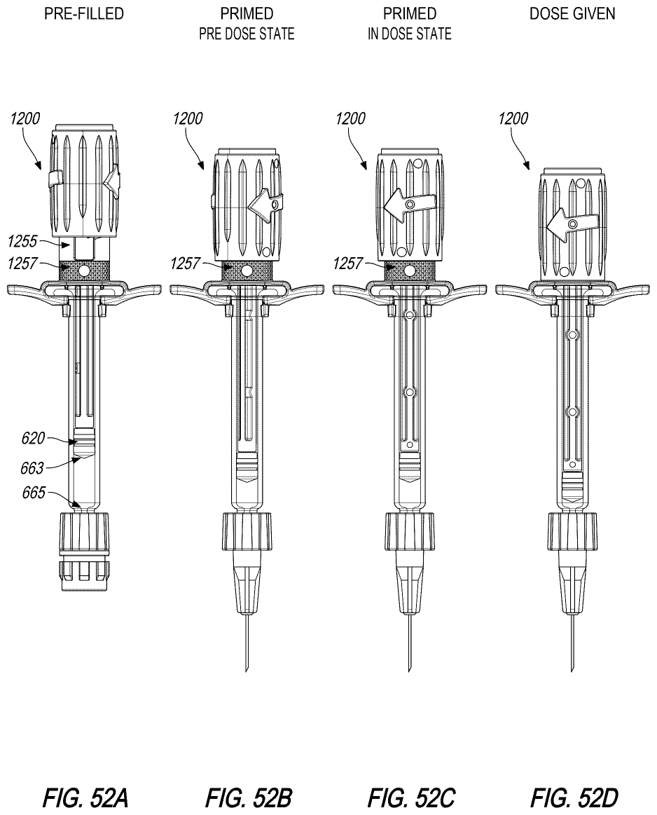

FIGS. 52A, 52B, 52C, and 52D are front views depicting a system 1200 for injecting in various configurations according to some embodiments. The system 1000 includes a first external indicia 1255 of a first system transition between the transport configuration and the primed/injection ready configuration, and a second external indicia 1257 of a second system transition between the primed/injection ready configuration and the injected configuration. The first indica is a smooth texture and the second indicia is a rough texture.

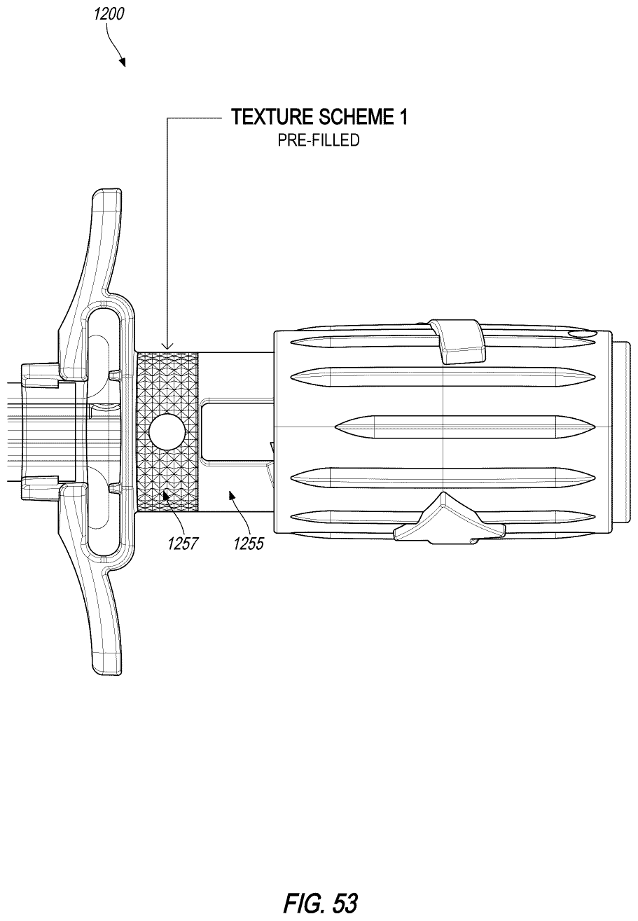

FIG. 53 is a detailed front view depicting a system 1200 for injecting according to some embodiments. The system 1000 includes a first external indicia 1255 of a first system transition between the transport configuration and the primed/injection ready configuration, and a second external indicia 1257 of a second system transition between the primed/injection ready configuration and the injected configuration. The first indica is a smooth texture and the second indicia is a rough texture.

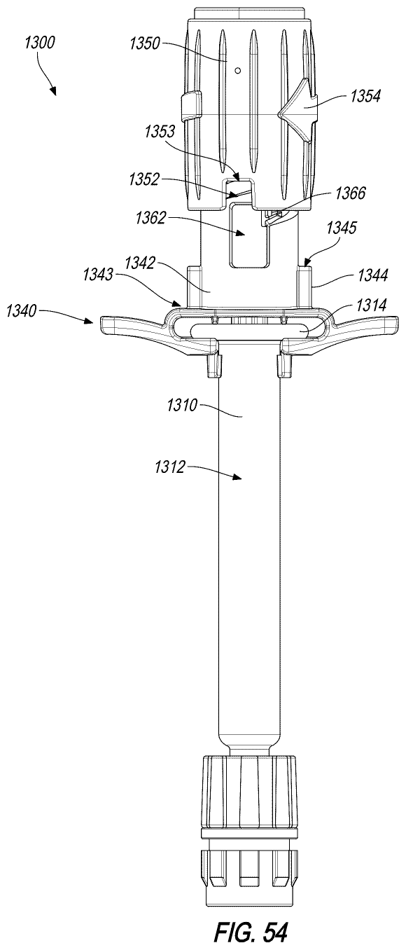

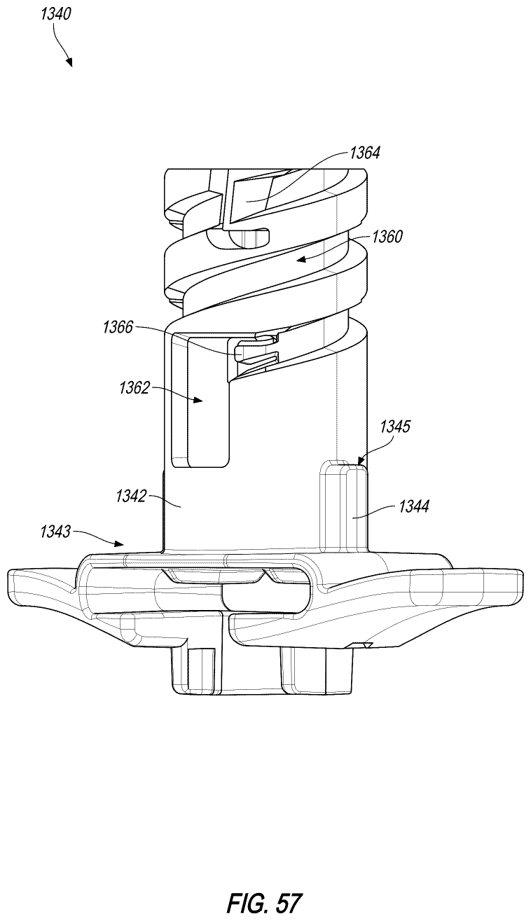

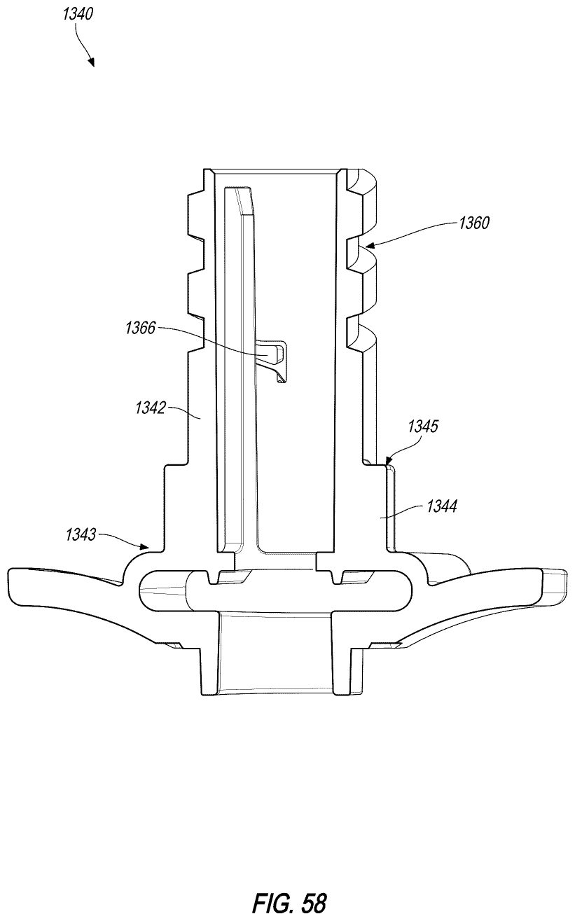

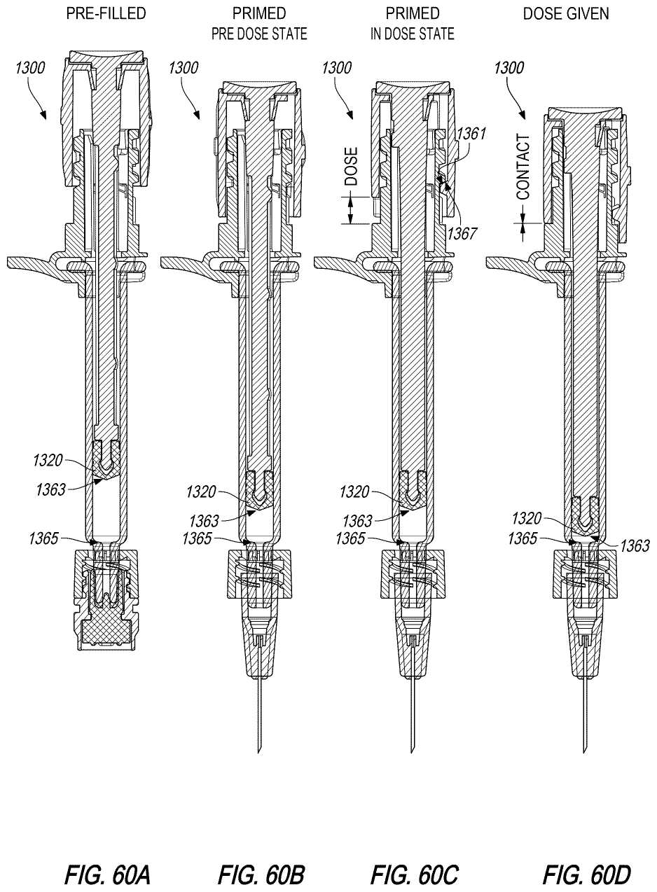

FIGS. 54 to 60D depict various aspects of a system/syringe assembly 1300 for injecting according to some embodiments. The system 1300 depicted in FIGS. 54 to 60D is similar to the system 700 depicted in FIGS. 30 to 48D, and function in a similar manner to perform an injection. The main difference between the two systems 700, 1300 is that the system 1300 depicted in FIGS. 54 to 60D has a pair of deflectable detents 1366 instead of a pair of radial bumps 766, which are found in the system 700 depicted in FIGS. 30 to 48D. As such, during the transition from the almost primed configuration (see e.g., FIG. 60B) to the primed/injection ready configuration (see e.g., FIG. 60C), the deflectable detents 1366 elastically deform with application of a deflecting force to allow respective inwardly extending members 1356 to move from respective external thread 1360 to respective distally extending external longitudinal slot 1362. The deflectable detents 1366 return to respective original positions after the deflecting force is removed therefrom in the primed/injection ready configuration (see e.g., FIG. 60C) and the injected configuration (see e.g., FIG. 60D). The deflectable detents 1366 returning to the respective original/neutral positions prevents the inwardly extending member 1356 from moving from the distally extending external longitudinal slot 1362 to the external thread 1360.

Like the system 700 depicted in FIGS. 30 to 48D, when the system 1300 depicted in FIGS. 54 to 60D is in the primed/injection ready configuration (see e.g., FIG. 60C), distal movement of a shroud 1350 relative to a finger flange 1340 is limited to a first longitudinal position by interference between a pair of inwardly extending members 1356 and corresponding external threads 1360 in a proximally extending member 1342 of the finger flange 1340. In the injected configuration (see e.g., FIG. 60D), a shroud distally facing surface 1351 is configured to interfere with proximally facing surfaces 1345 of the pair of flange elongate members 1344 to limit distal movement of the shroud 1350 relative to the finger flange 1340 to a second longitudinal position. In the injected configuration, the pair of inwardly extending members 1356 are not in contact with respective distal ends of the distally extending external longitudinal slots 1362 (see e.g., FIG. 60D).

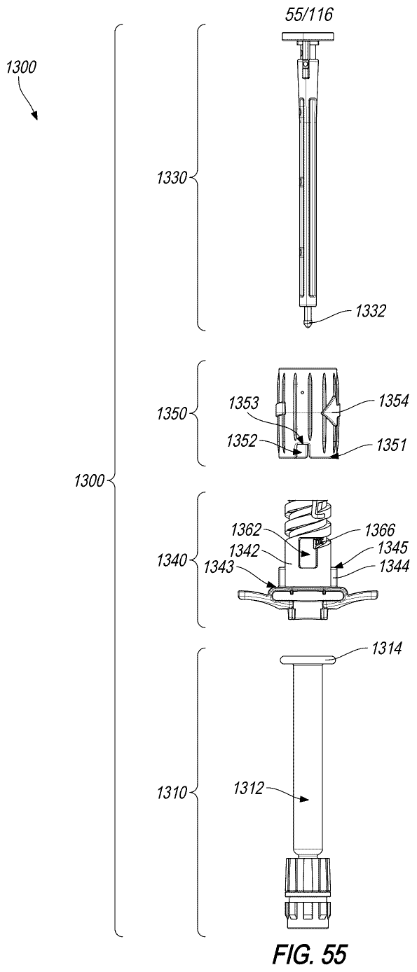

FIG. 54 is a front view depicting a system/syringe assembly 1300 for injecting according to some embodiments. FIG. 55 is an exploded view depicting the system/syringe assembly 1300 for injecting according to some embodiments. The system 1300 includes a syringe body 1310 having proximal and distal ends, a syringe interior 1312, and a syringe flange 1314 at the proximal end thereof. A stopper member 1320 is disposed in the syringe interior 1312 to form a fluid chamber therein. The syringe interior/fluid chamber 1312 contains an injectable fluid (not shown) and some air. A plunger member 1330 is partially disposed in the syringe interior 1312. The plunger member 1330 has a distal end 1332 disposed adjacent to the stopper member 1320. The system 1300 also includes a finger flange 1340 coupled to the syringe flange 1312. The system 1300 further includes a shroud 1350 coupled to the plunger member 1330 and movable relative to the finger flange 1340.

The plunger member 1330 is rotatably and translationally coupled to the shroud 1350. While the distal end 1332 of the plunger member 1330 of the system depicted in FIG. 55 has an interference type connector for a stopper member (not shown), in other embodiments the distal end of the plunger member may have a screw type connector to couple the plunger member to the stopper member or a flat end that only pushes stopper members distally without the ability to pull stopper members proximally.

The finger flange 1340 defines a proximally extending member 1342, which has a pair of external threads 1360 defined thereon. The external threads 1360 are double lead threads with about a 5 mm separation there between. The proximally extending member 1342 also defines a pair of distally extending external longitudinal slot 1362 at respective distal ends of the external threads 1360. The finger flange 1340 defines a flange proximally facing surface 1343 and a pair of flange elongate members 1344 proximally extending from the flange proximally facing surface 1343. While a pair of flange elongate members 1344 are shown, alternative embodiments may have a single flange elongate member, or 3 or more flange elongate members.

The shroud 1350 defines a shroud distally facing surface 1351 at a distal end thereof configured to interfere with a proximally facing surface 1345 of the flange elongate member 1344 to limit distal movement of the shroud 1350 to a first longitudinal position on the finger flange 1340 when the shroud 1350 is in a first rotational state (see e.g., FIG. 60C) relative to the finger flange 1340. The shroud 1350 also defines a slot 1352 extending proximally from the distally facing surface 1351 and configured to receive the flange elongate member 1344 to allow distal movement of the shroud 1340 distally beyond the first longitudinal position on the finger flange 1350 when the shroud 1340 is in a second rotational state (see e.g., FIG. 60D) relative to the finger flange 1340. The slot 1352 has a distally facing surface 1353. While two slots 1352 are shown, alternative embodiments may have one slot or 3 or more slots. The shroud 1350 also defines an external indicia of rotation direction 1354. The shroud 1350 has a knurled external surface to facilitate manual rotation thereof.

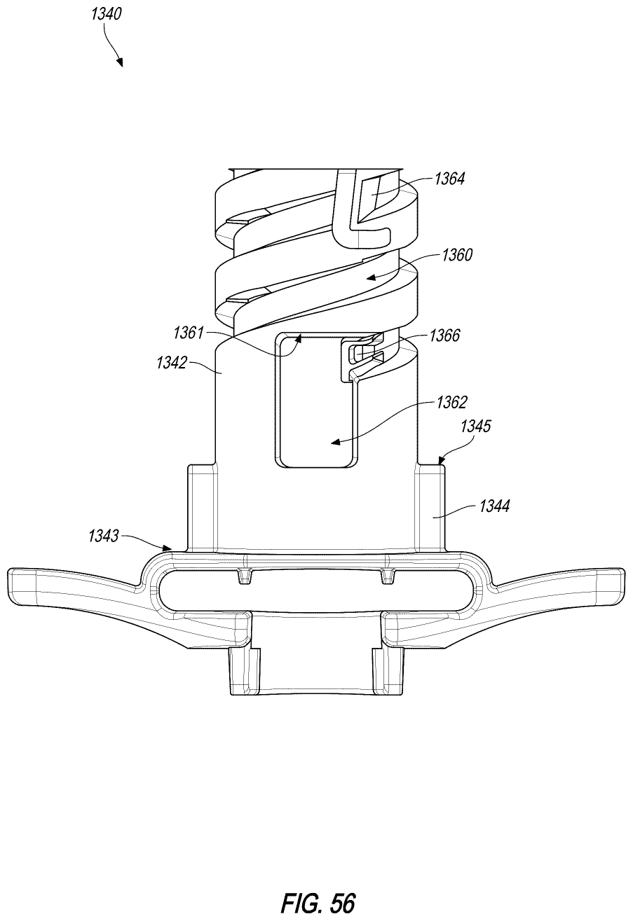

FIGS. 56, 57, and 58 are front, perspective, and longitudinal cross-sectional views depicting a finger flange 1340 for use with systems for injecting, such as the system 1300 depicted in FIGS. 54 and 55, according to some embodiments. The finger flange 1340 defines a flange proximally facing surface 1343 and a proximally extending member 1342, which has a pair of external threads 1360 defined thereon. The external threads 1360 are double lead threads with about a 5 mm separation there between. The proximally extending member 1342 also defines a pair of flange elongate members 1344, each having a flange elongate member proximally facing surface 1345. The proximally extending member 1342 further defines a pair of distally extending external longitudinal slot 1362 at respective distal ends of the external threads 1360. Moreover, the proximally extending member 1342 further defines a pair of proximal bumps 1364 adjacent respective proximal ends of the external threads 1360 and a pair of deflectable detents 1366 adjacent respective distal ends of the external threads 1360.

While the threaded connection illustrated in the embodiment herein is a double-lead or 2-start thread, the threads may be a single lead or have 3 or more leads. The thread lead or pitch or distance traveled in one thread revolution may be between 2 and 10 mm. In a preferred embodiment, the thread lead is 8.57 mm. The threaded connection may be configured with a thread lead or pitch that makes the thread self-locking, where the coefficient of friction between the finger flange and the shroud is greater than or equal to the tangent of the lead angle of the thread. Where the lead angle of the thread is the inverse tangent of the lead divided by TT times the thread diameter. In a self-locking thread, a proximally directed force that the stopper member exerts on the plunger member and the intercoupled shroud during use, manufacturing, or shipment will not rotate and/or translate the threaded shroud and intercoupled plunger member in a proximal direction. During terminal sterilization of a filled and closed syringe body, with the finger flange attached, pressure may build up in the medicine chamber due to the temperature increase. This pressure may cause a proximally directed force to be applied to the stopper member, urging the stopper to move proximally. It is desired not to allow the stopper to move more than the distance between the sealing ribs of the stopper member (see e.g., FIG. 5A) to prevent the exposure of potentially non-sterile surfaces of the stopper member and/or syringe body to the injectable fluid. The self-locking threaded connection between the finger flange and the shroud, and the intercoupled plunger member and stopper member prevents the movement of the stopper member proximally until the temperature of the prefilled syringe body is reduced and the pressure in the syringe interior is reduced. Additionally, during air shipment the filled, stoppered, and assembled syringe bodies may experience a drop in atmospheric pressure external to the syringe bodies. With a drop in external pressure, the injectable fluid and any entrained air bubbles in the syringe body may expand and apply a proximally directed force on the stopper member. It is desired not to allow the stopper member to move more than the distance between the sealing ribs of the stopper member to prevent the exposure of potentially non-sterile surfaces of the stopper member and/or syringe body to the injectable fluid. The self-locking threaded connection between the finger flange and shroud and the intercoupled plunger member and stopper member prevents the movement of the stopper member proximally until the external atmospheric pressure is returned to an amount similar to the pressure in the syringe interior.

The deflectable detents 1366 are configured to cooperate with respective inwardly extending members 1356 to control movement of the inwardly extending members 1356 between the external thread 1360 and the distally extending external longitudinal slot 1362. The deflectable detents 1366 separate the distal ends of the external threads 1360 from the proximal ends of the longitudinal slots 1362. During the transition from the almost primed configuration (see e.g., FIG. 60B) to the primed/injection ready configuration (see e.g., FIG. 60C), the deflectable detents 1366 elastically deform with application of a deflecting force (e.g., by respective inwardly extending members 1356) in a radial direction to allow the inwardly extending members 1356 to move from respective external thread 1360 to respective distally extending external longitudinal slot 1362. The deflectable detents 1366 return to respective original positions after the deflecting force is removed therefrom in the primed/injection ready configuration (see e.g., FIG. 60C) and the injected configuration (see e.g., FIG. 60D). The deflectable detents 1366 returning to the respective original/neutral positions prevents the inwardly extending member 1356 from moving from the distally extending external longitudinal slot 1362 to the external thread 1360. The deflectable detents 1366 may be biased to return to the respective original/neutral positions. The release/return of the deflectable detents 1366 to the respective original/neutral position may generate audible and/or tactile cues or feedback to the user to indicate that the shroud 1350 has rotated into the dose start position and the injection system 1300 is in the primed/injection ready configuration and ready for the user to inject fluid into the patient. The deflectable detents 1366 may also be self-energized (e.g., in response to a force applied by respective inwardly extending members 1356) to return to the respective original/neutral positions. The deflectable detents 1366 may be configured to cooperate with respective inwardly extending members 1356 to generate a tactile and/or audible indicator when the system 1300 transitions from the almost primed configuration (see e.g., FIG. 60B) to the primed/injection ready configuration (see e.g., FIG. 60C).

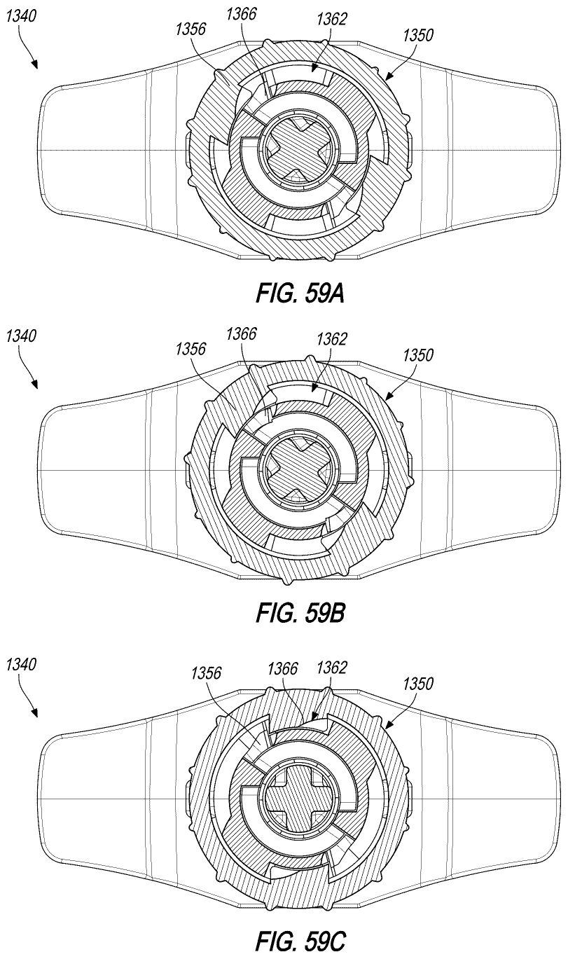

FIGS. 59A, 59B, and 59C are transverse cross-sectional views depicting a system for injecting, such as the system 1300 depicted in FIGS. 54 and 55, in various configurations according to some embodiments. In FIG. 59A, the system 1300 is in the almost primed/injection ready configuration depicted in FIG. 60B, with the pair of inwardly extending members 1356 of the shroud 1350 adjacent to, but not past, respective deflectable detents 1366 of the flinger flange 1340. In FIG. 59B, the deflectable detents 1366 of the flinger flange 1340 have been deflected radially inward by a deflecting force generated by the inwardly extending members 1356 rotating over a ramp on the deflectable detent 1366. The deflectable detents 1366 deflecting radially inward allows respective inwardly extending members 1356 to move from respective external thread 1360 to respective distally extending external longitudinal slot 1362. In FIG. 59C, the system 1300 has been transformed, by rotation of the shroud 1350 and plunger member 1330 relative to the finger flange 1340, to the primed/injection ready configuration depicted in FIG. 60C. In FIG. 59C, the deflectable detents 1366 return to respective original positions after the deflecting force is removed therefrom in the primed/injection ready configuration (see e.g., FIG. 60C) and the injected configuration (see e.g., FIG. 60D). The deflectable detents 1366 returning to the respective original/neutral positions prevents the inwardly extending member 1356 from moving from the distally extending external longitudinal slot 1362 to the external thread 1360. The deflectable detents 1366 may be biased to return to the respective original/neutral positions. The deflectable detents 1366 may also be self-energized (e.g., in response to a force applied by counter-rotation of respective inwardly extending members 1356) to return to the respective original/neutral positions. In the primed/injection ready configuration, proximally facing surfaces 1367 of the inwardly extending members 1356 of the shroud 1350 and the circumferential surface 1361 of the finger flange external thread 1360 are in contact to define the dose start position of the shroud 1350 and the intercoupled plunger member 1330 and stopper member 1320 (see e.g., FIG. 60C).

During manufacturing, shipping, and storage prior to use the stopper 1320 may experience additional initial break loose friction also known as stiction with the syringe body 1310. This stiction results in an increased amount of axial force required to break the stopper 1320 free from the syringe body 1310 during priming. This stiction may also be increased by the desire to limit silicone oil lubrication on the inside of the syringe body 1310 for ophthalmological and or intravitreal injections. The priming mechanism described herein provides a mechanical advantage to aide in breaking loose the stopper during priming. In particular, the shroud projection 1356 is configured to engage the helical grooves/threads 1360 of the finger flange 1340 providing a threaded connection between the shroud 1350 and the finger flange 1340. This threaded connection provides a mechanical advantage to the user allowing the stopper 1320 to be freed from the initial stiction with a break loose force applied tangential to the outside diameter of the shroud 1350 by the user to break loose the stopper 1320 from the syringe body 1310. As the plunger member 1330 is advanced distally relative to the finger flange 1340 the system 1300 is positioned in an upward orientation to allow an advancing stopper member (not shown) to eject air from a syringe interior (not shown).

FIGS. 60A, 60B, 60C, and 60D are longitudinal cross-sectional views depicting a system for injecting, such as the system 1300 depicted in FIGS. 54 and 55, in various configurations according to some embodiments. In FIG. 60A, the system 1300 is in the transport configuration depicted in FIG. 54. In FIG. 60B, the system 1300 is in the almost primed/injection ready configuration depicted in FIG. 59A. In FIG. 60C, the system 1300 is in the primed/injection ready configuration. In FIG. 60D, the system 1300 is in the injected configuration depicted in FIG. 59C. The plunger rod length is configured such that when contact is made between the distally facing surfaces 1353 of the shroud 1350 and the proximally facing surfaces 1345 of the finger flange 1340, the distal surface 1363 of the stopper 1320 is spaced from the syringe barrel bottom surface 1365. This spacing allows the volume of the delivered dose to be independent of the manufacturing tolerance of the syringe barrel length, allowing enhanced precision and accuracy of the delivered dose volume.

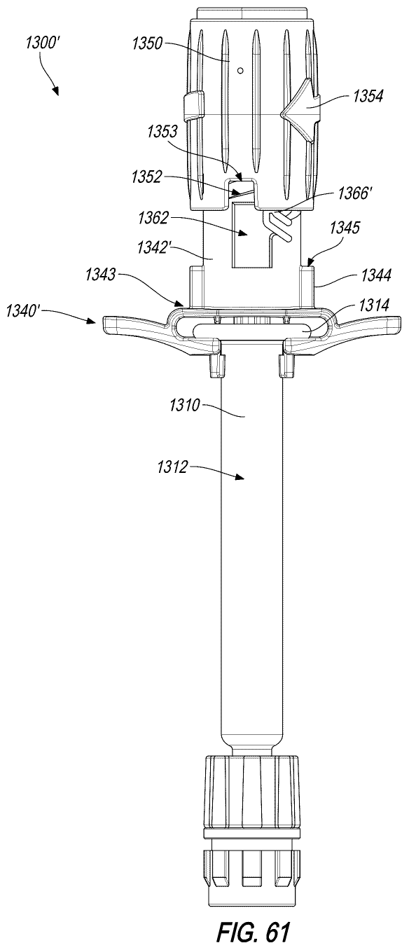

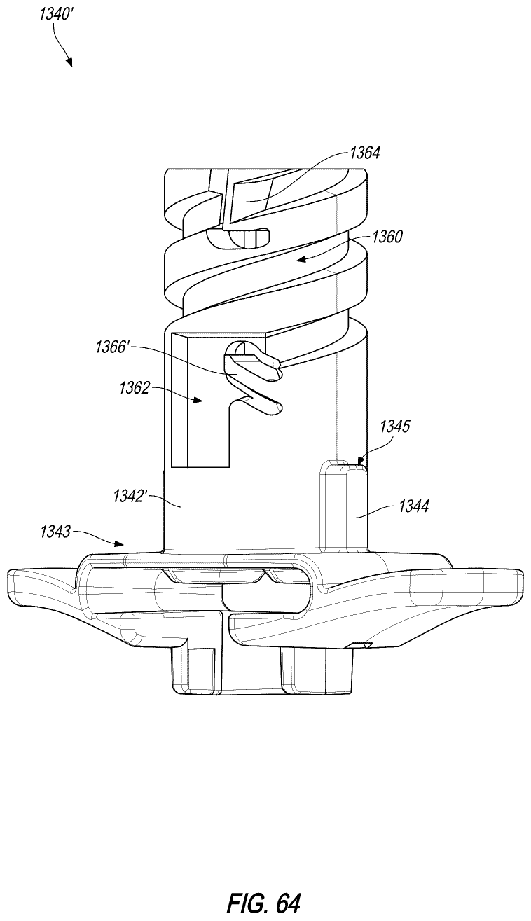

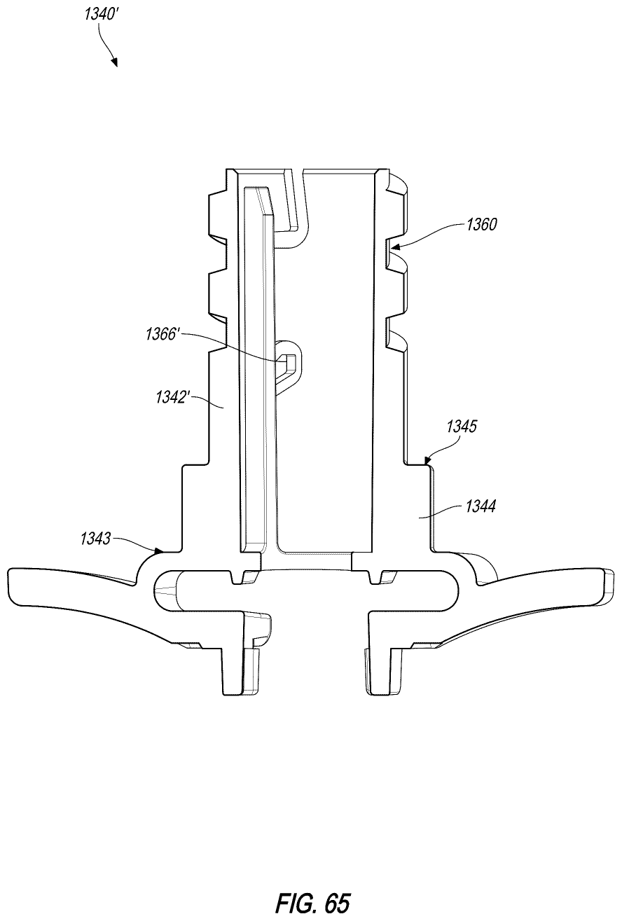

FIGS. 61 to 69D depict various aspects of a system/syringe assembly 1300′ for injecting according to some embodiments. The system 1300′ depicted in FIGS. 61 to 69D is very similar to the system 1300 depicted in FIGS. 54 to 60D, and function in a similar manner to perform an injection. The main difference between the two systems 1300, 1300′ is that the system 1300′ depicted in FIGS. 61 to 69D has a pair of deflectable detents 1366′ that deflect longitudinally/axially instead radially like the pair of deflectable detents 1366 in the system 1300 depicted in FIGS. 54 to 60D.

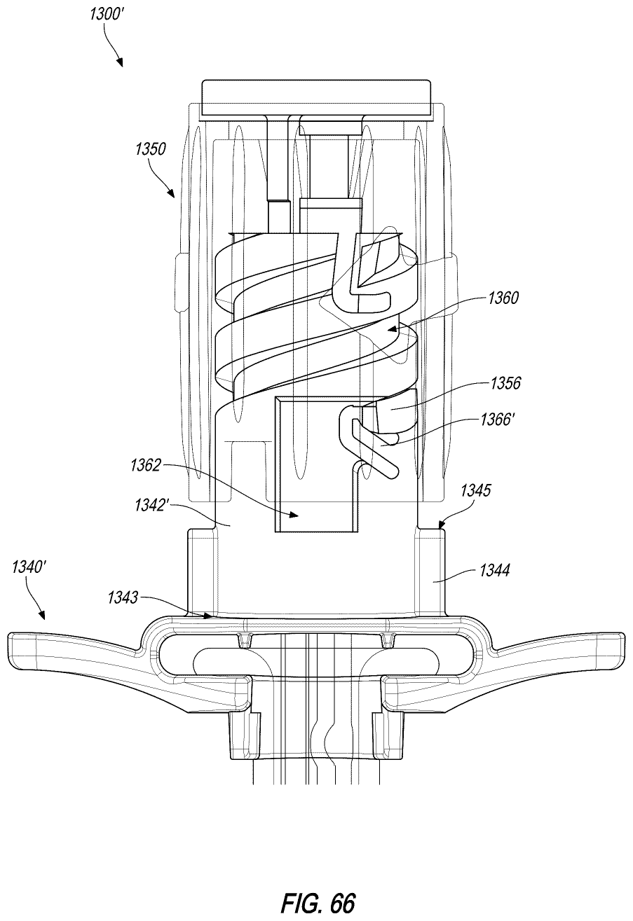

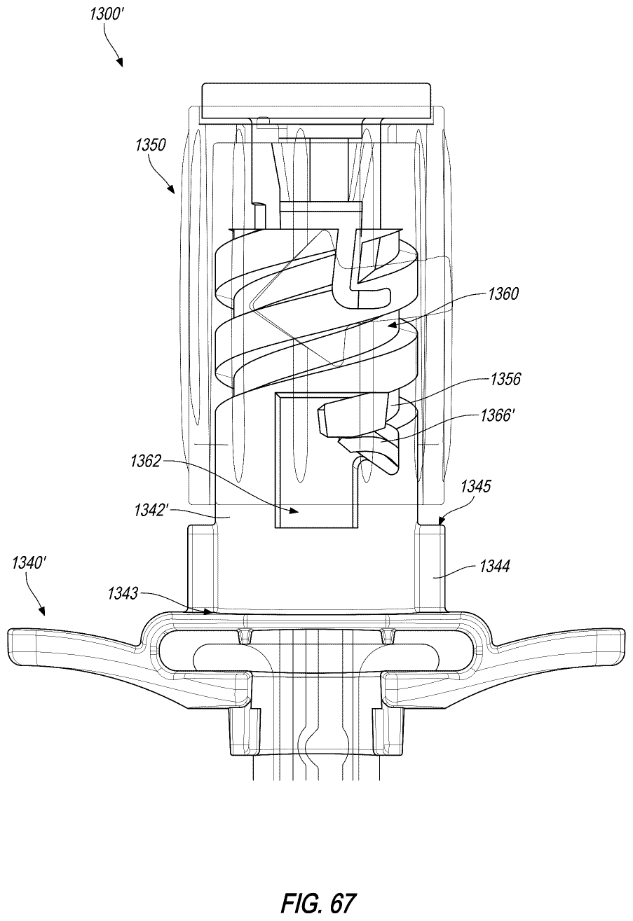

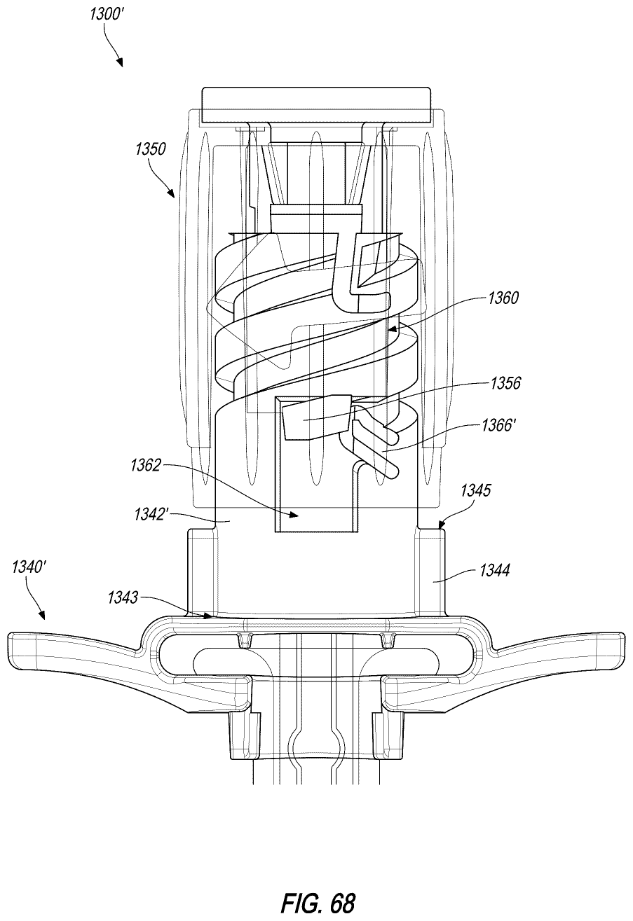

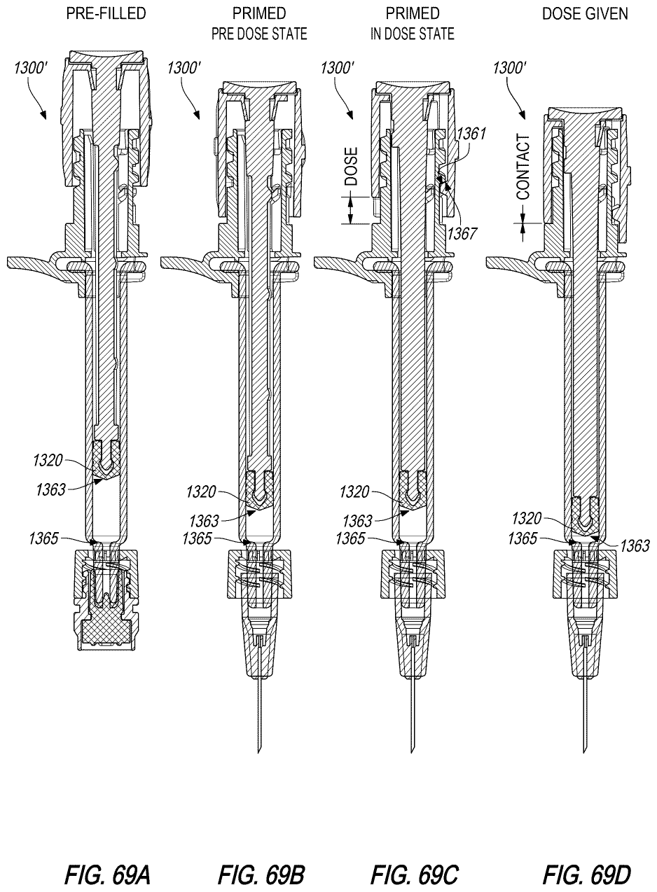

Like the system 1300 depicted in FIGS. 54 to 60D, during the transition from the almost primed configuration (see e.g., FIGS. 66 and 69B) to the primed/injection ready configuration (see e.g., FIGS. 68 and 69C), the deflectable detents 1366′ elastically deform (see e.g., FIG. 67) with application of a deflecting force to allow respective inwardly extending members 1356 to move from respective external thread 1360 to respective distally extending external longitudinal slot 1362. The deflectable detents 1366′ return to respective original positions after the deflecting force is removed therefrom in the primed/injection ready configuration (see e.g., FIGS. 68 and 69C) and the injected configuration (see e.g., FIG. 69D). The deflectable detents 1366′ returning to the respective original/neutral positions prevents the inwardly extending member 1356 from moving from the distally extending external longitudinal slot 1362 to the external thread 1360. The release/return of the deflectable detents 1366′ to the respective original/neutral position may generate audible and/or tactile cues or feedback to the user to indicate that the shroud 1350 has rotated into the dose start position and the injection system 1300 is in the primed/injection ready configuration and ready for the user to inject fluid into the patient.

Like the system 1300 depicted in FIGS. 54-60D, when the system 1300′ depicted in 61 to 69D is in the primed/injection ready configuration (see e.g., FIGS. 68 and 69C), distal movement of a shroud 1350 relative to a finger flange 1340′ is limited to a first longitudinal position by interference between a pair of inwardly extending members 1356 and corresponding external threads 1360 in a proximally extending member 1342′ of the finger flange 1340′. In the injected configuration (see e.g., FIG. 69D), the distally facing surface 1353 of the slot 1362 is configured to interfere with the proximally facing surfaces 1345 of the pair of flange elongate members 1344. In the injected configuration, the pair of inwardly extending members 1356 are not in contact with respective distal ends of the distally extending external longitudinal slots 1362 (see e.g., FIG. 69D).

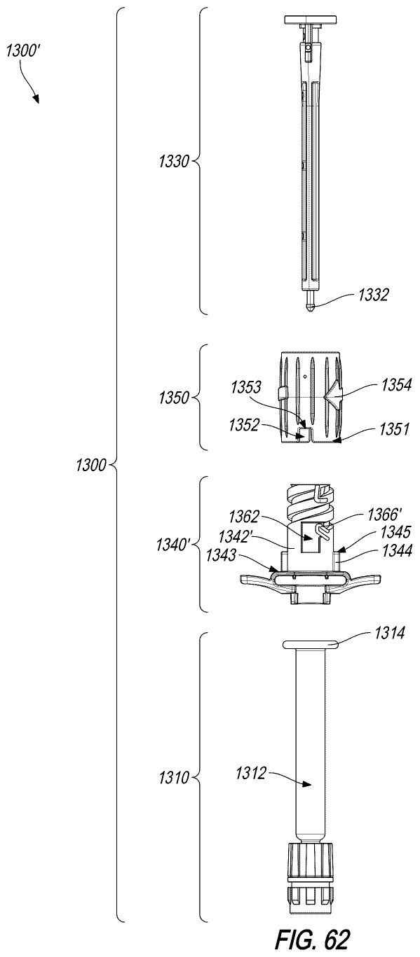

FIG. 61 is a front view depicting a system/syringe assembly 1300′ for injecting according to some embodiments. FIG. 62 is an exploded view depicting the system/syringe assembly 1300′ for injecting according to some embodiments. The system 1300′ includes a syringe body 1310 having proximal and distal ends, a syringe interior 1312, and a syringe flange 1314 at the proximal end thereof. A stopper member 1320 is disposed in the syringe interior 1312 to form a fluid chamber therein. The syringe interior/fluid chamber 1312 contains an injectable fluid (not shown) and some air. A plunger member 1330 is partially disposed in the syringe interior 1312. The plunger member 1330 has a distal end 1332 disposed adjacent to the stopper member 1320. The system 1300′ also includes a finger flange 1340′ coupled to the syringe flange 1312. The system 1300′ further includes a shroud 1350 coupled to the plunger member 1330 and movable relative to the finger flange 1340′.

The plunger member 1330 is rotatably and translationally coupled to the shroud 1350. While the distal end 1332 of the plunger member 1330 of the system depicted in FIG. 62 has an interference type connector for a stopper member (not shown), in other embodiments the distal end of the plunger member may have a screw type connector to couple the plunger member to the stopper member or a flat end that only pushes stopper members distally without the ability to pull stopper members proximally.

The finger flange 1340′ defines a proximally extending member 1342′, which has a pair of external threads 1360 defined thereon. The external threads 1360 are double lead threads with about a 5 mm separation there between. The proximally extending member 1342′ also defines a pair of distally extending external longitudinal slot 1362 at respective distal ends of the external threads 1360. The finger flange 1340′ defines a flange proximally facing surface 1343.

The shroud 1350 defines a shroud distally facing surface 1351 at a distal end thereof configured to interfere with proximally facing surfaces 1345 of the pair of flange elongate members 1344 to limit distal movement of the shroud 1350 to a second longitudinal position on the finger flange 1340′ when the shroud 1350 is in a first rotational state (see e.g., FIG. 60D) relative to the finger flange 1340′. The shroud 1350 also defines an external indicia of rotation direction 1354. The shroud 1350 has a knurled external surface to facilitate manual rotation thereof.

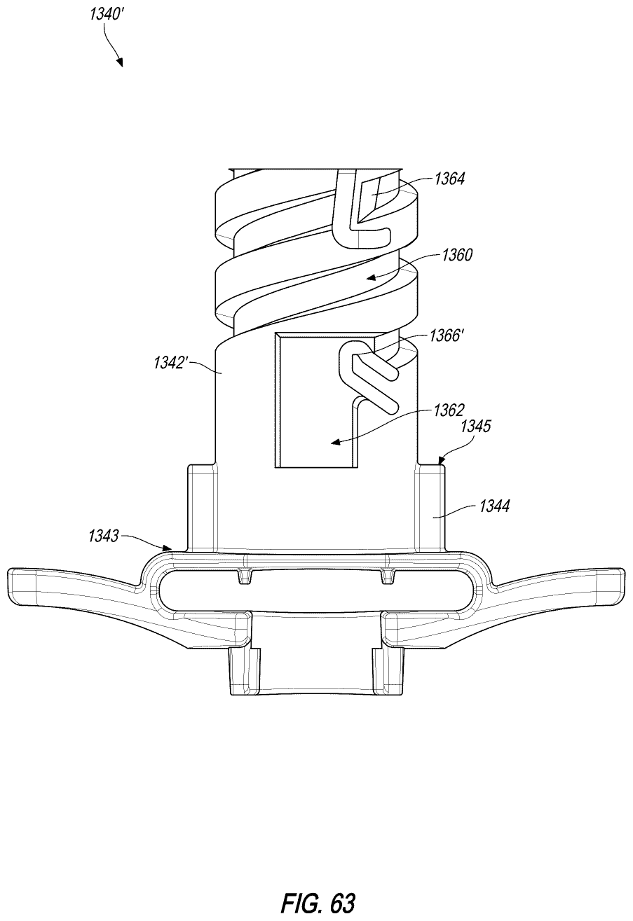

FIGS. 63, 64, and 65 are front, perspective, and longitudinal cross-sectional views depicting a finger flange 1340′ for use with systems for injecting, such as the system 1300′ depicted in FIGS. 61 and 62, according to some embodiments. The finger flange 1340′ defines a flange proximally facing surface 1343 and a proximally extending member 1342′, which has a pair of external threads 1360 defined thereon. The external threads 1360 are double lead threads with about a 5 mm separation there between. The proximally extending member 1342′ also defines a pair of distally extending external longitudinal slot 1362 at respective distal ends of the external threads 1360. The proximally extending member 1342′ further defines a pair of proximal bumps 1364 adjacent respective proximal ends of the external threads 1360 and a pair of deflectable detents 1366′ adjacent respective distal ends of the external threads 1360.

While the threaded connection illustrated in the embodiment herein is a double-lead or 2-start thread, the threads may be a single lead or have 3 or more leads. The thread lead or pitch or distance traveled in one thread revolution may be between 2 and 10 mm. In a preferred embodiment, the thread lead is 8.57 mm. The threaded connection may be configured with a thread lead or pitch that makes the thread self-locking, where the coefficient of friction between the finger flange and the shroud is greater than or equal to the tangent of the lead angle of the thread. Where the lead angle of the thread is the inverse tangent of the lead divided by TT times the thread diameter. In a self-locking thread, a proximally directed force that the stopper member exerts on the plunger member and the intercoupled shroud during use, manufacturing, or shipment will not rotate and/or translate the threaded shroud and intercoupled plunger member in a proximal direction. During terminal sterilization of a filled and closed syringe body, with the finger flange attached, pressure may build up in the medicine chamber due to the temperature increase. This pressure may cause a proximally directed force to be applied to the stopper member, urging the stopper to move proximally. It is desired not to allow the stopper to move more than the distance between the sealing ribs of the stopper member (see e.g., FIG. 5A) to prevent the exposure of potentially non-sterile surfaces of the stopper member and/or syringe body to the injectable fluid. The self-locking threaded connection between the finger flange and the shroud, and the intercoupled plunger member and stopper member prevents the movement of the stopper member proximally until the temperature of the prefilled syringe body is reduced and the pressure in the syringe interior is reduced. Additionally, during air shipment the filled, stoppered, and assembled syringe bodies may experience a drop in atmospheric pressure external to the syringe bodies. With a drop in external pressure, the injectable fluid and any entrained air bubbles in the syringe body may expand and apply a proximally directed force on the stopper member. It is desired not to allow the stopper member to move more than the distance between the sealing ribs of the stopper member to prevent the exposure of potentially non-sterile surfaces of the stopper member and/or syringe body to the injectable fluid. The self-locking threaded connection between the finger flange and shroud and the intercoupled plunger member and stopper member prevents the movement of the stopper member proximally until the external atmospheric pressure is returned to an amount similar to the pressure in the syringe interior.

The deflectable detents 1366′ are configured to cooperate with respective inwardly extending members 1356 to control movement of the inwardly extending members 1356 between the external thread 1360 and the distally extending external longitudinal slot 1362. The deflectable detents 1366′ separate the distal ends of the external threads 1360 from the proximal ends of the longitudinal slots 1362. During the transition from the almost primed configuration (see e.g., FIGS. 66 and 69B) to the primed/injection ready configuration (see e.g., FIGS. 68 and 69C), the deflectable detents 1366′ elastically deform with application of a deflecting force (e.g., by respective inwardly extending members 1356; see e.g., FIG. 67) in a longitudinal/axial direction to allow the inwardly extending members 1356 to move from respective external thread 1360 to respective distally extending external longitudinal slot 1362. The deflectable detents 1366′ return to respective original positions after the deflecting force is removed therefrom in the primed/injection ready configuration (see e.g., FIGS. 68 and 69C) and the injected configuration (see e.g., FIG. 69D). The deflectable detents 1366′ returning to the respective original/neutral positions prevents the inwardly extending member 1356 from moving from the distally extending external longitudinal slot 1362 to the external thread 1360. The deflectable detents 1366′ may be biased to return to the respective original/neutral positions. The deflectable detents 1366′ may also be self-energized (e.g., in response to a force applied by respective inwardly extending members 1356) to return to the respective original/neutral positions. The deflectable detents 1366′ may be configured to cooperate with respective inwardly extending members 1356 to generate a tactile and/or audible indicator when the system 1300′ transitions from the almost primed configuration (see e.g., FIGS. 66 and 69B) to the primed/injection ready configuration (see e.g., FIGS. 68 and 69C).

FIGS. 66, 67, and 68 are front views depicting a system for injecting, such as the system 1300′ depicted in FIGS. 61 and 62, with the shroud 1350 shown in shadow for clarity, in various configurations according to some embodiments. In FIG. 66, the system 1300′ is in the almost primed/injection ready configuration with the pair of inwardly extending members 1356 of the shroud 1350 adjacent to, but not past, respective deflectable detents 1366′ of the flinger flange 1340′, as shown in FIG. 69B. In FIG. 67, the deflectable detents 1366′ of the flinger flange 1340′ have been deflected longitudinally/axially by a deflecting force generated by the inwardly extending members 1356 rotating over a ramp on the deflectable detent 1366′. The deflectable detents 1366′ deflecting longitudinally/axially allows respective inwardly extending members 1356 to move from respective external thread 1360 to respective distally extending external longitudinal slot 1362. In FIG. 68, the system 1300′ has been transformed, by rotation of the shroud 1350 and plunger member 1330 relative to the finger flange 1340′, to the primed/injection ready configuration shown in FIG. 69C. In FIG. 68, the deflectable detents 1366′ return to respective original positions after the deflecting force is removed therefrom in the primed/injection ready configuration (see e.g., FIGS. 68 and 69C) and the injected configuration (see e.g., FIG. 69D). The deflectable detents 1366′ returning to the respective original/neutral positions prevents the inwardly extending member 1356 from moving from the distally extending external longitudinal slot 1362 to the external thread 1360. The deflectable detents 1366′ may be biased to return to the respective original/neutral positions. The deflectable detents 1366′ may also be self-energized (e.g., in response to a force applied by counter-rotation of respective inwardly extending members 1356) to return to the respective original/neutral positions.

During manufacturing, shipping, and storage prior to use the stopper 1320 may experience additional initial break loose friction also known as stiction with the syringe body 1310. This stiction results in an increased amount of axial force required to break the stopper 1320 free from the syringe body 1310 during priming. This stiction may also be increased by the desire to limit silicone oil lubrication on the inside of the syringe body 1310 for ophthalmological and or intravitreal injections. The priming mechanism described herein provides a mechanical advantage to aide in breaking loose the stopper during priming. In particular, the shroud projection 1356 is configured to engage the helical grooves/threads 1360 of the finger flange 1340′ providing a threaded connection between the shroud 1350 and the finger flange 1340′. This threaded connection provides a mechanical advantage to the user allowing the stopper 1320 to be freed from the initial stiction with a break loose force applied tangential to the outside diameter of the shroud 1350 by the user to break loose the stopper 1320 from the syringe body 1310. As the plunger member 1330 is advanced distally relative to the finger flange 1340′ the system 1300′ is positioned in an upward orientation to allow an advancing stopper member (not shown) to eject air from a syringe interior (not shown).

FIGS. 69A, 69B, 69C, and 69D are longitudinal cross-sectional views depicting a system for injecting, such as the system 1300′ depicted in FIGS. 61 and 62, in various configurations according to some embodiments. In FIG. 69A, the system 1300′ is in the transport configuration. In FIG. 69B, the system 1300′ is in the almost primed/injection ready configuration shown in FIG. 67. In FIG. 69C, the system 1300′ is in the primed/injection ready configuration shown in FIG. 68. In FIG. 69D, the system 1300′ is in the injected configuration. The plunger rod length is configured such that when contact is made between the distally facing surfaces 1351 of the shroud 1350 and the proximally facing surfaces 1345 of the pair of flange elongate members 1344, the distal surface 1363 of the stopper 1320 is spaced from the syringe barrel bottom surface 1365. This spacing allows the volume of the delivered dose to be independent of the manufacturing tolerance of the syringe barrel length, allowing enhanced precision and accuracy of the delivered dose volume. In the primed/injection ready configuration, proximally facing surfaces 1367 of the inwardly extending members 1356 of the shroud 1350 and the circumferential surface 1361 of the finger flange external thread 1360 are in contact to define the dose start position of the shroud 1350 and the intercoupled plunger member 1330 and stopper member 1320 (see e.g., FIG. 69C).

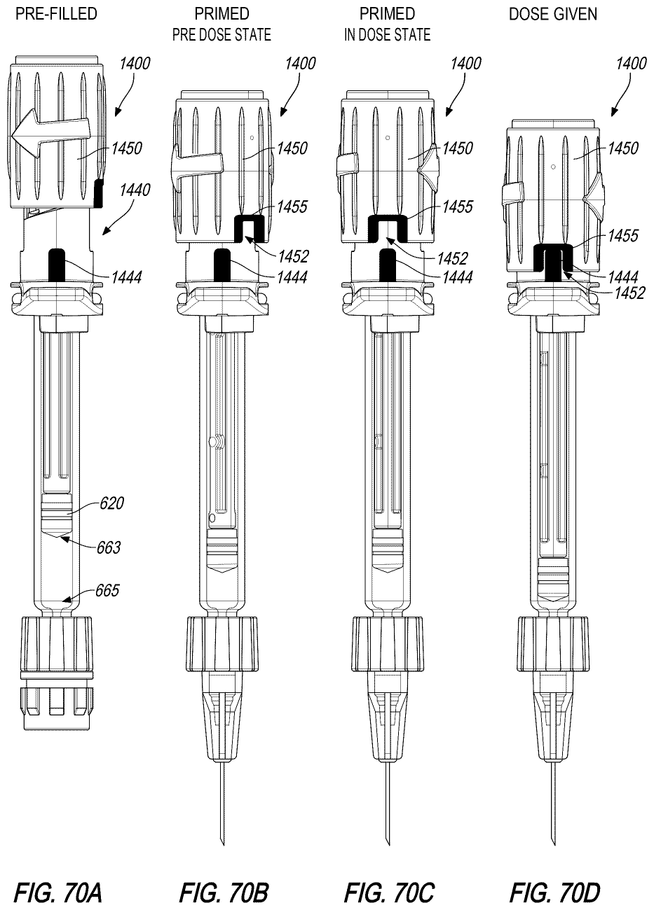

FIGS. 70A, 70B, 70C, and 70D are side views depicting a system 1400 for injecting in various configurations according to some embodiments. The system 1400 includes a pair of flange elongate members 1444 on the finger flange 1440 and a pair of slots 1452 formed in the shroud 1450. The slots 1452 include respective borders 1455. The flange elongate members 1444 and the borders 1455 of the slots 1452 have the same color, which is unique in the system 1400. The color of the flange elongate members 1444 and the borders 1455 is black compared to the rest of the system 1400, which is grey. The flange elongate members 1444 and the borders 1455 share the same color to indicate to a user the amount of rotation to transform the system 1400 from the transport configuration to the primed/injection ready configuration.

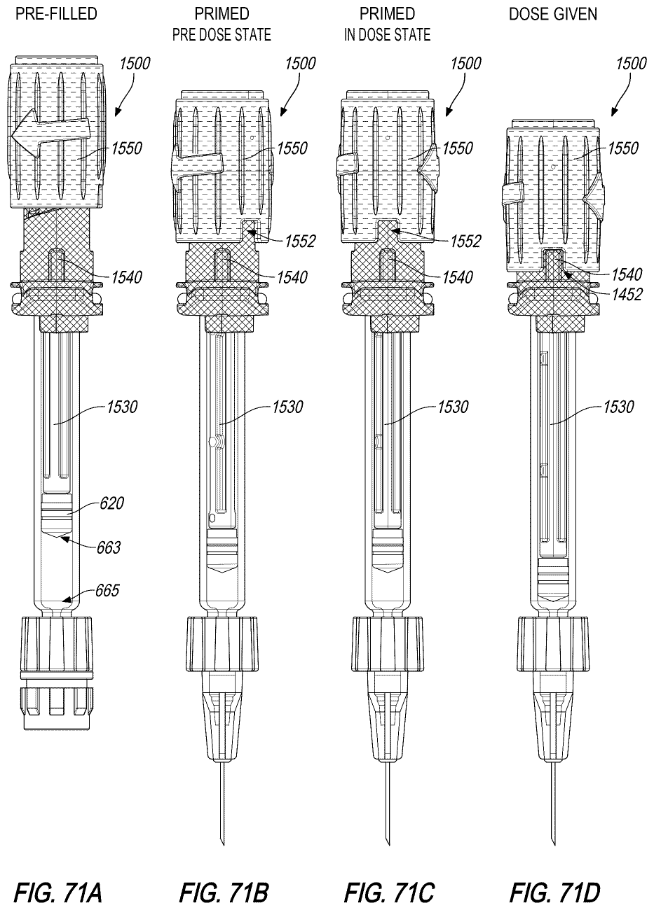

FIGS. 71A, 71B, 71C, and 71D are side views depicting a system 1500 for injecting in various configurations according to some embodiments. The system 1500 includes a plunger member 1530, a finger flange 1540, and a shroud 1550. The plunger member 1530 and the shroud 1550 have the same color, which is unique in the system 1500. The color of the plunger member 1530 and the shroud 1550 is brown compared to the rest of the system 1500, which is dark grey. The plunger member 1530 and the shroud 1550 share the same color to indicate to a user the shroud 1550 rotates to advance the plunger member 1530 to transform the system 1500 from the transport configuration to the primed/injection ready configuration. The shroud also includes a pair of slots 1552 through which the dark grey color of the finger flange 1540 is visible. The slots 1552 indicate to a user the amount of rotation to transform the system 1500 from the transport configuration to the primed/injection ready configuration.

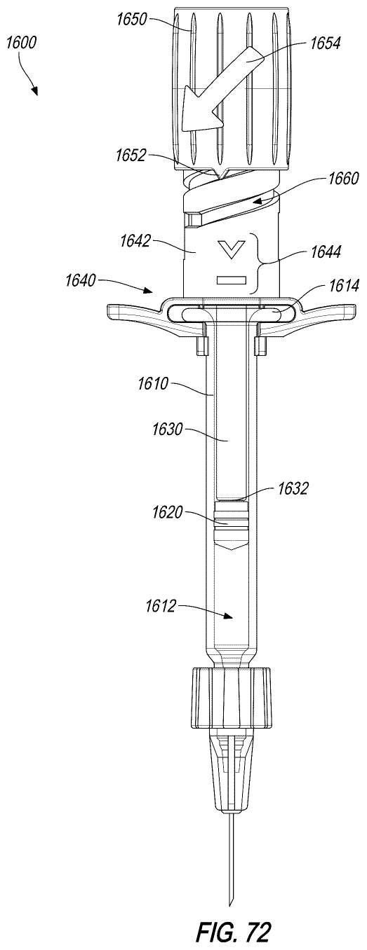

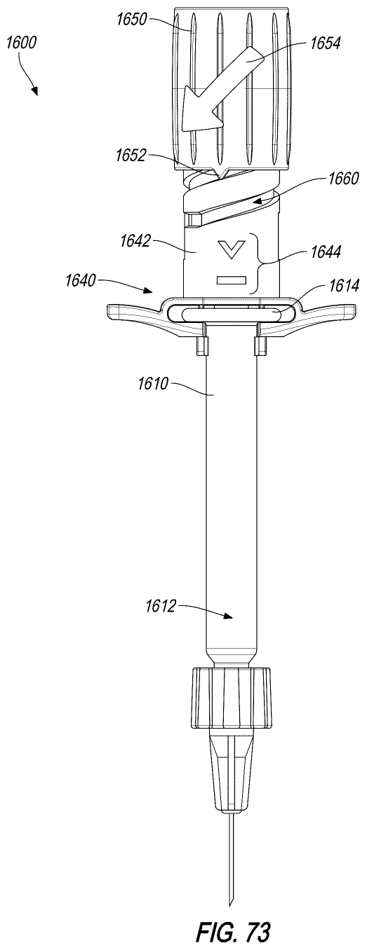

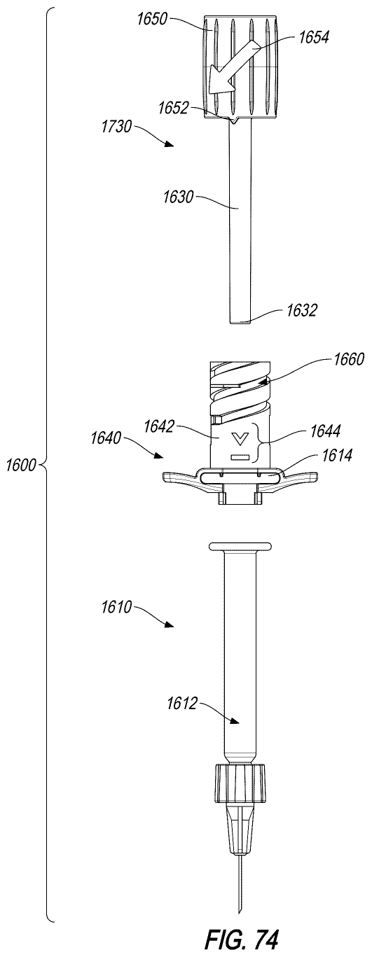

FIGS. 72 and 73 are front views depicting system/syringe assembly 1600 for injecting according to some embodiments. FIG. 74 is an exploded view depicting the system/syringe assembly 1600 for injecting according to some embodiments. The system 1600 includes a syringe body 1610 having proximal and distal ends, a syringe interior 1612, and a syringe flange 1614 at the proximal end thereof. A stopper member 1620 is disposed in the syringe interior 1612 to form a fluid chamber therein. The syringe interior/fluid chamber 1612 contains an injectable fluid (not shown) and some air. A plunger member 1630 is partially disposed in the syringe interior 1612. The plunger member 1630 has a distal end 1632 disposed adjacent to the stopper member 1620. The system 1600 also includes a finger flange 1640 coupled to the syringe flange 1612. The system 1600 further includes a shroud 1650 coupled to the finger flange 1640 and the plunger member 1630.

The plunger member 1630 and the shroud 1650 are one combined part, as such the plunger member 1630 is rotatably and translationally coupled to the shroud 1650. While the distal end 1632 of the plunger member 1630 of the system depicted in FIGS. 72 to 74 has a flat end, in other embodiments the distal end of the plunger member may have a screw type connector to couple the plunger member to the stopper member.

The finger flange 1640 defines a proximally extending member 1642, which has a pair of external threads 1660 defined thereon. The external threads 1660 are double lead threads with about a 5 mm separation there between. The proximally extending member 1642 also defines pair of external indicia 1644 of a configuration of the system 1600 (described below).

The shroud 1650 defines a pointer 1652 at a distal end thereof. The pointer 1652 is configured to cooperate with the external indicia 1644 on the proximally extending member 1642 of the finger flange 1640. The shroud 1650 also defines an external indicia of rotation direction 1654. The shroud 1650 has a knurled external surface to facilitate manual rotation thereof.

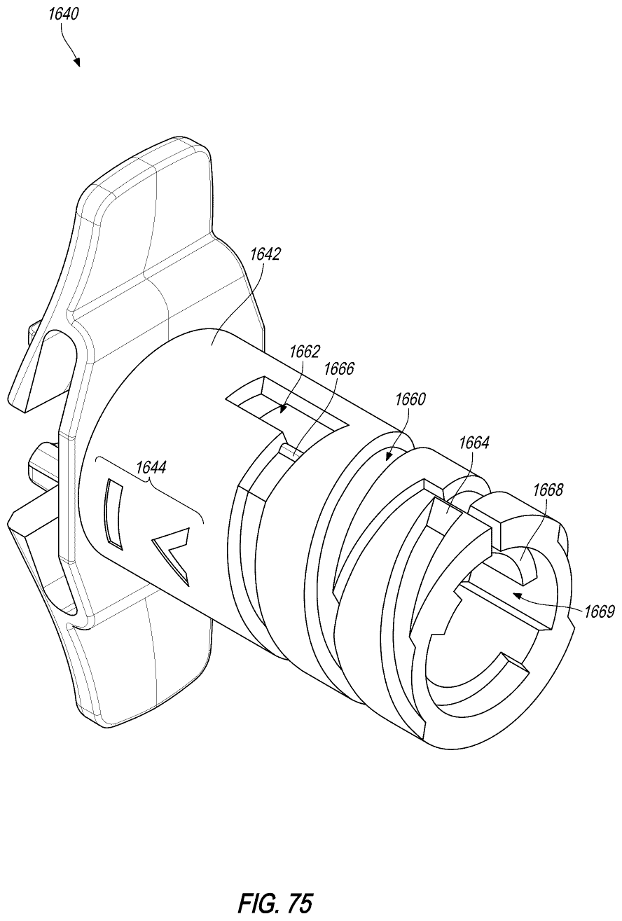

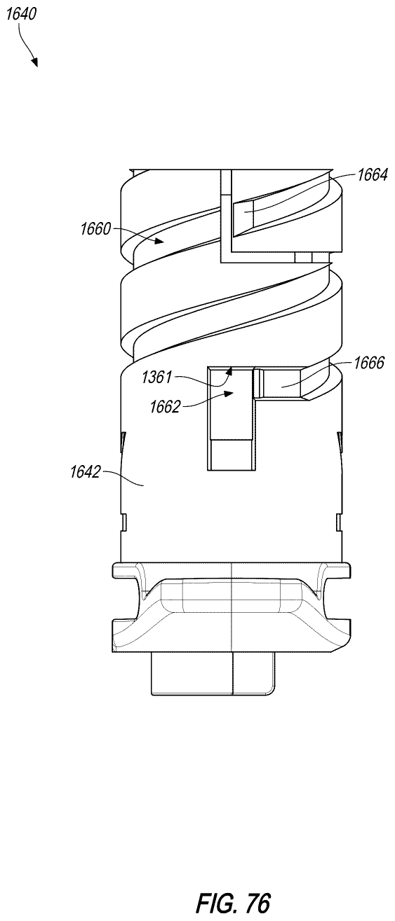

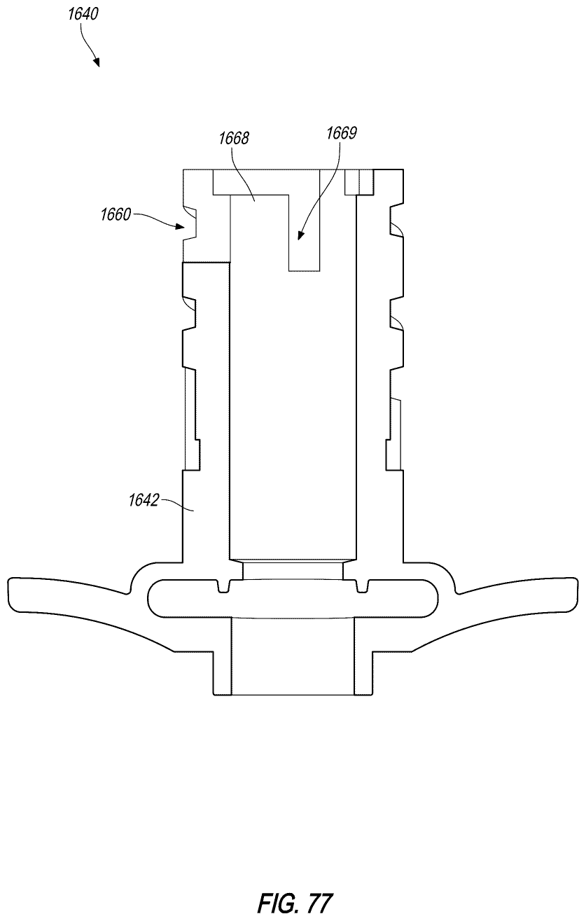

FIGS. 75, 76, and 77 are perspective, side, and longitudinal cross-sectional views depicting a finger flange 1640 for use with systems for injecting, such as the system 1600 depicted in FIGS. 72, 73, and 74, according to some embodiments. The finger flange 1640 defines a proximally extending member 1642, which has a pair of external threads 1660 defined thereon. The external threads 1660 are double lead threads with about a 5 mm separation there between. The proximally extending member 1642 also defines pair of external indicia 1644 of a configuration of the system 1600 (described below). The proximally extending member 1642 further defines a pair of distally extending external longitudinal slot 1662 at respective distal ends of the external threads 1660. Moreover, the proximally extending member 1642 defines a proximal bump 1664 adjacent both proximal ends of the external threads 1660 and a pair of radial bumps 1666 adjacent respective distal ends of the external threads 1660. The radial bumps 1666 separate the distal ends of the external threads 1660 from the proximal ends of the longitudinal slots 1662. In addition, the proximally extending member 1642 defines a pair of inwardly extending arcuate stops 1668 and a pair of distally extending internal longitudinal slots 1669 (see FIGS. 75 and 77). In some embodiments, the proximally extending member may define a pair of proximal bumps adjacent respective proximal ends of the external threads.

While the threaded connection illustrated in the embodiment herein is a double-lead or 2-start thread, the threads may be a single lead or have 3 or more leads. The thread lead or pitch or distance traveled in one thread revolution may be between 2 and 10 mm. In a preferred embodiment, the thread lead is 8.57 mm. The threaded connection may be configured with a thread lead or pitch that makes the thread self-locking, where the coefficient of friction between the finger flange and the shroud is greater than or equal to the tangent of the lead angle of the thread. Where the lead angle of the thread is the inverse tangent of the lead divided by π times the thread diameter. In a self-locking thread, a proximally directed force that the stopper member exerts on the plunger member and the intercoupled shroud during use, manufacturing, or shipment will not rotate and/or translate the threaded shroud and intercoupled plunger member in a proximal direction. During terminal sterilization of a filled and closed syringe body, with the finger flange attached, pressure may build up in the medicine chamber due to the temperature increase. This pressure may cause a proximally directed force to be applied to the stopper member, urging the stopper to move proximally. It is desired not to allow the stopper to move more than the distance between the sealing ribs of the stopper member (see e.g., FIG. 5A) to prevent the exposure of potentially non-sterile surfaces of the stopper member and/or syringe body to the injectable fluid. The self-locking threaded connection between the finger flange and the shroud, and the intercoupled plunger member and stopper member prevents the movement of the stopper member proximally until the temperature of the prefilled syringe body is reduced and the pressure in the syringe interior is reduced. Additionally, during air shipment the filled, stoppered, and assembled syringe bodies may experience a drop in atmospheric pressure external to the syringe bodies. With a drop in external pressure, the injectable fluid and any entrained air bubbles in the syringe body may expand and apply a proximally directed force on the stopper member. It is desired not to allow the stopper member to move more than the distance between the sealing ribs of the stopper member to prevent the exposure of potentially non-sterile surfaces of the stopper member and/or syringe body to the injectable fluid. The self-locking threaded connection between the finger flange and shroud and the intercoupled plunger member and stopper member prevents the movement of the stopper member proximally until the external atmospheric pressure is returned to an amount similar to the pressure in the syringe interior.

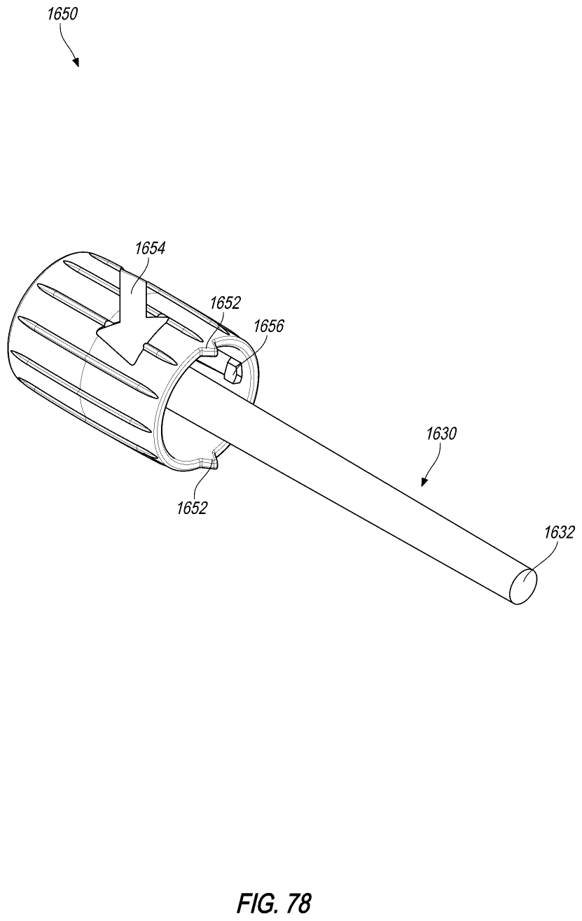

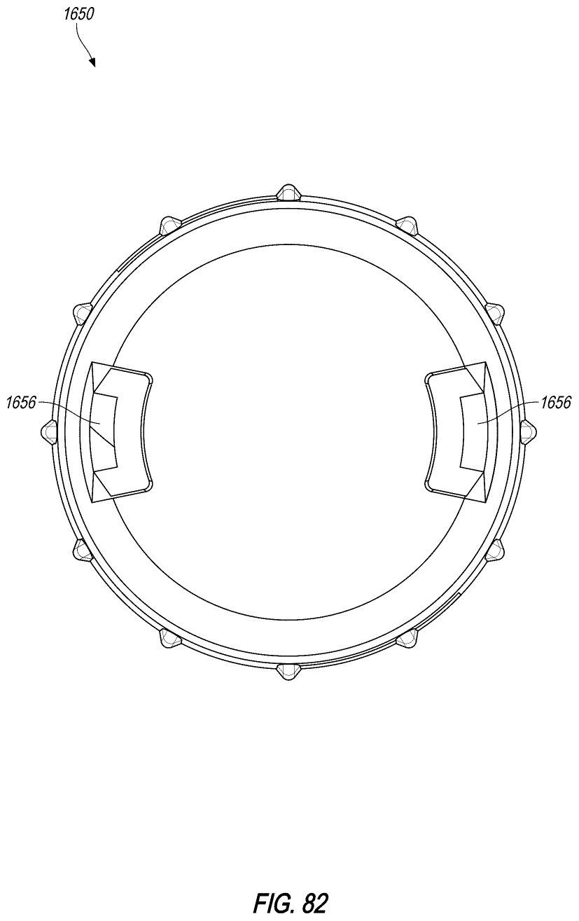

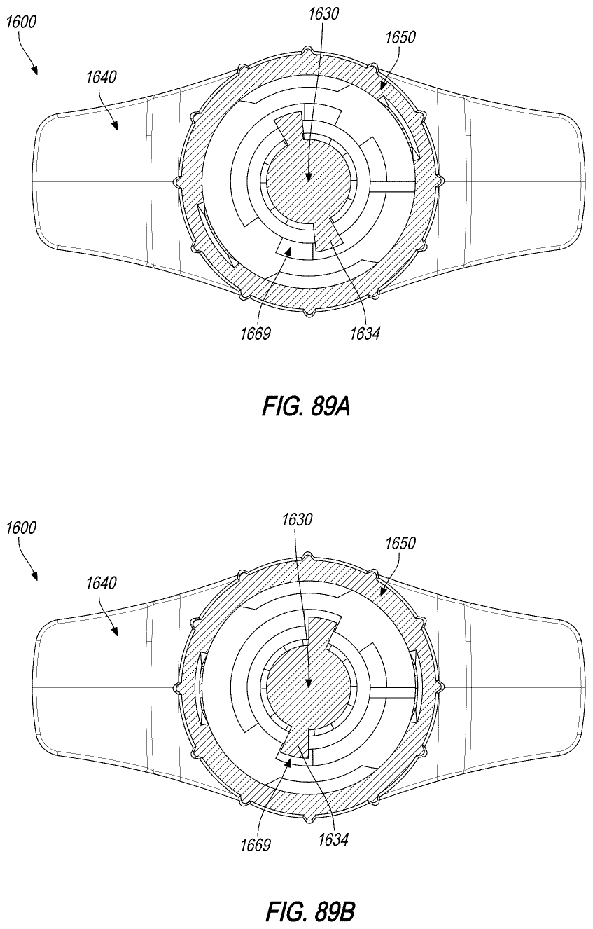

FIGS. 78, 79, 80, 81, and 82 are perspective, longitudinal cross-sectional (along two different axes), and transverse cross-sectional views depicting a shroud 1650 and a plunger member 1630 (except for FIG. 82) for use with systems for injecting, such as the system 1600 depicted in FIGS. 72, 73, and 74, according to some embodiments. The plunger member 1630 and the shroud 1650 are one combined part, as such the plunger member 1630 is rotatably and translationally coupled to the shroud 1650. The shroud 1650 defines a pair of pointers 1652 at a distal end thereof. The pointers 1652 are configured to cooperate with a corresponding pair of external indicia 1644 on the proximally extending member 1642 of the finger flange 1640 (see e.g., FIGS. 89A and 89B, which are described below). The shroud 1650 also defines a pair of external indicia of rotation direction 1654. The shroud 1650 has a knurled external surface to facilitate manual rotation thereof. The shroud also defines a pair of inwardly extending members 1656 configured to be disposed in respective external threads 1660 in the proximally extending member 1642 of the finger flange 1640. The inwardly extending members 1656 are also configured to travel along the external threads 1660 with rotation of the shroud 1650 relative to the finger flange 1640. The inwardly extending members 1656 are further configured to travel along the distally extending external longitudinal slots 1662 with axial translation of the shroud 1650 relative to the finger flange 1640.

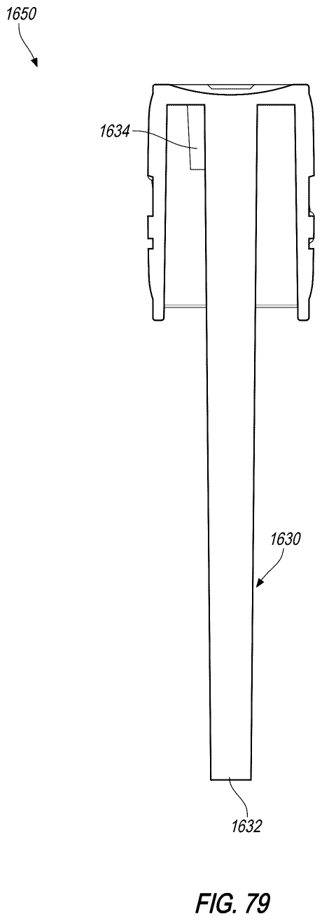

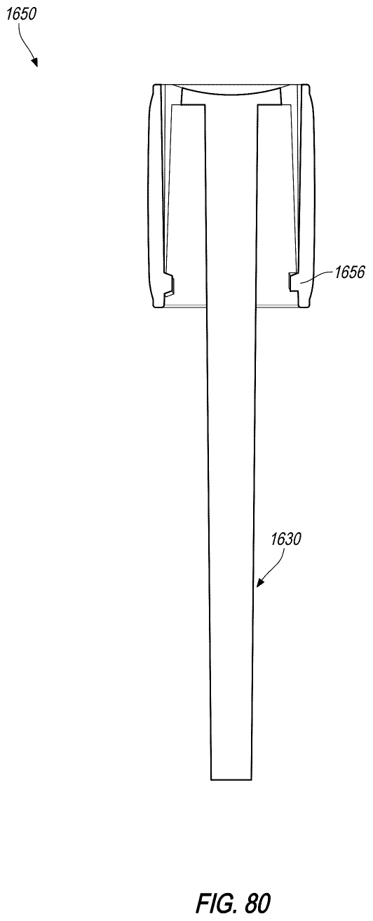

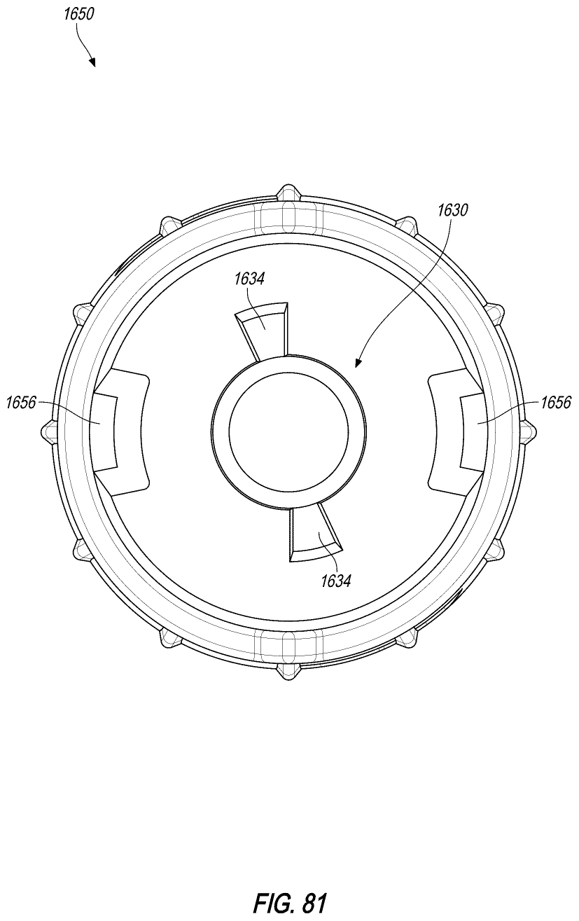

As shown in the longitudinal cross-sectional views in FIGS. 79 and 80, the plunger member 1630 and the shroud 1650 are one combined part, as such the plunger member 1630 is rotatably and translationally coupled to the shroud 1650. As shown in FIG. 81, the plunger member defines a pair of elongate member 1634 outwardly extending from an external surface thereof. The elongate members 1634 are configured to travel along the distally extending internal longitudinal slots 1669 of the proximally extending member 1642 of the finger flange 1640. As shown in FIGS. 81 and 82, the shroud 1650 defines a pair of inwardly extending members 1656 configured to be disposed in respective external threads 1660 in the proximally extending member 1642 of the finger flange 1640.

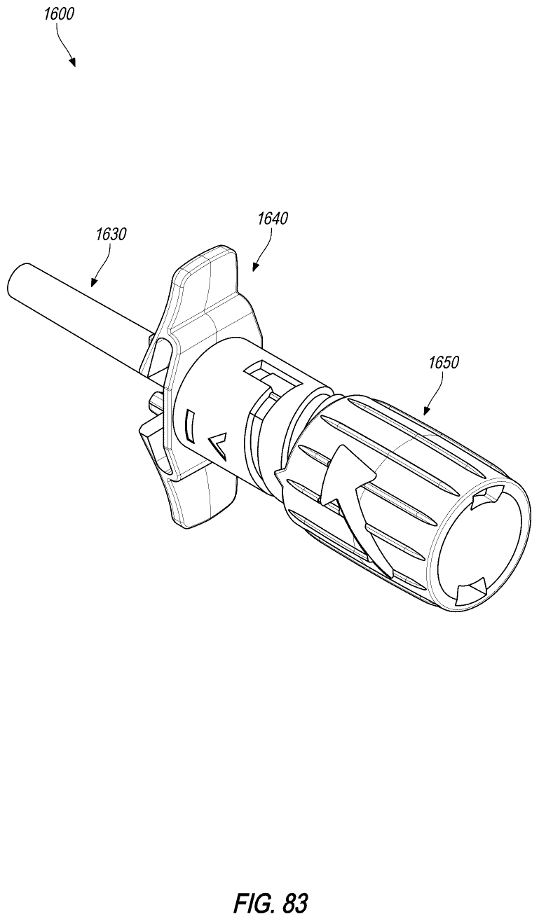

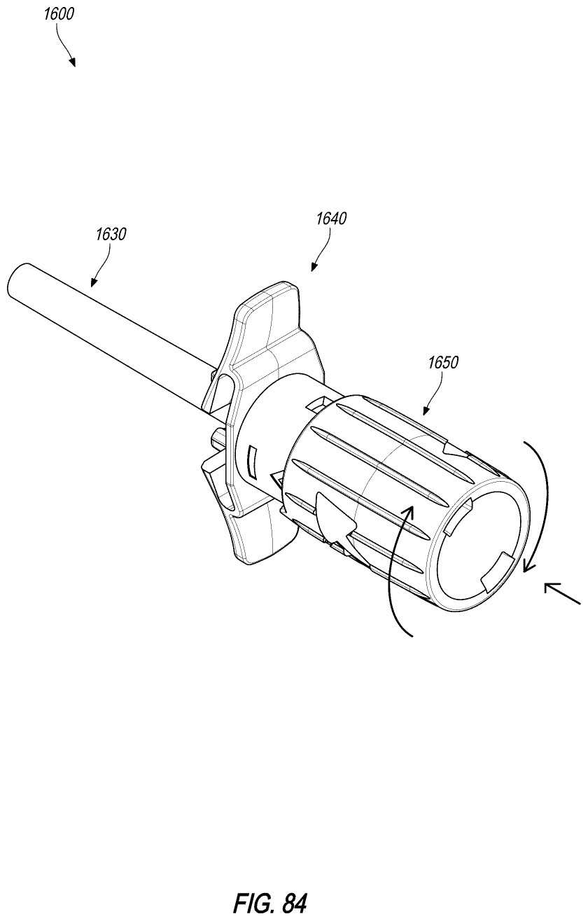

FIGS. 83, 84, 85, and 86 are perspective views depicting a shroud 1650 and a plunger member 1630 mounted on a finger flange 1640 for use with systems for injecting, such as the system 1600 depicted in FIGS. 72, 73, and 74, in various configurations according to some embodiments. In FIG. 83, the system 1600 is in a transport configuration in which the plunger member 1630 and the shroud 1650 are in a proximal most position. Rotating the shroud 1650 and the plunger member 1630 coupled thereto relative to the finger flange 1640 advances both the shroud 1650 in the plunger member 1630 distally relative to the finger flange 1642 transforms the system 1600 into an almost primed/injection ready configuration depicted in FIG. 84. As the plunger member 1630 is advanced distally relative to the finger flange 1642 the system 1600 is positioned in an upward orientation to allow an advancing stopper member (not shown) to inject air from a syringe interior (not shown).

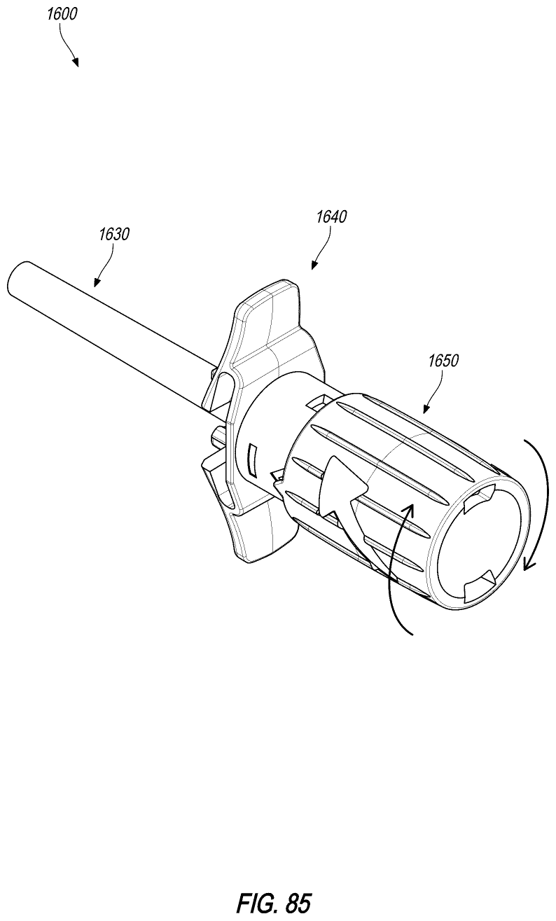

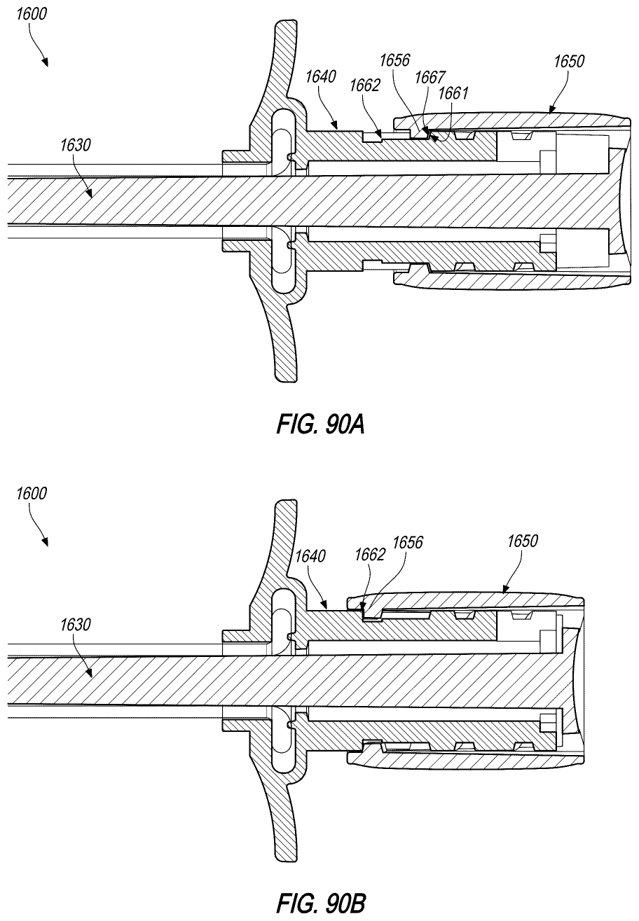

Further rotation of the shroud 1650 and the plunger member 1630 coupled thereto relative to the finger flange 1640 transforms the system 1600 into a primed/injection ready configuration depicted in FIG. 85 by advancing the inwardly extending members 1656 over the radial bumps 1666. When the inwardly extending members 1656 are advanced over the radial bumps 1666, the interaction between the inwardly extending member 1656 and radial bumps 1666 provides a tactile and/or audible indicator that the system 1600 has transitioned to the primed/injection ready configuration depicted in FIG. 85. In the primed/injection ready configuration, proximally facing surfaces 1667 of the inwardly extending members 1656 of the shroud 1650 and the circumferential surface 1661 of the finger flange external thread 1660 are in contact to define the dose start position of the shroud 1650 and the intercoupled plunger member 1630 and stopper member 1620 (see e.g., FIG. 90A).

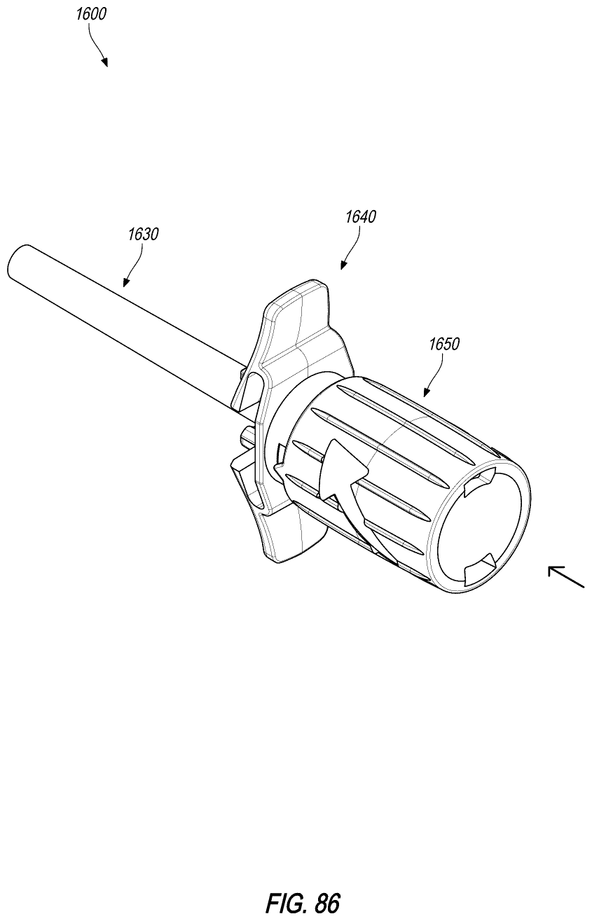

Distally advancing the shroud 1650 and the plunger member 1630 coupled thereto relative to the finger flange 1640 transforms the system 1600 into an injected configuration depicted in FIG. 86. In the injected configuration, the advancing plunger member 1630 has advanced the stopper member 1620 distally to eject a portion of the injectable fluid in the syringe interior 1612. The system 1600 is configured to eject a portion of the injectable fluid that is less than a total amount of the injectable fluid in the syringe interior 1612 to accommodate tolerances in various components of the system 1600.

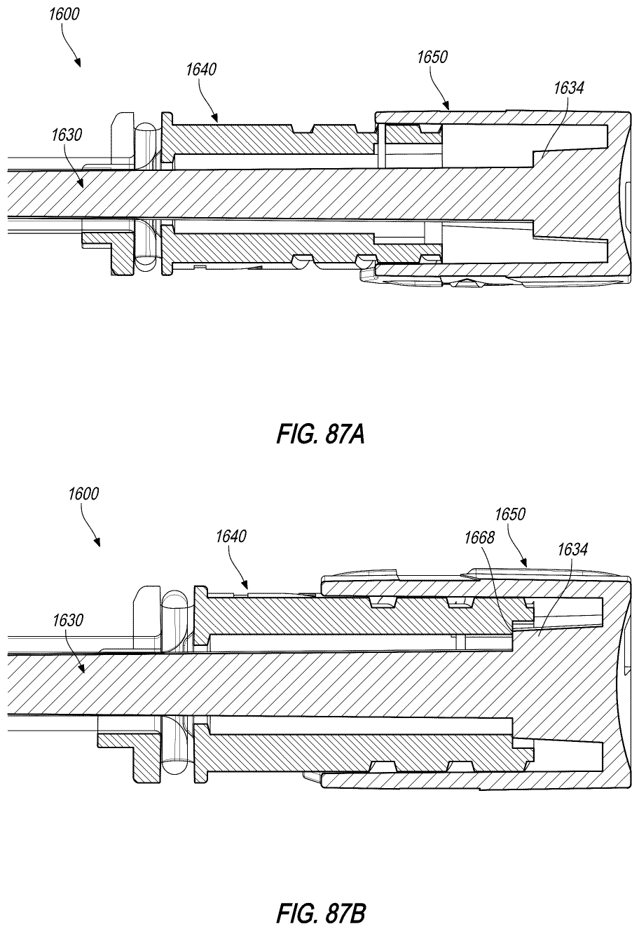

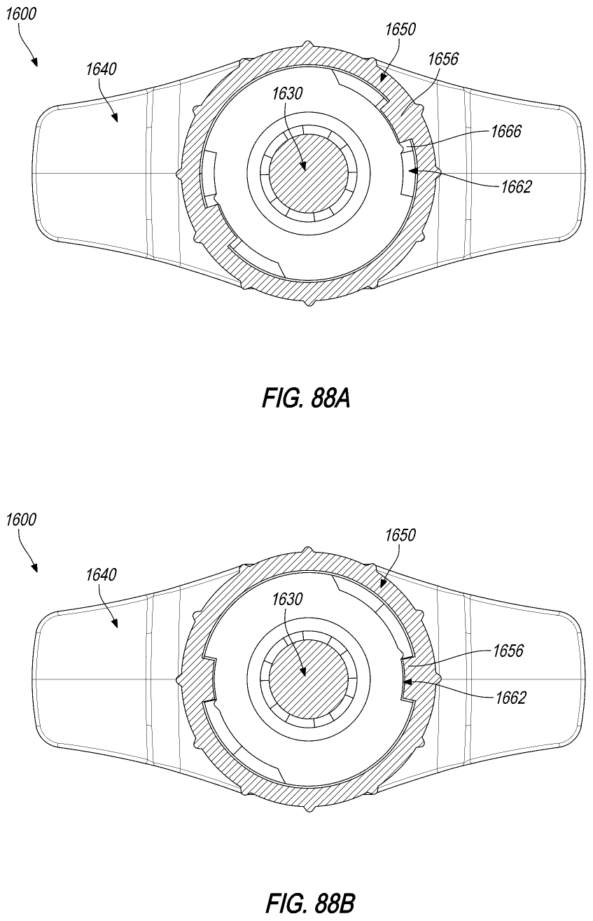

FIGS. 87A, 87B, 88A, 88B, 89A, and 89B are longitudinal and transverse (FIGS. 88A, 88B and 89A, 89B along two different axes) cross-sectional views depicting a shroud 1650 and a plunger member 1630 mounted on a finger flange 1640 for use with systems for injecting, such as the system 1600 depicted in FIGS. 72, 73, and 74, according to some embodiments. In FIGS. 87A, 88A, and 89A, the system 1600 is in the transport configuration depicted in FIG. 83, with the shroud 1650 and the plunger member 1630 in a proximal most position. In FIGS. 87B, 88B, and 89B, the system 1600 has been transformed, by rotation of the shroud 1650 and plunger member 1630 relative to the finger flange 1640, to the primed/injection ready configuration depicted in FIG. 85, with the shroud 1650 and the plunger member 1630 in an intermediate position.

In FIG. 87B, the pair of inwardly extending arcuate stops 1668 interfere with respective elongate members 1634 to stop distal movement (and rotation) of the shroud 1650 and plunger member 1630 in the almost primed/injection ready configuration depicted in FIG. 84. In FIG. 88B, the shroud 1650 is rotated to move the pair of inwardly extending member 1656 over the radial bumps 1666 and into the respective distally extending external longitudinal slots 1662. In FIG. 89B, the shroud is rotated 1650 as in FIG. 88B to move the pair of elongate members 1634 into the pair of distally extending internal longitudinal slots 1669. Moving these members 1656, 1634 into these spaces 1662, 1669 transforms the system 1600 to the primed/injection ready configuration depicted in FIG. 85.

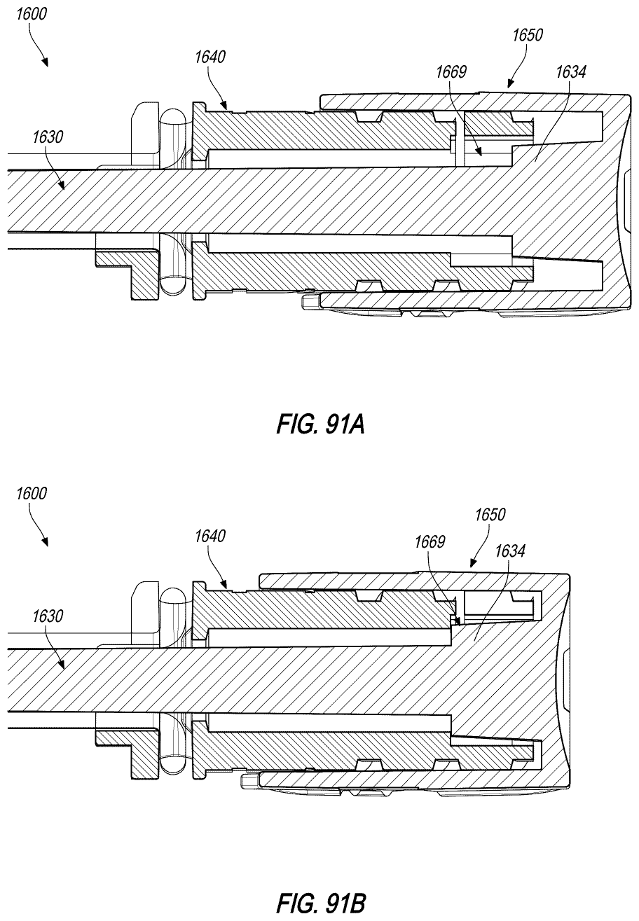

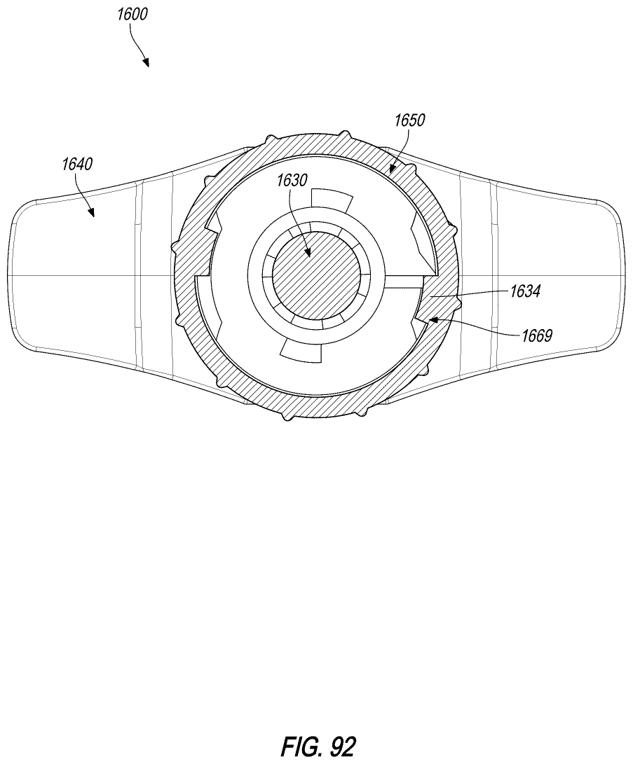

FIGS. 90A, 90B, 91A, 91B, and 92 are longitudinal (FIGS. 90A, 90B, 91A, 91B along two different axes) and transverse cross-sectional views depicting a shroud 1650 and a plunger member 1630 mounted on a finger flange 1640 for use with systems for injecting, such as the system 1600 depicted in FIGS. 72, 73, and 74, according to some embodiments. In FIGS. 90A and 91A, the system 1600 is in the primed/injection ready configuration depicted in FIG. 85, with the shroud 1650 and the plunger member 1630 in an intermediate position. In FIGS. 90B and 91B, the system 1600 has been transformed, by distal movement of the shroud 1650 and the plunger member 1630 relative to the finger flange 1640, to the injected configuration depicted in FIG. 86, with the shroud 1650 and the plunger member 1630 in a distal most position. FIGS. 90A and 90B show the inwardly extending member 1656 traveling along the distally extending external longitudinal slots 1662. FIGS. 91A and 91B show the elongate members 1634 traveling along the distally extending internal longitudinal slots 1669. FIG. 92 shows the pair of elongate members 1634 remaining in the respective pair of distally extending internal longitudinal slots 1669 as the elongate member 1634 move distally along the longitudinal slots 1669.

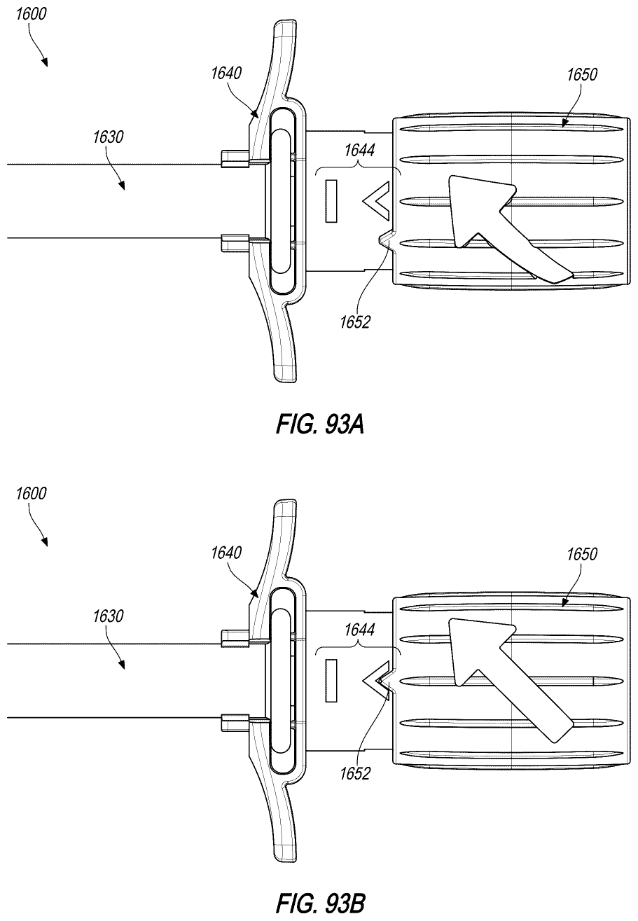

FIGS. 93A and 93B are detailed front views depicting a shroud 1650 and a finger flange 1640 for use with systems for injecting, such as the system 1600 depicted in FIGS. 72, 73, and 74, in two configurations according to some embodiments. In FIG. 93A, the system 1600 is in the almost primed/injection ready configuration depicted in FIG. 84. In this configuration, the pointer 1652 on the shroud 1650 does not align with the external indicia 1644 on the finger flange 1640. In FIG. 93B, the system 1600 is in the primed/injection ready configuration depicted in FIG. 85. In this configuration, the pointer 1652 on the shroud 1650 is aligned with the external indicia 1644 on the finger flange 1640.

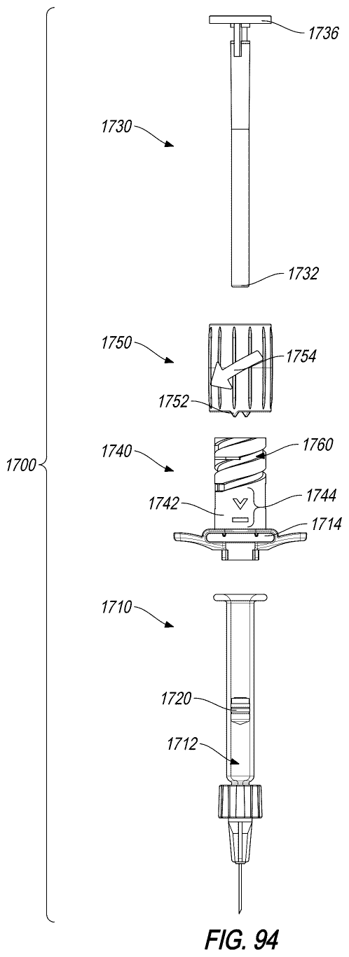

FIG. 94 is an exploded view depicting a system 1700 for injecting according to some embodiments. The system 1700 depicted in FIG. 94 is very similar to the system 1600 depicted in FIGS. 72 to 93B except that the plunger member 1730 is removably coupled to the shroud 1750. Components of system 1700 have similar numbers to corresponding components of system 1600.

The system 1700 includes a syringe body 1710 having proximal and distal ends, a syringe interior 1712, and a syringe flange 1714 at the proximal end thereof. A stopper member 1720 is disposed in the syringe interior 1712 to form a fluid chamber therein. The syringe interior/fluid chamber 1712 contains an injectable fluid (not shown) and some air. A plunger member 1730 is partially disposed in the syringe interior 1712. The plunger member 1730 has a distal end 1732 disposed adjacent to the stopper member 1720. The system 1700 also includes a finger flange 1740 coupled to the syringe flange 1712. The system 1700 further includes a shroud 1750 coupled to the finger flange 1740. The shroud 1750 is removably coupled to the plunger member 1730.

The syringe body 1710, stopper member 1720, and finger flange 1740 are identical to the syringe body 1610, stopper member 1620, and finger flange 1640 of the system 1600 described herein. The plunger member 1730 and shroud 1740 are very similar to the plunger member 1630 and shroud 1640 of the system 1600 described herein. For instance, the plunger member 1730 also has a flat distal end 1732 configured to be disposed adjacent the stopper member 1720. The differences is that the plunger member 1730 is removably coupled to the shroud 1740, and the plunger member 1730 has a thumbpad 1736 at a proximal end thereof.

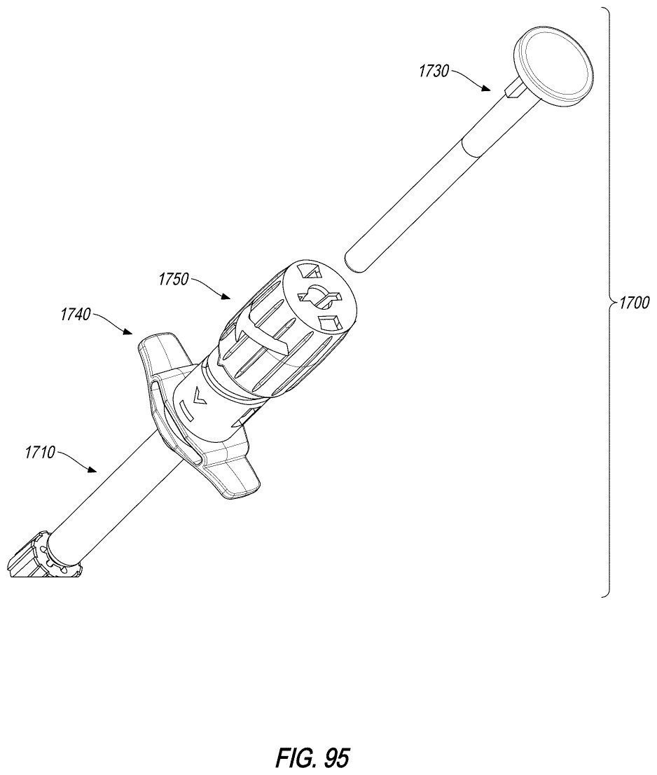

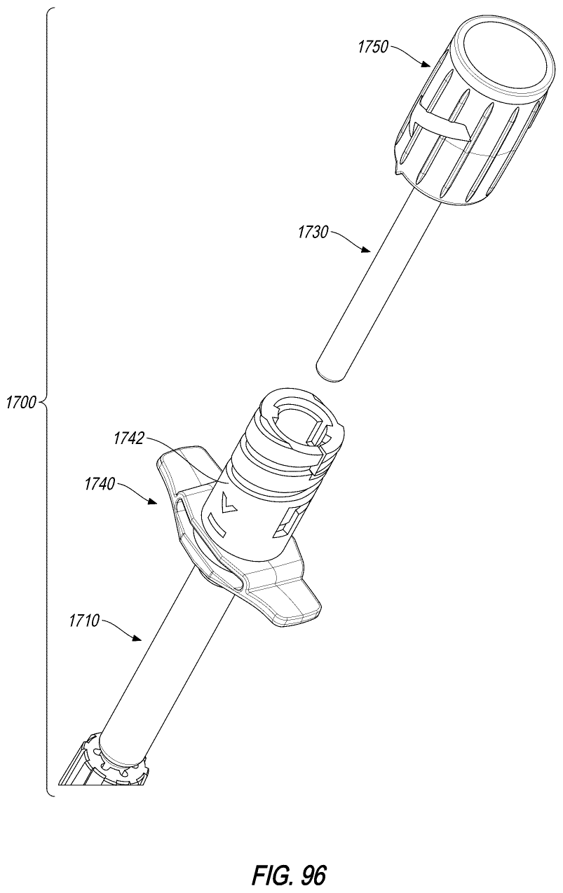

FIGS. 95 and 96 are partially exploded views depicting a system 1700 for injecting according to some embodiments. In FIG. 95, the plunger member 1730 has not been removably coupled to the shroud 1750. In FIG. 96, the plunger member 1730 is removably coupled to the shroud 1750 and ready for assembly onto the proximally extending member 1742 of the finger flange 1740.

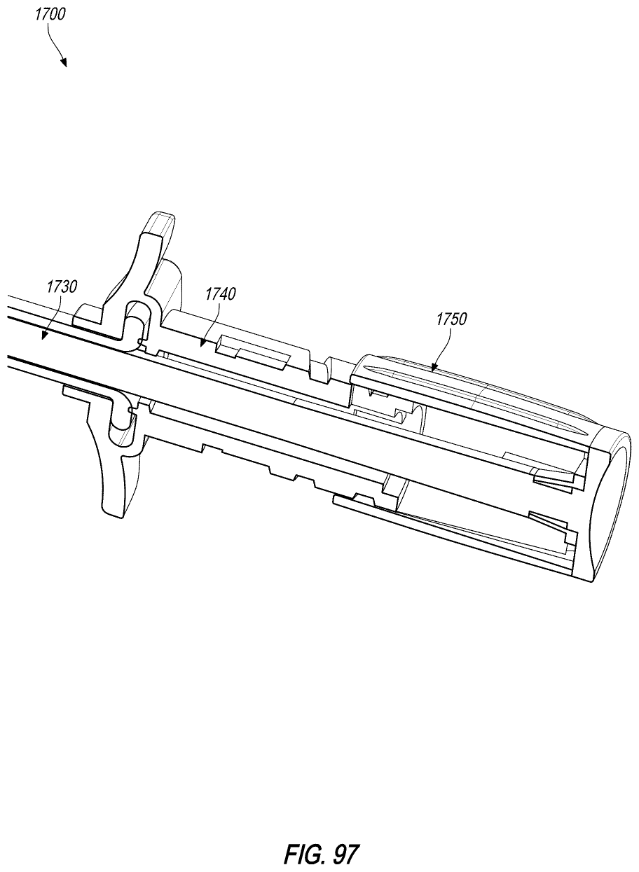

FIG. 97 is a longitudinal cross-sectional view depicting a shroud 1750 and a plunger member 1730 mounted on a finger flange 1740 for use with systems, such as the system 1700 depicted in FIGS. 94, 95, and 96, for injecting according to some embodiments. The system 1700 in FIG. 97 is in a transport configuration similar to the system 1600 depicted in FIGS. 83 and 87A. While the system 1700 is depicted in the transport configuration, the system can also be transformed into a primed/injection ready configuration and an injected configuration similar to the system 1600 depicted in FIGS. 85 and 86 using the same shroud 1750 rotation and translation mechanisms described herein for system 1600.

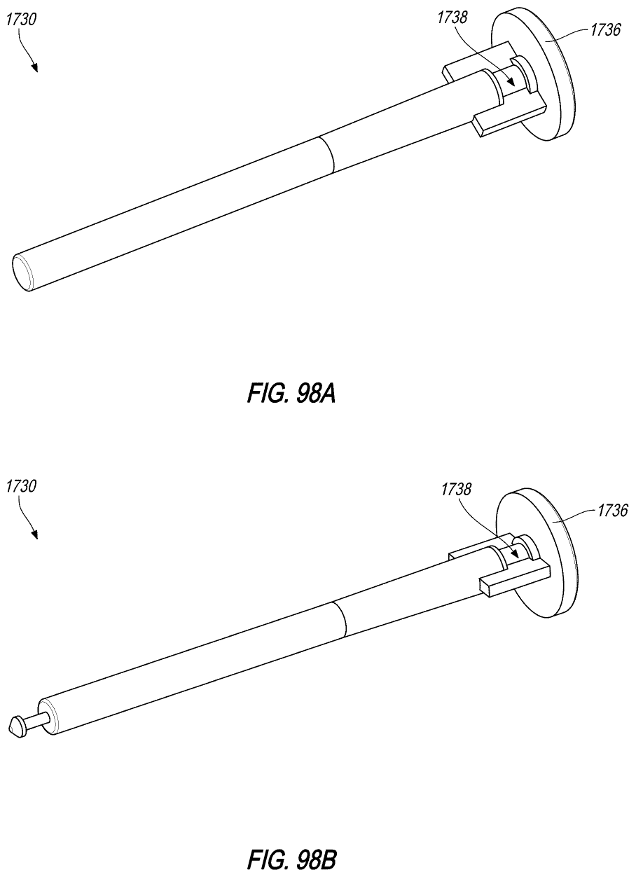

FIGS. 98A and 98B are detailed perspective views depicting a plunger member 1730 for use with systems, such as the system 1700 depicted in FIGS. 94, 29, and 30, for injecting according to some embodiments. The plunger member 1730 defines a pair of grooves 1738 adjacent the thumbpad 1736. The pair of grooves 1738 are configured to removably coupled the plunger member 1730 to the shroud 1750 as described below.

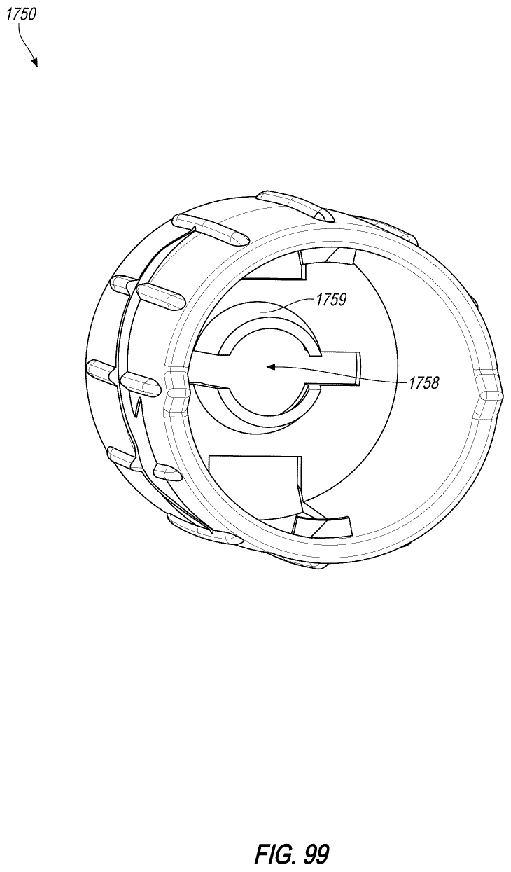

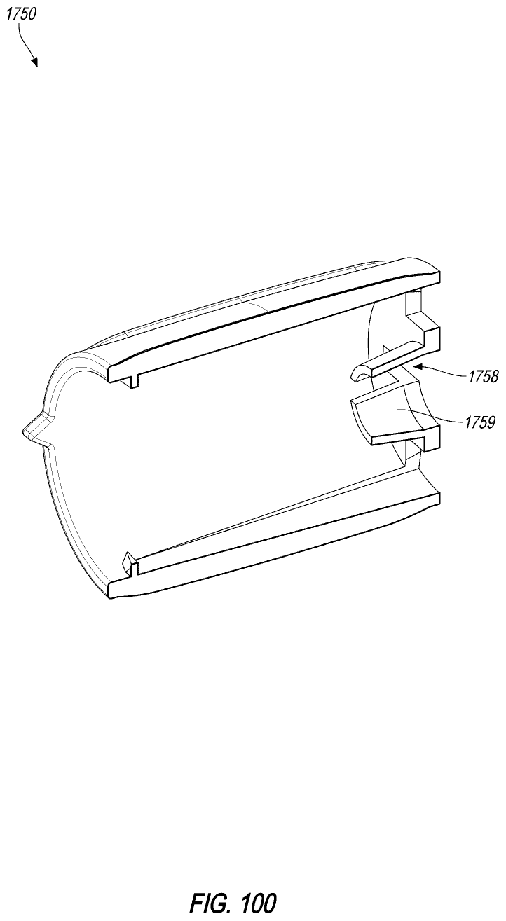

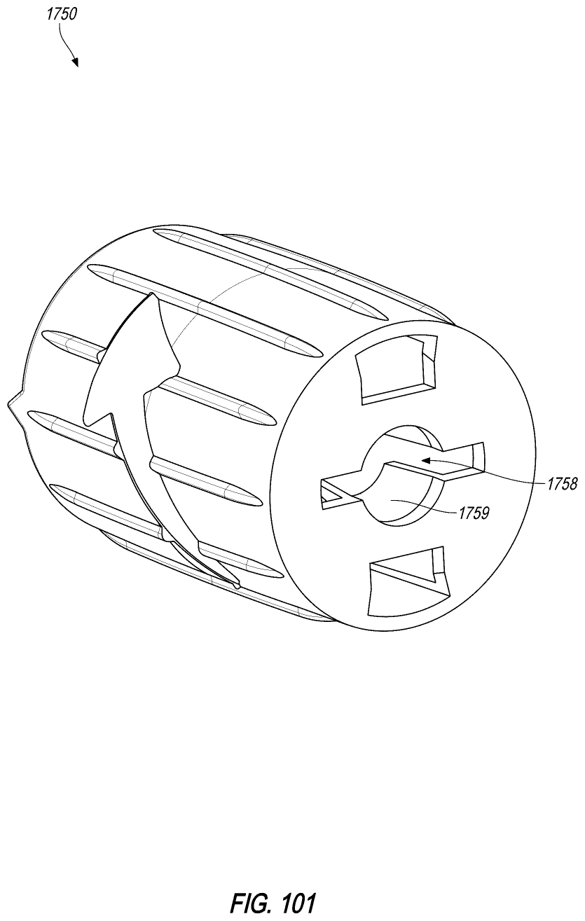

FIGS. 99, 100, and 101 are perspective, longitudinal cross-sectional, and perspective views depicting a shroud 1750 for use with systems, such as the system 1700 depicted in FIGS. 94, 95, and 96, for injecting according to some embodiments. The shroud 1750 defines an opening 1758 at a proximal end thereof. The shroud 1750 also defines a pair of elastically deformable latching members 1759 adjacent the opening 1758.

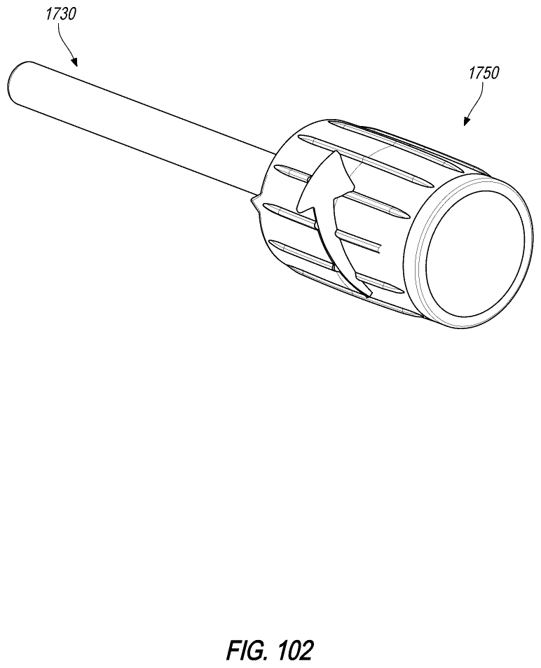

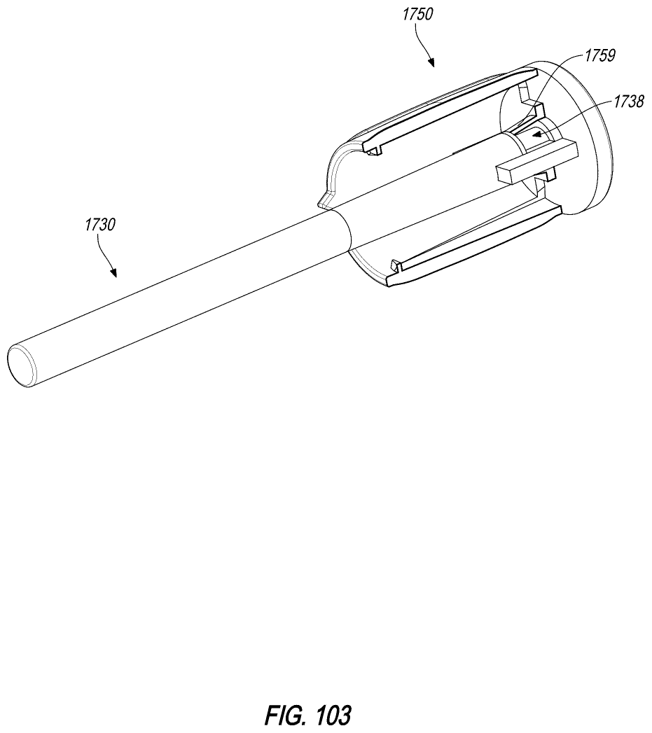

The pair of latching members 1759 elastically deform outward to allowed the plunger member 1730 to pass through and interfere with the pair of grooves 1739 to removably couple the plunger member 1730 to the shroud 1750 as shown in FIGS. 102 and 103. FIGS. 102 and 103 are perspective and longitudinal cross-sectional views depicting a shroud 1750 and a plunger member 1730 for use with systems, such as the system 1700 depicted in FIGS. 94, 95, and 96, for injecting according to some embodiments.

After the plunger member 1730 is removably coupled to the shroud 1750, the system 1700 functions to purge air from the syringe interior 1712 and eject a portion of the fluid in the syringe interior 1712 in a manner identical to the system 1600 and method for using same to perform an injection as depicted in FIGS. 72 to 93B and as described herein.

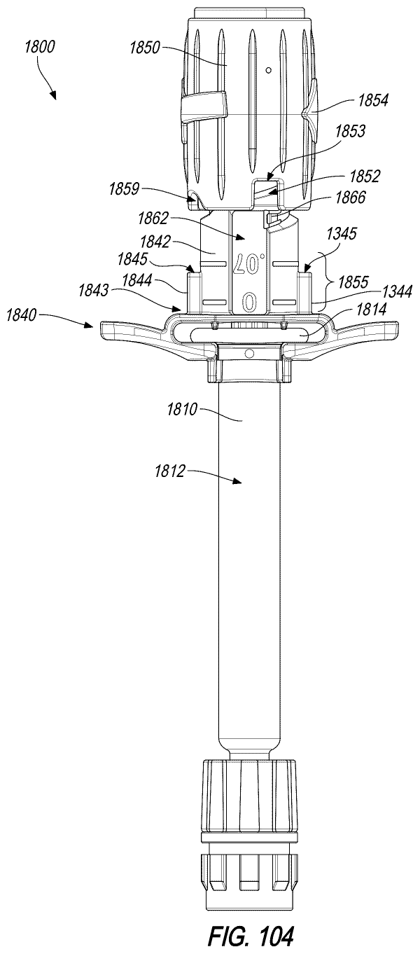

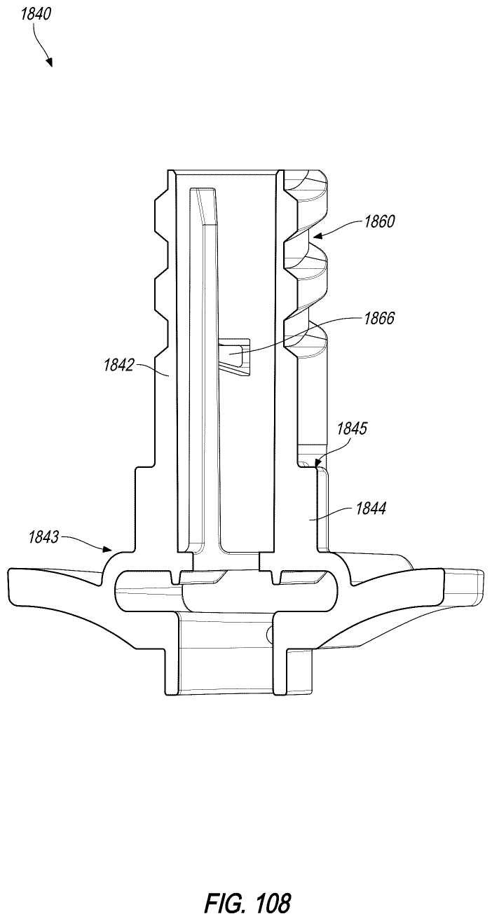

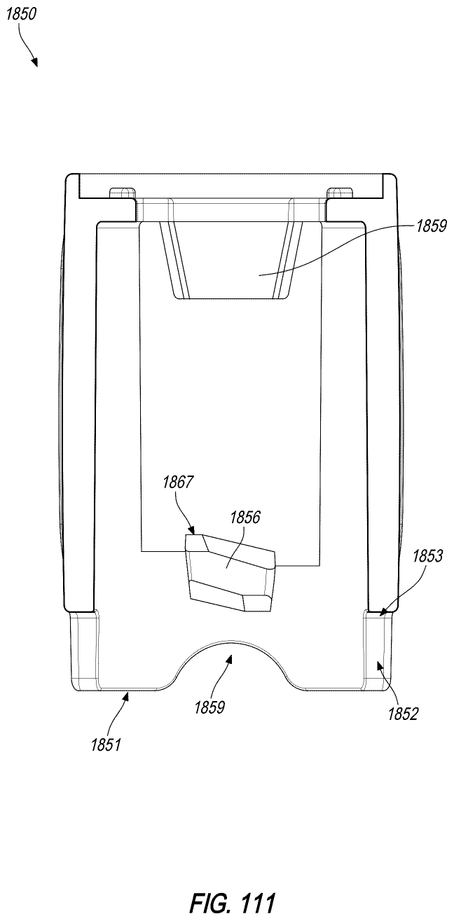

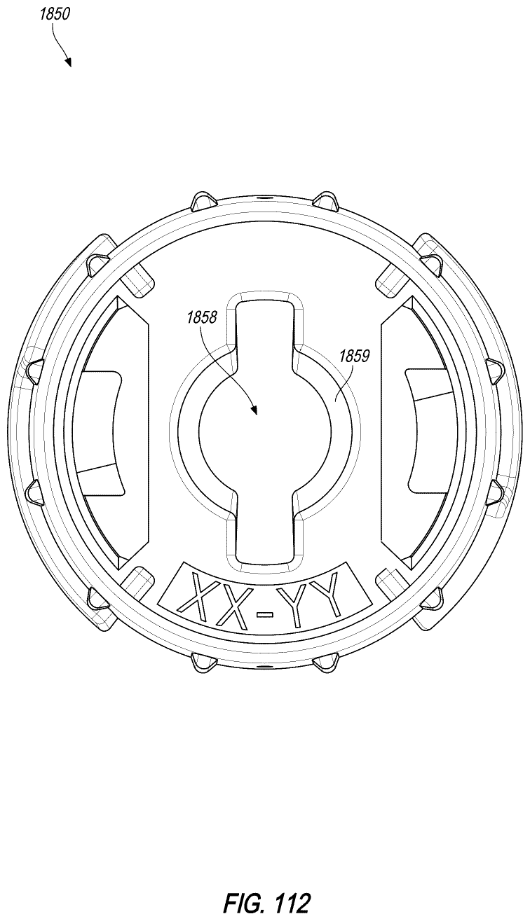

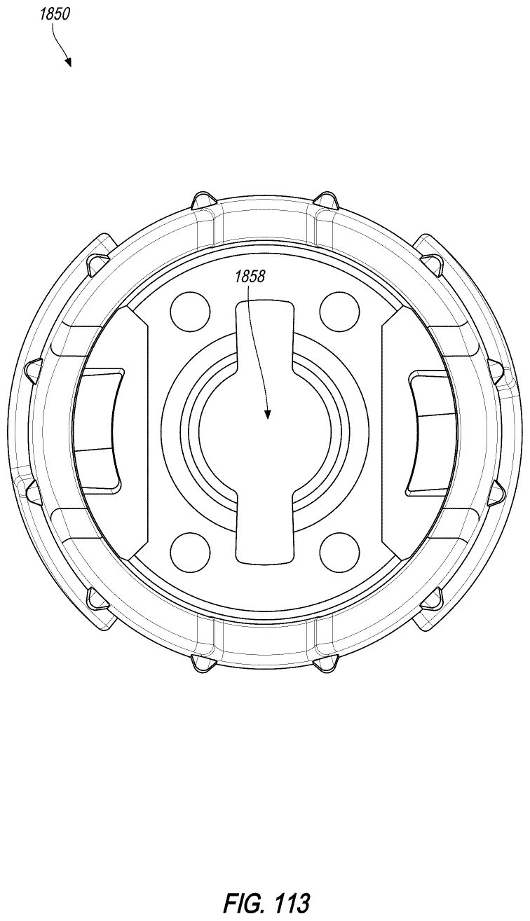

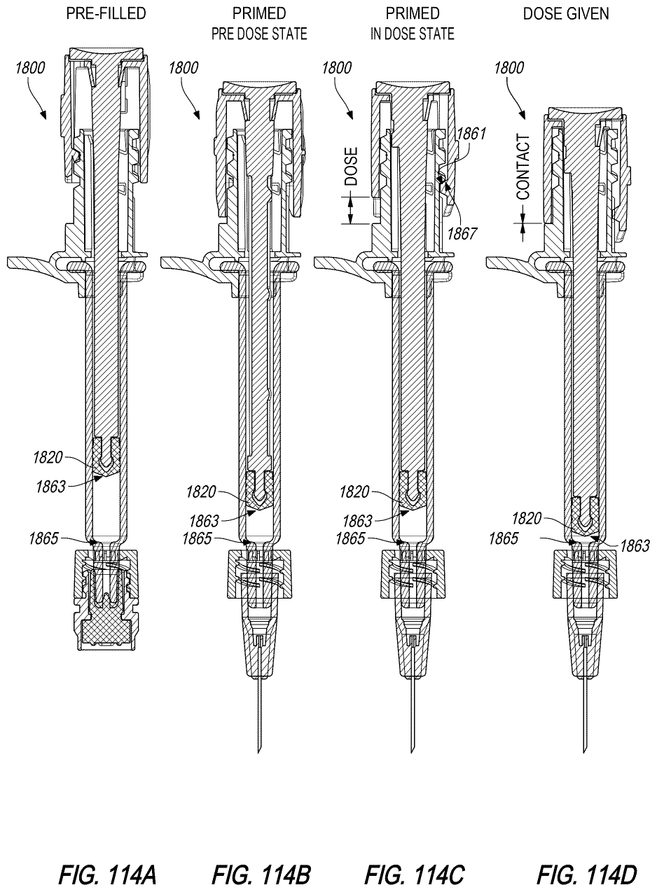

FIGS. 104 to 116D depict various aspects of a system/syringe assembly 1800 for injecting according to some embodiments. The system 1800 depicted in FIGS. 104 to 116D is similar to the system 1300 depicted in FIGS. 54 to 60D, and function in a similar manner to perform an injection. The main difference between the two systems 1300, 1800 is that the proximally extending member 1842 of the finger flange 1840 of the system 1800 depicted in FIGS. 104 to 116D includes external indicia 1855 of a configuration of the system 1800 and its transitions between the primed/injection ready configuration (see e.g., FIGS. 114C, 115C, 116C) and the injected configuration (see e.g., FIGS. 114D, 115D, 116D) in and adjacent to the distally extending external longitudinal slot 1862 thereof.

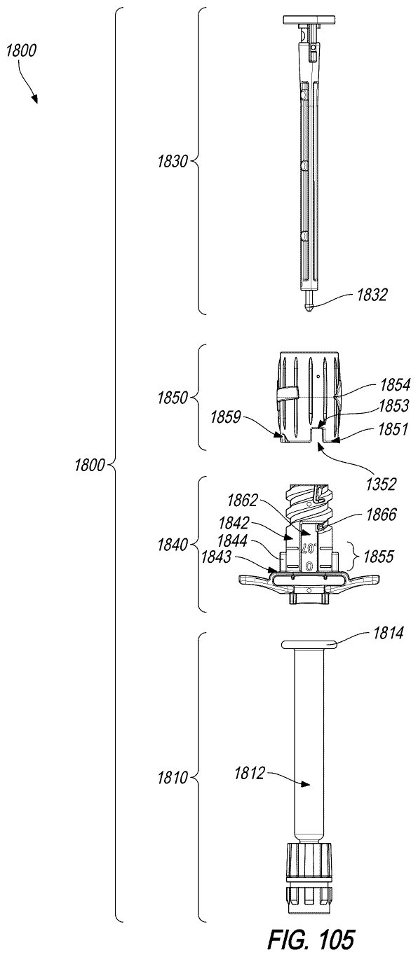

FIG. 104 is a front view depicting a system/syringe assembly 1800 for injecting according to some embodiments. FIG. 105 is an exploded view depicting the system/syringe assembly 1800 for injecting according to some embodiments. The system 1800 includes a syringe body 1810 having proximal and distal ends, a syringe interior 1812, and a syringe flange 1814 at the proximal end thereof. A stopper member 1820 is disposed in the syringe interior 1812 to form a fluid chamber therein. The syringe interior/fluid chamber 1812 contains an injectable fluid (not shown) and some air. A plunger member 1830 is partially disposed in the syringe interior 1812. The plunger member 1830 has a distal end 1832 disposed adjacent to the stopper member 1820. The system 1800 also includes a finger flange 1840 coupled to the syringe flange 1812. The system 1800 further includes a shroud 1850 coupled to the plunger member 1830 and movable relative to the finger flange 1840.

The plunger member 1830 is rotatably and translationally coupled to the shroud 1850. While the distal end 1832 of the plunger member 1830 of the system depicted in FIG. 62 has an interference type connector for a stopper member (not shown), in other embodiments the distal end of the plunger member may have a screw type connector to couple the plunger member to the stopper member or a flat end that only pushes stopper members distally without the ability to pull stopper members proximally.

The finger flange 1840 defines a proximally extending member 1842, which has a pair of external threads 1860 defined thereon. The external threads 1860 are double lead threads with a thread lead of about 8.5 mm, which. yields about a 5 mm pitch or separation between adjacent thread crests. The proximally extending member 1842 also defines a pair of distally extending external longitudinal slot 1862 at respective distal ends of the external threads 1860. The finger flange 1840 defines a flange proximally facing surface 1843 and a pair of flange elongate members 1844 proximally extending from the flange proximally facing surface 1843. While a pair of flange elongate members 1844 are shown, alternative embodiments may have a single flange elongate member, or 3 or more flange elongate members. The finger flange external thread 1860 defines a circumferential surface 1861 at the distal end thereof.

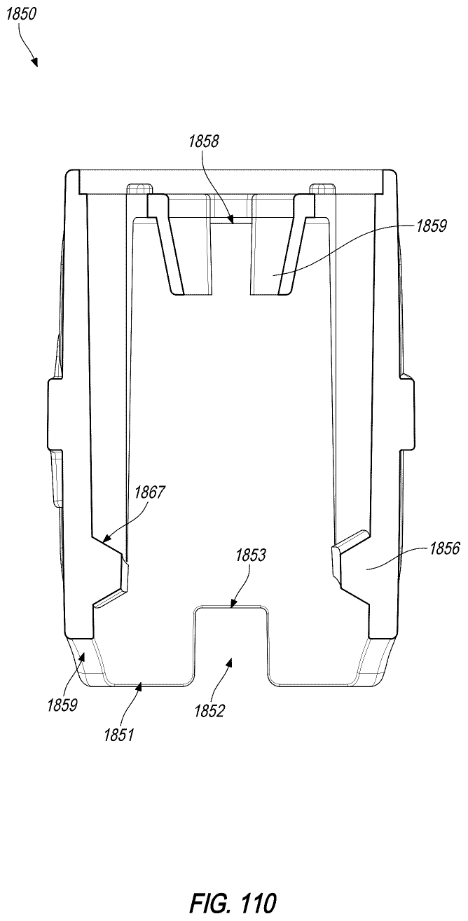

The shroud 1850 defines a shroud distally facing surface 1851 at a distal end thereof configured to interfere with a proximally facing surface 1845 of the flange elongate member 1844 to limit distal movement of the shroud 1850 to a first longitudinal position on the finger flange 1840 when the shroud 1850 is in a first rotational state (see e.g., FIGS. 114C, 115C, 116C) relative to the finger flange 1840. The shroud 1850 is also defines one or more inwardly extending members 1856, each defining a proximally facing surface 1867 (see e.g., FIGS. 110, 111, and 114C). The shroud 1850 also defines a slot 1852 extending proximally from the distally facing surface 1851 and configured to receive the flange elongate member 1844 to allow distal movement of the shroud 1840 distally beyond the first longitudinal position on the finger flange 1850 when the shroud 1840 is in a second rotational state (see e.g., FIGS. 114D, 115D, 116D) relative to the finger flange 1840. The slot 1852 has a distally facing surface 1853. While two slots 1852 are shown, alternative embodiments may have one slot or 3 or more slots. The shroud 1850 further defines a partial window 1859 and an external indicia of rotation direction 1854. Moreover, he shroud 1850 has a knurled external surface to facilitate manual rotation thereof.

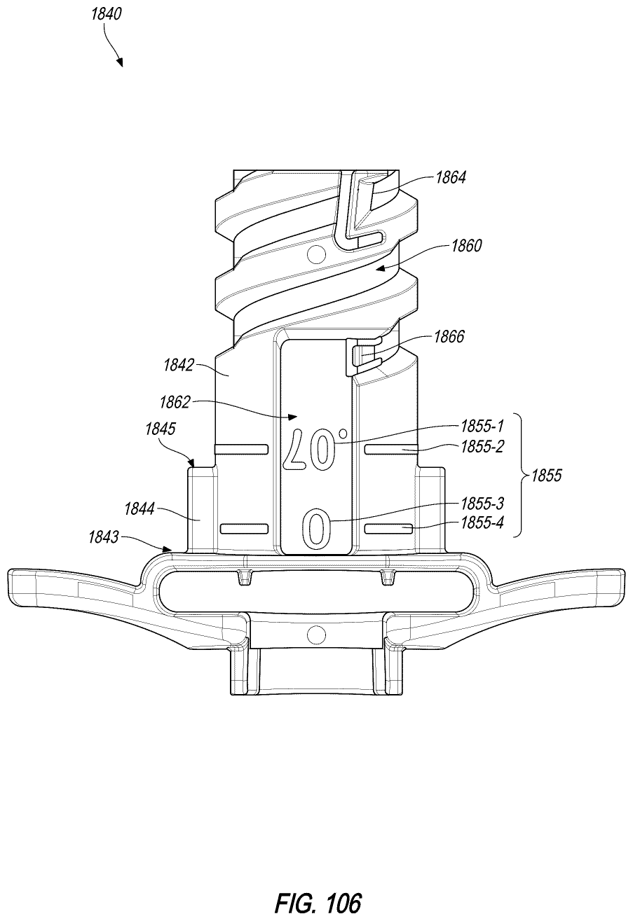

FIGS. 106, 107, and 108 are front, perspective, and cutaway perspective views depicting a finger flange 1840 for use with systems for injecting, such as the system 1800 depicted in FIGS. 104 and 105, according to some embodiments. The finger flange 1840 defines a flange proximally facing surface 1843 and a proximally extending member 1842, which has a pair of external threads 1860 defined thereon. The external threads 1860 are double lead threads with about a 5 mm separation there between. The proximally extending member 1842 also defines a pair of flange elongate members 1844, each having a flange elongate member proximally facing surface 1845. The proximally extending member 1842 further defines a pair of distally extending external longitudinal slot 1862 at respective distal ends of the external threads 1860. Moreover, the proximally extending member 1842 further defines a pair of proximal bumps 1864 adjacent respective proximal ends of the external threads 1860 and a pair of deflectable detents 1866 adjacent respective distal ends of the external threads 1860.

While the threaded connection illustrated in the embodiment herein is a double-lead or 2-start thread, the threads may be a single lead or have 3 or more leads. The thread lead or pitch or distance traveled in one thread revolution may be between 2 and 10 mm. In a preferred embodiment, the thread lead is 8.57 mm. The threaded connection may be configured with a thread lead or pitch that makes the thread self-locking, where the coefficient of friction between the finger flange and the shroud is greater than or equal to the tangent of the lead angle of the thread. Where the lead angle of the thread is the inverse tangent of the lead divided by π times the thread diameter. In a self-locking thread, a proximally directed force that the stopper member exerts on the plunger member and the intercoupled shroud during use, manufacturing, or shipment will not rotate and/or translate the threaded shroud and intercoupled plunger member in a proximal direction. During terminal sterilization of a filled and closed syringe body, with the finger flange attached, pressure may build up in the medicine chamber due to the temperature increase. This pressure may cause a proximally directed force to be applied to the stopper member, urging the stopper to move proximally. It is desired not to allow the stopper to move more than the distance between the sealing ribs of the stopper member (see e.g., FIG. 5A) to prevent the exposure of potentially non-sterile surfaces of the stopper member and/or syringe body to the injectable fluid. The self-locking threaded connection between the finger flange and the shroud, and the intercoupled plunger member and stopper member prevents the movement of the stopper member proximally until the temperature of the prefilled syringe body is reduced and the pressure in the syringe interior is reduced. Additionally, during air shipment the filled, stoppered, and assembled syringe bodies may experience a drop in atmospheric pressure external to the syringe bodies. With a drop in external pressure, the injectable fluid and any entrained air bubbles in the syringe body may expand and apply a proximally directed force on the stopper member. It is desired not to allow the stopper member to move more than the distance between the sealing ribs of the stopper member to prevent the exposure of potentially non-sterile surfaces of the stopper member and/or syringe body to the injectable fluid. The self-locking threaded connection between the finger flange and shroud and the intercoupled plunger member and stopper member prevents the movement of the stopper member proximally until the external atmospheric pressure is returned to an amount similar to the pressure in the syringe interior.

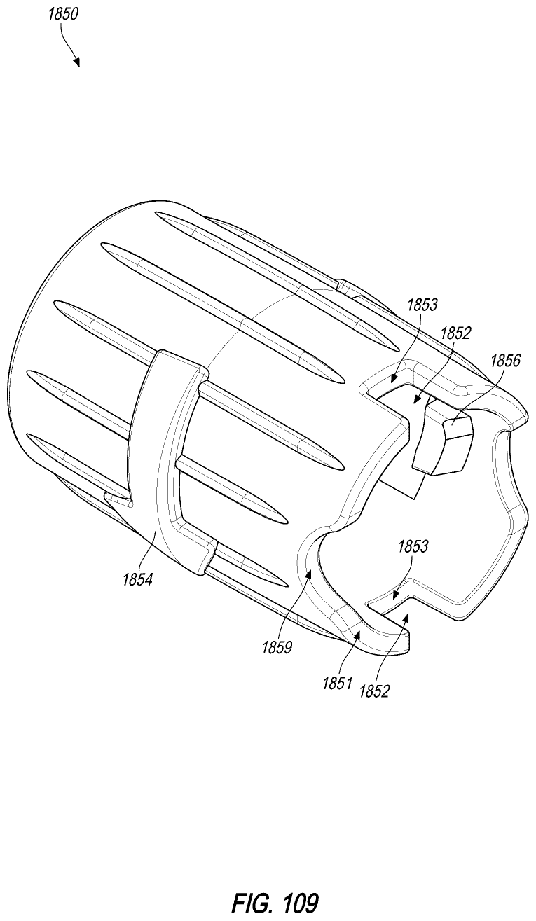

The deflectable detents 1866 are configured to cooperate with respective inwardly extending members 1856 to control movement of the inwardly extending members 1856 between the external thread 1860 and the distally extending external longitudinal slot 1862. The deflectable detents 1866 separate the distal ends of the external threads 1860 from the proximal ends of the longitudinal slots 1862. During the transition from the almost primed configuration (see e.g., FIGS. 114B, 115B, 116B) to the primed/injection ready configuration (see e.g., FIGS. 114C, 115C, 116C), the deflectable detents 1866 elastically deform with application of a deflecting force (e.g., by respective inwardly extending members 1856; see e.g., FIGS. 109-111) in a radial direction to allow the inwardly extending members 1856 to move from respective external thread 1860 to respective distally extending external longitudinal slot 1862. The deflectable detents 1866 return to respective original positions after the deflecting force is removed therefrom in the primed/injection ready configuration (see e.g., 114C, 115C, 116C) and the injected configuration (see e.g., 114D, 115D, 116D). The deflectable detents 1866 returning to the respective original/neutral positions prevents the inwardly extending member 1856 from moving from the distally extending external longitudinal slot 1862 to the external thread 1860. The deflectable detents 1866 may be biased to return to the respective original/neutral positions. The deflectable detents 1866 may also be self-energized (e.g., in response to a force applied by respective inwardly extending members 1856) to return to the respective original/neutral positions. The deflectable detents 1866 may be configured to cooperate with respective inwardly extending members 1856 to generate a tactile and/or audible indicator when the system 1800 transitions from the almost primed configuration (see e.g., FIGS. 114B, 115B, 116B) to the primed/injection ready configuration (see e.g., FIGS. 114C, 115C, 116C). The release/return of the deflectable detents 1866 to the respective original/neutral position may generate audible and/or tactile cues or feedback to the user to indicate that the shroud 1850 has rotated into the dose start position and the injection system 1800 is in the primed/injection ready configuration and ready for the user to inject fluid into the patient.

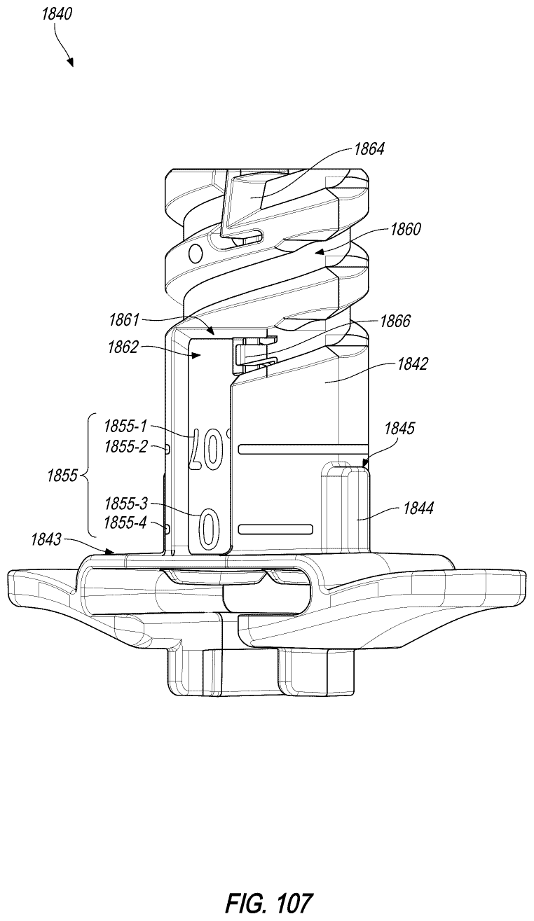

FIGS. 106 and 107 show, in detail, external indicia 1855 of a configuration of the system 1800 in and adjacent to the distally extending external longitudinal slot 1862 on the proximally extending member 1842 of the finger flange 1840. The external indicia 1855 include a primed/injection ready configuration indicia 1855-1, a primed/injection ready configuration indicia line 1855-2 disposed adjacent the primed/injection ready configuration indicia 1855-1, an injected configuration indicia 1855-3, and an injected configuration indicia line 1855-4 disposed adjacent the injected configuration indicia 1855-3. These external indicia 1855-1, 1855-2, 1855-3, 1855-4 of a configuration of the system 1800 may be etched or otherwise imprinted onto the proximally extending member 1842 of the finger flange 1840 with a laser or other imprinting techniques.

When the system 1800 depicted in FIGS. 104 to 116D is in the primed/injection ready configuration (see e.g., FIGS. 114C, 115C, 116C), distal movement of a shroud 1850 relative to a finger flange 1840 is limited to a first longitudinal position by interference between a pair of inwardly extending members 1856 and corresponding external threads 1860 in a proximally extending member 1842 of the finger flange 1840. In the primed/injection ready configuration, the proximally facing surfaces 1867 of the inwardly extending members 1856 and the circumferential surface 1861 of the finger flange external thread 1860 are in contact to define the dose start position of the shroud 1850 and the intercoupled plunger member 1830 and stopper member 1820. In the injected configuration (see e.g., FIGS. 114D, 115D, 116D), a distally facing surface 1853 of the slot 1852 in the shroud 1850 (see FIGS. 105 and 109-111) is configured to interfere with a flange proximally facing surface 1845 of the flange elongate member 1844 of the finger flange 1840 (see FIGS. 104-108) to limit distal movement of the shroud 1850 relative to the finger flange 1840 to a second longitudinal position. In the injected configuration, the pair of inwardly extending members 1856 are not in contact with respective distal ends of the distally extending external longitudinal slots 1862 (see e.g., FIG. 69D).

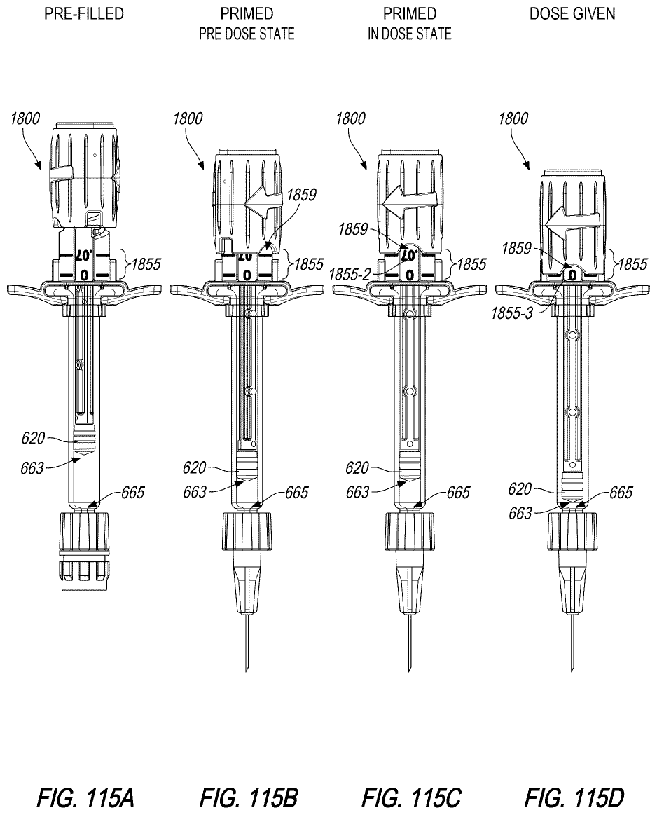

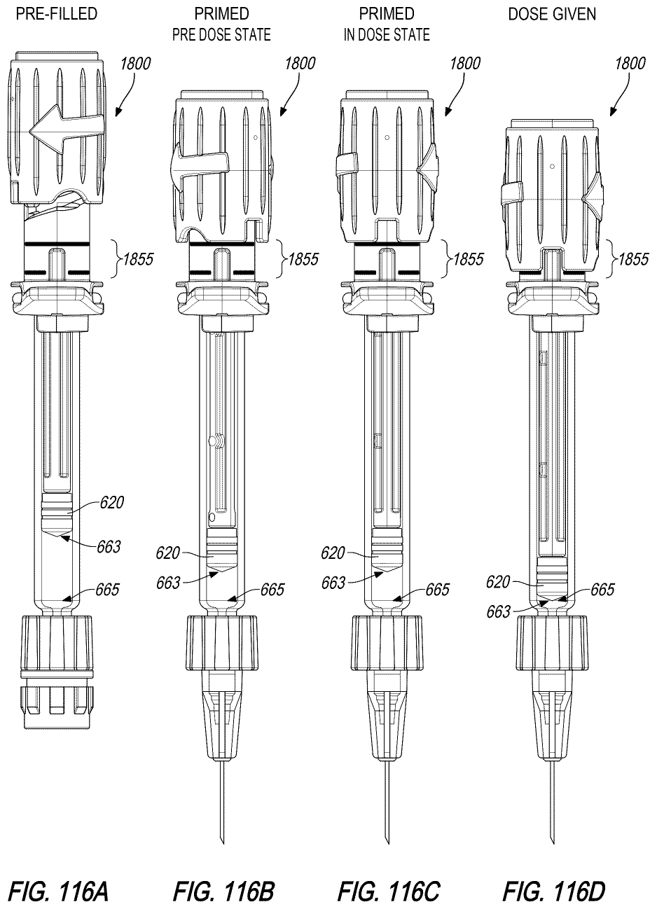

FIGS. 114A, 114B, 114C, and 114D are longitudinal cross-sectional views depicting a system for injecting, such as the system 1800 depicted in FIGS. 104 and 105, in various configurations according to some embodiments. FIGS. 115A, 115B, 115C, and 115D are front views depicting a system for injecting, such as the system 1800 depicted in FIGS. 104 and 105, in various configurations according to some embodiments. FIGS. 116A, 116B, 116C, and 116D are side views depicting a system for injecting, such as the system 1800 depicted in FIGS. 104 and 105, in various configurations according to some embodiments. In FIGS. 114A, 115A, 116A, the system 1800 is in the transport configuration. In FIGS. 114B, 115B, 116B, the system 1800 is in the almost primed/injection ready configuration. In FIGS. 114C, 115C, 116C, the system 1800 is in the primed/injection ready configuration. In FIGS. 114D, 115D, 116D, the system 1800 is in the injected configuration. The plunger rod length is configured such that when contact is made between the distally facing surfaces 1851 of the shroud 1850 and the proximally facing surfaces 1843 of the finger flange 1840, the distal surface 1863 of the stopper 1820 is spaced from the syringe barrel bottom surface 1865. This spacing allows the volume of the delivered dose to be independent of the manufacturing tolerance of the syringe barrel length, allowing enhanced precision and accuracy of the delivered dose volume.

FIGS. 115A-116D show, in detail, external indicia 1855 of a configuration of the system 1800 and its transitions between the transport configuration (see e.g., FIGS. 115A, 116A), the primed/injection ready configuration (see e.g., FIGS. 115C, 116C), and the injected configuration (see e.g., FIGS. 115D, 116D).

As shown in FIG. 115A, when the system 1800 is in the transport configuration, the primed/injection ready configuration indicia 1855-1, the primed/injection ready configuration indicia line 1855-2, the injected configuration indicia 1855-3, and the injected configuration indicia line 1855-4 are all visible from the front view because the shroud 1850 is in a proximal position on the proximally extending member 1842 of the finger flange 1840. In fact, the is an axial space between the distal surface 1851 of the shroud 1850 and the primed/injection ready configuration indicia 1855-1 and the primed/injection ready configuration indicia line 1855-2 because of the proximal position of the shroud 1850.

As shown in FIGS. 115B and 116B, when the system 1800 is in the almost primed configuration, the primed/injection ready configuration indicia 1855-1 and the primed/injection ready configuration indicia line 1855-2 remain visible from the front view. As a result of rotation of the shroud 1850 relative to the finger flange 1840 of the finger flange 1840, the shroud 1850 has advanced distally on the proximally extending member 1842 until the distal surface 1851 of the shroud 1850 is aligned with the primed/injection ready configuration indicia 1855-1 and the corresponding primed/injection ready configuration indicia line 1855-2 (see FIG. 116B). Further, when the system 1800 is in the almost primed configuration, the partial window 1859 is not rotationally aligned with the primed/injection ready configuration indicia 1855-2.

As shown in FIGS. 115C and 116C, when the system 1800 is in the primed/injection ready configuration, the primed/injection ready configuration indicia 1855-1 and the primed/injection ready configuration indicia line 1855-2 remain visible from the front view and aligned with the distal surface 1851 of the shroud 1850 because the shroud 1850 is in same distal position on the proximally extending member 1842 of the finger flange 1840 as when the system 1800 is in the primed/injection ready configuration depicted in FIGS. 115B and 116B. The corresponding primed/injection ready configuration indicia line 1855-2 is also aligned with the distal surface 1851 of the shroud 1850 (see FIG. 116C). In the primed/injection ready configuration depicted in FIGS. 115C and 116C, continued rotation of the shroud 1850 relative to the finger flange 1840 of the finger flange 1840 rotationally aligns the partial window 1859 with the primed/injection ready configuration indicia 1855-2 to indicate that the system 1800 is in the primed/injection ready configuration.

As shown in FIGS. 115D and 116D, when the system 1800 is in the injected configuration, the injected configuration indicia 1855-3, but not the primed/injection ready configuration indicia 1855-1, is visible from the front view because the shroud 1850. As a result of distal movement of the shroud 1850 relative to the finger flange 1840 of the finger flange 1840, the shroud 1850 has advanced further distally on the proximally extending member 1842 until the shroud 1850 covers the primed/injection ready configuration indicia 1855-1 and the distal surface 1851 of the shroud 1850 is aligned with the injected configuration indicia 1855-3 and the corresponding injected configuration indicia line 1855-4 (see FIG. 116D). Further, when the system 1800 is in the injected configuration, the partial window 1859 remains rotationally aligned with the injected configuration indicia 1855-3.

During manufacturing, shipping, and storage prior to use the stopper 1820 may experience additional initial break loose friction also known as stiction with the syringe body 1810. This stiction results in an increased amount of axial force required to break the stopper 1820 free from the syringe body 1810 during priming. This stiction may also be increased by the desire to limit silicone oil lubrication on the inside of the syringe body 1810 for ophthalmological and or intravitreal injections. The priming mechanism described herein provides a mechanical advantage to aide in breaking loose the stopper during priming. In particular, the shroud projection 1856 is configured to engage the helical grooves/threads 1860 of the finger flange 1840 providing a threaded connection between the shroud 1850 and the finger flange 1840. This threaded connection provides a mechanical advantage to the user allowing the stopper 1820 to be freed from the initial stiction with a break loose force applied tangential to the outside diameter of the shroud 1850 by the user to break loose the stopper 1820 from the syringe body 1810. As the plunger member 1830 is advanced distally relative to the finger flange 1840 the system 1800 is positioned in an upward orientation to allow an advancing stopper member (not shown) to eject air from a syringe interior (not shown).

While indicia 1855 are shown in system 1800 depicted in FIGS. 104 and 116D, in other embodiments, injections systems similar to system 1800 may include other system configuration indicia, including but not limited to, those depicted and described herein.

Like many of the injection systems described in co-owned U.S. patent application Ser. No. 14/696,342, Ser. No. 14/543,787, Ser. No. 14/321,706, 62/416,102, 62/431,382, 62/480,276, 62/508,508, 62/542,230, Ser. No. 15/801,239, Ser. No. 15/801,259, Ser. No. 15/801,281, Ser. No. 15/801,304, Ser. No. 16/011,453, 16/683, 126, and 16/683,157, the contents of which were previously fully incorporated herein by reference as though set forth in full, the systems 600, 700, 1300, 1300′, 1600, 1700, 1800 utilizes off-the-shelf syringe bodies 610, 710, 1310, 1610, 1710, 1810, stopper members 620, 720, 1320, 1620, 1720, 1820, and needle connection members. The injection systems 600, 700, 1300, 1300′, 1600, 1700, 1800 may also be used with off-the-shelf needle assemblies including needle hubs and needles.

While various embodiments have been described with specific connectors (e.g., slip and Luer), these embodiments can be used with any known injection system connectors. While various embodiments have been described with staked needles and needle connectors, these embodiments can be used with any known permanently coupled needle or needle connector system.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element-- irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A system for injecting, comprising:
a syringe body having proximal and distal ends, a syringe interior, and a syringe flange at the proximal end thereof;
an injectable fluid disposed in the syringe interior;
a stopper member disposed in the syringe interior;
a plunger member having a distal end configured to be disposed adjacent to the stopper member;
a finger flange removably coupled to the syringe flange; and
a shroud movable relative to the finger flange,
wherein the plunger member is coupled to the shroud.

2. The system of claim 1, wherein the plunger member is rotatably and translationally coupled to the shroud.

3. The system of claim 1, wherein the shroud is rotatable relative to the finger flange.

4. The system of claim 1, wherein the distal end of the plunger member is coupled to the stopper member.

5. The system of claim 1, wherein the finger flange comprises a proximally extending member having an external thread defined thereon.

6. The system of claim 5, wherein the shroud defines an inwardly extending member disposed in the external thread and configured to travel along the external thread.

7. The system of claim 6, wherein the proximally extending member defines a distally extending external longitudinal slot at a distal end of the external thread.

8. The system of claim 7, wherein the proximally extending member defines a bump at the distal end of the external thread,
wherein the bump is configured to cooperate with the inwardly extending member to generate a tactile and/or audible indicator.

9. The system of claim 6, wherein the proximally extending member defines a bump adjacent a proximal end of the external thread,
wherein the bump is configured to cooperate with the inwardly extending member to generate a tactile and/or audible indicator.

10. The system of claim 7, wherein the proximally extending member defines a deflectable detent adjacent a proximal end of the external thread,
wherein the deflectable detent is configured to cooperate with the inwardly extending member to control movement of the inwardly extending member between the external thread and the distally extending external longitudinal slot.

11. The system of claim 10, wherein the deflectable detent is configured to deflect radially

12. The system of claim 10, wherein the deflectable detent is configured to deflect longitudinally/axially

13. The system of claim 10, wherein the deflectable detent is configured to deflect to allow the inwardly extending member to move from the external thread to the distally extending external longitudinal slot.

14. The system of claim 10, wherein the deflectable detent is configured to:
elastically deform with application of a deflecting force to allow the inwardly extending member to move from the external thread to the distally extending external longitudinal slot, and
return to an original position after the deflecting force is removed therefrom.

15. The system of claim 10, wherein the deflectable detent is biased to return to an original/neutral position after a deflecting force is removed therefrom.

16. The system of claim 15, wherein the deflectable detent returning to the original/neutral position prevents the inwardly extending member from moving from the distally extending external longitudinal slot to the external thread.

17. The system of claim 10, wherein the deflectable detent is self-energizing to return to an original/neutral position after a deflecting force is removed therefrom.

18. The system of claim 10, wherein the deflectable detent is configured to cooperate with the inwardly extending member to generate a tactile and/or audible indicator.

19. The system of claim 6, wherein the proximally extending member defines a bump adjacent a proximal end of the external thread,
wherein the bump is configured to cooperate with the inwardly extending member to generate a tactile and/or audible indicator.

20. The system of claim 5, wherein the proximally extending member defines a distally extending internal longitudinal slot.

21. The system of claim 20, wherein the plunger member defines an elongate member outwardly extending from an exterior surface thereof, and
wherein the elongate member of the plunger member is configured to travel along the distally extending internal longitudinal slot of the proximally extending member.

22. The system of claim 5,
wherein the finger flange defines a flange proximally facing surface, and
wherein the finger flange comprises a flange elongate member proximally extending from the flange proximally facing surface,
wherein the flange elongate member is disposed partially along an outer surface of the proximally extending member of the finger flange.

23. The system of claim 22,
wherein the flange elongate member defines a flange elongate member proximally facing surface, and
wherein the shroud defines a shroud distally facing surface configured to interfere with the flange elongate member proximally facing surface to limit distal movement of the shroud to a first longitudinal position on the finger flange when the shroud is in a first rotational state relative to the finger flange.

24. The system of claim 23,
wherein the shroud defines a slot extending proximally from the distally facing surface and configured to receive the flange elongate member to allow distal movement of the shroud distally beyond the first longitudinal position on the finger flange when the shroud is in a second rotational state relative to the finger flange,
wherein the shroud distally facing surface is configured to interfere with the flange proximally facing surface to limit distal movement of the shroud to a second longitudinal position on the finger flange when the shroud is in the second rotational state relative to the finger flange, and
wherein the second longitudinal position is distal of the first longitudinal position.

25. The system of claim 24, wherein the system has:
a transport configuration in which the plunger member is in a proximal most position,
a primed/injection ready configuration in which the plunger member is in an intermediate position, and
an injected configuration in which the plunger member is in a distal most position.

26. The system of claim 25, wherein the slot defined by the shroud is configured to align with the flange elongate member on the finger flange when the system is in the primed/injection ready configuration.

27. The system of claim 25, wherein the finger flange comprises
a first external indicia of a first system transition between the transport configuration and the primed/injection ready configuration, and
a second external indicia of a second system transition between the primed/injection ready configuration and the injected configuration.

28. The system of claim 27,
wherein the shroud does not cover the first external indicia or the second external indicia when the system is in the transport configuration,
wherein the shroud covers the first external indicia and does not cover the second external indicia when the system is in the primed/injection ready configuration, and
wherein the shroud covers the first external indicia and the second external indicia when the system is in the injected configuration.

29. The system of claim 27, wherein the first external indicia has a first color,
wherein the second external indicia has a second color, and
wherein the first color is different from the second color.

30. The system of claim 27, wherein the first external indicia has a first pattern,
wherein the second external indicia has a second pattern, and
wherein the first pattern is different from the second pattern.

31. The system of claim 27, wherein the first external indicia has a first texture,
wherein the second external indicia has a second texture, and
wherein the first texture is different from the second texture.

32. The system of claim 1,
wherein the finger flange defines a flange proximally facing surface,
wherein the shroud defines a shroud distally facing surface configured to interfere with the flange proximally facing surface to limit distal movement of the shroud relative to the finger flange.

33. The system of claim 1, wherein the shroud defines an external indicia of rotation direction.

34. The system of claim 1, wherein the plunger member comprises a pair of radially outwardly extending members adjacent the thumbpad, and
wherein the plunger member defines a pair of grooves adjacent the pair of radially outwardly extending members.

35. The system of claim 34, wherein the shroud defines an opening at a proximal end thereof configured to allow the pair of radially outwardly extending members to pass therethrough to couple to the plunger member to the shroud.

36. The system of claim 1, wherein the plunger member comprises a thumbpad at a proximal end thereof.

37.-71. (canceled)
